SEQUENCE LISTING (42 sequences)

SEQ ID NO: 1 - skipped

SEQ ID NO: 2 - DNA, 14048 bp, synthetic construct
  misc_feature 1..14048: note = AAV Vector Construct
  source 1..14048: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaataacttcgtatagcatacattatacgaagttatgataagagacggtggtggcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccactgcatgacgtctccacaattaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagagccaccatgtacccatacgatgttccagattacgctatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagttgctagcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccagcacactggcggccgttactagcttctgcagcacgaccggttgataatagataacttcgtatagcatacattatacgaagttatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 3 - DNA, 7340 bp, synthetic construct
  misc_feature 1..7340: note = AAV Vector Construct
  source 1..7340: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgtacccatacgatgttccagattacgctgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggag

SEQ ID NO: 4 - DNA, 6095 bp, synthetic construct
  misc_feature 1..6095: note = AAV Vector Construct
  source 1..6095: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccagatctaggaacctagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagcgcgccccgcctagcccgttttagtactctggaaacagaatctactaaaacaaggcaaaatgccgtgtttatctcgtcaacttgttggcgagatttttttgcggccgcccgcggtggagctccagcttttgttccctttagtgagggttaattctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 5 - DNA, 7025 bp, synthetic construct
  misc_feature 1..7025: note = AAV Vector Construct
  source 1..7025: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagtactctggaaacagaatctactaaaacaaggcaaaatgccgtgtttatctcgtcaacttgttggcgagatttttttgcggccgcccgcggtggagctccagcttttgttccctttagtgagggttaattctagagagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctcgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 6 - DNA, 22 bp, synthetic construct
  misc_feature 1..22: note = protospace sequence for CAG Luciferase gRNAs
  source 1..22: mol_type = other DNA
gtcattattgacgtcaatgggc

SEQ ID NO: 7 - DNA, 20 bp, synthetic construct
  misc_feature 1..20: note = protospace sequence for CAG Luciferase gRNAs
  source 1..20: mol_type = other DNA
gtgctcagcaactcggggag

SEQ ID NO: 8 - DNA, 20 bp, synthetic construct
  misc_feature 1..20: note = protospace sequence for CAG Luciferase gRNAs
  source 1..20: mol_type = other DNA
ctcggggaggggggtgcagg

SEQ ID NO: 9 - DNA, 22 bp, synthetic construct
  misc_feature 1..22: note = protospace sequence for CAG Luciferase gRNAs
  source 1..22: mol_type = other DNA
actttccattgacgtcaatggg

SEQ ID NO: 10 - DNA, 22 bp, synthetic construct
  misc_feature 1..22: note = protospace sequence for CAG Luciferase gRNAs
  source 1..22: mol_type = other DNA
cttcgggggggacggggcaggg

SEQ ID NO: 11 - DNA, 21 bp, synthetic construct
  misc_feature 1..21: note = protospace sequence for CAG Luciferase gRNAs
  source 1..21: mol_type = other DNA
cttcgccccgcgcccgctaga

SEQ ID NO: 12 - DNA, 19 bp, synthetic construct
  misc_feature 1..19: note = protospace sequence for CAG Luciferase gRNAs
  source 1..19: mol_type = other DNA
tcggggaggggggtgcagg

SEQ ID NO: 13 - DNA, 19 bp, synthetic construct
  misc_feature 1..19: note = protospace sequence for CAG Luciferase gRNAs
  source 1..19: mol_type = other DNA
tgctcagcaactcggggag

SEQ ID NO: 14 - DNA, 19 bp, synthetic construct
  misc_feature 1..19: note = protospace sequence for CAG Luciferase gRNAs
  source 1..19: mol_type = other DNA
gcggggggtggcggcaggt

SEQ ID NO: 15 - DNA, 19 bp, synthetic construct
  misc_feature 1..19: note = protospace sequence for Mouse Acvr2b gRNAs
  source 1..19: mol_type = other DNA
gctcctctgggacccctga

SEQ ID NO: 16 - DNA, 19 bp, synthetic construct
  misc_feature 1..19: note = protospace sequence for Mouse Acvr2b gRNAs
  source 1..19: mol_type = other DNA
tgctatggagcccacgcta

SEQ ID NO: 17 - DNA, 19 bp, synthetic construct
  misc_feature 1..19: note = protospace sequence for Mouse Acvr2b gRNAs
  source 1..19: mol_type = other DNA
ggcgcgctctccgagctgg

SEQ ID NO: 18 - DNA, 19 bp, synthetic construct
  misc_feature 1..19: note = protospace sequence for Mouse Acvr2b gRNAs
  source 1..19: mol_type = other DNA
agcgcgccccgcctagccc

SEQ ID NO: 19 - DNA, 19 bp, synthetic construct
  misc_feature 1..19: note = protospace sequence for Mouse Acvr2b gRNAs
  source 1..19: mol_type = other DNA
gcctctttgtatccaacat

SEQ ID NO: 20 - DNA, 23 bp, synthetic construct
  misc_feature 1..23: note = protospace sequence for Mouse Acvr2b gRNAs
  source 1..23: mol_type = other DNA
gcacgctcctctgggacccctga

SEQ ID NO: 21 - DNA, 19 bp, synthetic construct
  misc_feature 1..19: note = protospace sequence for Mouse Acvr2b gRNAs
  source 1..19: mol_type = other DNA
gtgggggaggggacctgaa

SEQ ID NO: 22 - DNA, 19 bp, synthetic construct
  misc_feature 1..19: note = protospace sequence for Mouse Acvr2b gRNAs
  source 1..19: mol_type = other DNA
gaggggccatgaacggggg

SEQ ID NO: 23 - DNA, 3600 bp, synthetic construct
  misc_feature 1..3600: note = HA-NLS-dSaCas9-NLS-KRAB
  source 1..3600: mol_type = other DNA
atgtacccatacgatgttccagattacgctgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttaa

SEQ ID NO: 24 - protein, 1368 aa, Streptococcus pyogenes [sequence omitted]

SEQ ID NO: 25 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 26 - protein, 1107 aa, Eubacterium ventriosum
MGYTVGLDIGVASVGVAVLDENDNIVEAVSNIFDEADTSNNKVRRTLREGRRTKRRQKTRIEDFKQLWETSGYIIPHKLHLNIIELRNKGLTELLSLDELYCVLLSMLKHRGISYLEDADDGEKGNAYKKGLAFNEKQLKEKMPCEIQLERMKKYGKYHGEFIIEINDEKEYQSNVFTTKAYKKELEKIFETQRCNGNKINTKFIKKYMEIYERKREYYIGPGNEKSRTDYGIYTTRTDEEGNFIDEKNIFGKLIGKCSVYPEEYRASSASYTAQEFNLLNDLNNLKINNEKLTEFQKKEIVEIIKDASSVNMRKIIKKVIDEDIEQYSGARIDKKGKEIYHTFEIYRKLKKELKTINVDIDSFTREELDKTMDILTLNTERESIVKAFDEQKFVYEENLIKKLIEFRKNNQRLFSGWHSFSYKAMLQLIPVMYKEPKEQMQLLTEMNVFKSKKEKYVNYKYIPENEVVKEIYNPVVVKSIRTTVKILNALIKKYGYPESVVIEMPRDKNSDDEKEKIDMNQKKNQEEYEKILNKIYDEKGIEITNKDYKKQKKLVLKLKLWNEQEGLCLYSGKKIAIEDLLNHPEFFEIDHIIPKSISLDDSRSNKVLVYKTENSIKENDTPYHYLTRINGKWGFDEYKANVLELRRRGKIDDKKVNNLLCMEDITKIDVVKGFINRNLNDTRYASRVVLNEMQSFFESRKYCNTKVKVIRGSLTYQMRQDLHLKKNREESYSHHAVDAMLIAFSQKGYEAYRKIQKDCYDFETGEILDKEKWNKYIDDDEFDDILYKERMNEIRKKIIEAEEKVKYNYKIDKKCNRGLCNQTIYGTREKDGKIHKISSYNIYDDKECNSLKKMINSGKGSDLLMYNNDPKTYRDMLKILETYSSEKNPFVAYNKETGDYFRKYSKNHNGPKVEKVKYYSGQINSCIDISHKYGHAKNSKKVVLVSLNPYRTDVYYDNDTGKYYLVGVKYNHIKCVGNKYVIDSETYNELLRKEGVLNSDENLEDLNSKNITYKFSLYKNDIIQYEKGGEYYTERFLSRIKEQKNLIETKPINKPNFQRKNKKGEWENTRNQIALAKTKYVGKLVTDVLGNCYIVNMEKFSLVVDK

SEQ ID NO: 27 - protein, 1168 aa, Azospirillum sp.
MARPAFRAPRREHVNGWTPDPHRISKPFFILVSWHLLSRVVIDSSSGCFPGTSRDHTDKFAEWECAVQPYRLSFDLGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDHILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 28 - protein, 1050 aa, Gluconacetobacter diazotrophicus
MGENMIDESLTFGIDLGIGSCGWAVLRRPSAFGRKGVIEGMGSWCFDVPETSKERTPTNQIRRSNRLLRRVIRRRRNRMAAIRRLLHAAGLLPSTDSDALKRPGHDPWELRARGLDKPLKPVEFAVVLGHIAKRRGFKSAAKRKATNISSDDKKMLTALEATRERLGRYRTVGEMFARDPDFASRRRNREGKYDRTTARDDLEHEVHALFAAQRRLGQGFASPELEEAFTASAFHQRPMQDSERLVGFCPFERTEKRAAKLTPSFERFRLLARLLNLRITTPDGERPLTVDEIALVTRDLGKTAKLSIKRVRTLIGLEDNQRFTTIRPEDEDRDIVARTGGAMTGTATLRKALGEALWTDMQERPEQLDAIVQVLSFFEANETITEKLREIGLTLAVLDVLLTALDAGVFAKFKGAAHISTKAARNLLPHLEQGRRYDEACTMAGYDHAASRLSHHGQIVAKTQFNALVTEIGESIANPIARKALIEGLKQIWAMRNHWGLPGSIHVELARDVGNSIEKRREIEKHIEKNTALRARERREVHDLLDLEDVNGDTLLRYRLWKEQGGKCLYTGKAIHIRQIAATDNSVQVDHILPWSRFGDDSFNNKTLCLASANQQKKRSTPYEWLSGQTGDAWNAFVQRIETNKELRGFKKRNYLLKNAKEAEEKFRSRNLNDTRYAARLFAEAVKLLYAFGERQEKGGNRRVFTRPGALTAALRQAWGVESLKKQDGKRINDDRHHALDALTVAAVDEAEIQRLTKSFHEWEQQGLGRPLRRVEPPWESFRADVEATYPEVFVARPERRRARGEGHAATIRQVKERECTPIVFERKAVSSLKEADLERIKDGERNEAIVEAIRSWIATGRPADAPPRSPRGDIITKIRLATTIKAAVPVRGGTAGRGEMVRADVFSKPNRRGKDEWYLVPVYPHQIMNRKAWPKPPMRSIVANKDEDEWTEVGPEHQFRFSLYPRSNIEIIRPSGEVIEGYFVGLHRNTGALTISAHNDPKSIHSGIGTKTLLAISKYQVDRFGRKSPVRKEVRTWHGEACISPTPPG

SEQ ID NO: 29 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 30 - protein, 1140 aa, Roseburia intestinalis
MRENGSDERRRNMDEKMDYRIGLDIGIASVGWAVLQNNSDDEPVRIVDLGVRIFDTAEIPKTGESLAGPRRAARTTRRRLRRRKHRLDRIKWLFENQGLINIDDFLKRYNMAGLPDVYQLRYEALDRKLTDEELAQVLLHIAKHRGFRSTRKAETAAKENGAVLKATDENQKRMQEKGYRTVGEMIYLDEAFRTGCSWSEKGYILTPRNKAENYQHTMLRAMLVEEVKEIFSSQRRLGNEKATEELEEKYLEIMTSQRSFDLGPGMQPDGKPSPYAMEGFSDRVGKCTFLGDQGELRGAKGTYTAEYFVALQKINHTKLVNQDGETRNFTEEERRALTLLLFTQKEVKYAAVRKKLGLPEDILFYNLNYKKAATKEEQQKENQNTEKAKFIGMPYYHDYKKCLEERVKYLTENEVRDLFDEIGMILTCYKNDDSRTERLAKLGLVPIEMEGLLAYTPTKFQHLSMKAMRNIIPFLEKGMTYDKACEEAGYDFKADSKGTKQKLLTGENVNQTINEITNPVVKRSVSQTVKVINAIIRTYGSPQAINIELAREMSKTFEERRKIKGDMEKRQKNNEDVKKQIQELGKLSPTGQDILKYRLWQEQQGICMYSGKTIPLEELFKPGYDIDHILPYSITFDDSFRNKVLVTSQENRQKGNRTPYEYMGNDEQRWNEFETRVKTTIRDYKKQQKLLKKHFSEEERSEFKERNLTDTKYITTVIYNMIRQNLEMAPLNRPEKKKQVRAVNGAITAYLRKRWGLPQKNRETDTHHAMDAVVIACCTDGMIQKISRYTKVRERCYSKGTEFVDAETGEIFRPEDYSRAEWDEIFGVHIPKPWETFRAELDVRMGDDPKGFLDTHSDVALELDYPEYIYENLRPIFVSRMPNHKVTGAAHADTIRSPRHFKDEGIVLTKTALTDLKLDKDGEIDGYYNPQSDLLLYEALKKQLLLYGNDAKKAFAQDFHKPKADGTEGPVVRKVKIQKKQTMGVFVDSGNGIAENGGMVRIDVFRVNGKYYFVPVYTADVVKKVLPNRASTAHKPYGEWKVMEDKDFLFSLYSRDLIHIKSKKDIPIKMVNGGMEGIKETYAYYIGADISAANIQGIAHDSRYKFRGLGIQSLDVLEKCQIDVLGHVSVVRSEKRMGFS

SEQ ID NO: 31 - protein, 1037 aa, Parvibaculum lavamentivorans
MERIFGFDIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEHIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 32 - protein, 1132 aa, Nitratifractor salsuginis
MKKILGVDLGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEHIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 33 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 34 - protein, 103 aa, synthetic construct
  REGION 1..103: note = exemplary KRAB
DAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 35 - protein, 1052 aa, synthetic construct
  REGION 1..1052: note = exemplary KRAB
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 36 - protein, 1053 aa, synthetic construct
  REGION 1..1053: note = dCas9 sequence
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 37 - protein, 16 aa, synthetic construct
  REGION 1..16: note = exemplary nuclear localization sequence
APKKKRKVGIHGVPAA

SEQ ID NO: 38 - protein, 16 aa, synthetic construct
  REGION 1..16: note = exemplary nuclear localization sequence
KRPAATKKAGQAKKKK

SEQ ID NO: 39 - protein, 1199 aa, synthetic construct
  REGION 1..1199: note = exemplary HA-NLS-dSaCas9-NLS-KRAB sequence
MYPYDVPDYAAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 40 - protein, 1198 aa, synthetic construct
  REGION 1..1198: note = exemplary HA-NLS-dSaCas9-NLS-KRAB sequence
YPYDVPDYAAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 41 - protein, 1189 aa, synthetic construct
  REGION 1..1189: note = exemplary NLS-dSaCas9-NLS-KRAB
APKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 42 - DNA, 22 bp, synthetic construct
  misc_feature 1..22: note = protospacer sequence
  source 1..22: mol_type = other DNA
gagggaagggatacaggctgga

(19) United States
(12) Patent Application Publication
(10) Pub. No.: US 2026/0055386 A1
(43) Pub. Date: Feb. 26, 2026

(54) CRISPR/CAS9-BASED REPRESSORS FOR SILENCING GENE TARGETS IN VIVO AND METHODS OF USE

(71) Applicant: Duke University, Durham, NC (US)

(72) Inventors: Charles A. Gersbach, Chapel Hill, NC (US); Pratiksha I. Thakore, Durham, NC (US)

(21) Appl. No.: 19/310,493

(22) Filed: Aug. 26, 2025

Related U.S. Application Data
(63) Continuation of application No. 18/405,995, filed on Jan. 5, 2024, now Pat. No. 12,428,631.
Application No. 18/405,995 is a continuation of application No. 16/093,272, now abandoned.
(60) Provisional application No. 62/321,947, filed on Apr. 13, 2016.
(60) Provisional application No. 62/369,248, filed on Aug. 1, 2016.

(51) Int. Cl. C12N 9/22; A61K 9/00; A61K 48/00; A61P 9/00; C07K 14/47; C12N 7/00; C12N 15/11; C12N 15/113; C12N 15/52; C12N 15/63; C12N 15/86

(52) U.S. Cl. CPC C12N 9/22; A61K 9/0019; A61K 48/0058; A61P 9/00; C07K 14/4703; C12N 7/00; C12N 15/11; C12N 15/111; C12N 15/113; C12N 15/52; C12N 15/63; C12N 15/86; A61K 48/00; C07K 2319/09; C07K 2319/71; C07K 2319/80; C12N 2310/20; C12N 2320/32; C12N 2750/14143; C12N 2800/80

(57) ABSTRACT

The present disclosure provides Crispr/cas9-based repressors for silencing gene targets in vivo and methods of use.

### CROSS REFERENCE TO RELATED APPLICATIONS

This patent application is continuation of U.S. patent application Ser. No. 18/405,995, filed Jan. 5, 2024, which is a continuation of U.S. patent application Ser. No. 16/093,272, filed Oct. 12, 2018, which is the national stage filing under 35 U.S.C. § 371 of International Patent Application No. PCT/US2017/027490, filed Apr. 13, 2017, which claims the benefit of U.S. Provisional Application No. 62/321,947, filed Apr. 13, 2016, and U.S. Provisional Application No. 62/369,248, filed Aug. 1, 2016, the entire contents of each of which are hereby incorporated by reference.

### STATEMENT OF GOVERNMENT INTEREST

This invention was made with Government support under Federal Grant Nos. 1 R01 DA036865 and 1 DP2 OD008586 awarded by the National Institutes of Health. The Government has certain rights to this invention.

### SEQUENCE LISTING

The application includes a Sequence Listing which has been submitted electronically in XML format and is hereby incorporated by reference in its entirety. Said XML Sequence Listing, created on Aug. 25, 2025, is named 028193-9264-US06 Sequence Listing.xml, and is 1,144,410 bytes in size.

### BACKGROUND

Engineered DNA-binding proteins that can be customized to target any gene in mammalian cells have enabled rapid advances in biomedical research and are a promising platform for gene therapies. The RNA-guided CRISPR-Cas9 system has emerged as a promising platform for programmable targeted gene regulation. Fusion of catalytically inactive, “dead” Cas9 (dCas9) to the Kruppel-associated box (KRAB) domain generates a synthetic repressor capable of highly specific and potent silencing of target genes in cell culture experiments. However, a technology to deliver CRISPR/Cas9-based gene repressors in vivo has not been developed. Adeno-associated virus (AAV) vectors have been proposed for gene delivery of CRISPR-Cas9 components for in vivo studies and therapeutic applications. AAV vectors provide stable gene expression with low risk of mutagenic integration events. AAV vectors can be engineered to target tissues of interest in vivo, and are already in use in humans in clinical trials. However, gene delivery of S. pyogenes dCas9-KRAB in vivo is challenging because the size of the S. pyogenes dCas9 and KRAB domain fusion exceeds the packaging limits of standard AAV vectors.

### SUMMARY

In an aspect, the disclosure features a method of modulating expression of a gene, in vivo, in a subject comprising administering to, or providing in, the subject:
(a)(i) a fusion molecule comprising a sequence comprising a dCas9 molecule fused to a modulator of gene expression; or (ii) a nucleic acid that encodes a fusion molecule comprising a sequence comprising a dCas9 molecule fused to a modulator of gene expression; and
(b)(i) a gRNA which targets the fusion molecule to the gene; or (ii) a nucleic acid that encodes a gRNA which targets the fusion molecule to the gene,
   in an amount sufficient to modulate expression of the gene.

In an embodiment, the method comprises administering to, or provided in, the subject any of: (a)(ii) and (b)(ii), (a)(i) and (b)(i), (a)(i) and (b)(ii), or (a)(ii) and (b)(i).

In an embodiment, the method comprises administering to, or provided in, the subject:
(a)(ii) a nucleic acid that encodes a fusion molecule comprising a sequence comprising a dCas9 molecule fused to a modulator of gene expression; and
(b)(ii) a nucleic acid that encodes a gRNA which targets the fusion molecule to the gene.

In an embodiment, the nucleic acid of (a)(ii) comprises DNA. In an embodiment, the nucleic acid of (b)(ii) comprises DNA. In an embodiment, the nucleic acid of (a)(ii) comprises RNA. In an embodiment, the nucleic acid of (b)(ii) comprises RNA.

In an embodiment, the method comprises one or both of (a) and (b) are packaged in a viral vector. In an embodiment, (a) is packaged in a viral vector. In an embodiment, (b) is packaged in a viral vector. In an embodiment, (a) and (b) are packaged in the same viral vector.

In an embodiment, the viral vector comprises an AAV vector. In an embodiment, the viral vector comprises a lentiviral vector.

In an embodiment, (a) is packaged in a first viral vector and (b) is packaged in a second viral vector. In an embodiment, the first viral vector comprises an AAV vector and the second viral vector comprises an AAV vector.

In an embodiment, the dCas9 molecule comprises a gRNA binding domain of a Cas9 molecule. In an embodiment, the dCas9 molecule comprises one, two or all of: a Rec1 domain, a bridge helix domain, or a PAM interacting domain, of a Cas9 molecule. In an embodiment, the dCas9 molecule is a mutant of a wild-type Cas9 molecule, e.g., in which the Cas9 nuclease activity is inactivated. In an embodiment, the dCas9 molecule comprises a mutation that inactivates a Cas9 nuclease activity, e.g., a mutation in a DNA-cleavage domain of a Cas9 molecule. In an embodiment, the dCas9 molecule comprises a mutation that inactivates a Cas9 nuclease activity, e.g., a mutation in a RuvC domain and/or a mutation in a HNH domain.

In an embodiment, the dCas9 molecule comprises a Staphylococcus aureus dCas9 molecule, a Streptococcus pyogenes dCas9 molecule, a Campylobacter jejuni dCas9 molecule, a Corynebacterium diphtheria dCas9 molecule, a Eubacterium ventriosum dCas9 molecule, a Streptococcus pasteurianuis dCas9 molecule, a Lactobacillus farciminis dCas9 molecule, a Sphaerochaeta globus dCas9 molecule, an Azospirillum (e.g., strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (e.g., strain DSM 16511) dCas9 molecule, a Campylobacter lari (e.g., strain CF89-12) dCas9 molecule, or a Streptococcus thermophilus (e.g., strain LMD-9) dCas9 molecule.

In an embodiment, the dCas9 molecule comprises an S. aureus dCas9 molecule, e.g., comprising an S. aureus dCas9 sequence described herein.

In an embodiment, the S. aureus dCas9 molecule comprises a mutation at an amino acid position, corresponding to position 10, 580, or both (e.g., D10A, N580A, or both), relative to a wild-type S. aureus dCas9 molecule, numbered according to SEQ ID NO: 25.

In an embodiment, the S. aureus dCas9 molecule comprises the amino acid sequence of SEQ ID NO: 35 or 36, a sequence substantially identical (e.g., at least 80%, 85%, 90%, 92%, 95%, 97%, 98%, 99% or higher identical) to SEQ ID NO: 35 or 36, or a sequence having one, two, three, four, five or more changes, e.g., amino acid substitutions, insertions, or deletions, relative to SEQ ID NO: 35 or 36, or any fragment thereof.

In an embodiment, the dCas9 molecule comprises an S. pyogenes dCas9 molecule, e.g., comprising an S. pyogenes dCas9 sequence described herein.

In an embodiment, the S. pyogenes dCas9 molecule comprises a mutation at an amino acid position, corresponding to position 10, 840, or both (e.g., D10A, H840A, or both), relative to a wild-type S. pyogenes dCas9 molecule, numbered according to SEQ ID NO: 24.

In an embodiment, the dCas9 molecule is less than 1400, 1300, 1200, 1100, 1000, 900, 800, 700, 600, or 500 amino acids in length. In an embodiment, the dCas9 molecule is 500-1300, 600-1200, 700-1100, 800-1000, 500-1200, 500-1000, 500-800, 500-600, 1000-1200, 800-1200, or 600-1200 amino acids in length.

In an embodiment, the dCas9 molecule has a size that is less than 90%, 80%, 70%, 60%, 50%, 40%, or 30% of the size of a wild-type Cas9 molecule, e.g., a wild-type S. pyogenes Cas9 molecule or a wild-type S. aureus dCas9 molecule.

In an embodiment, the modulator of gene expression comprises a modulator of gene expression described herein.

In an embodiment, the modulator of gene expression comprises a repressor of gene expression, e.g., a Krüppel associated box (KRAB) molecule, an mSin3 interaction domain (SID) molecule, four concatenated mSin3 interaction domains (SID4X), MAX-interacting protein 1 (MXI1), or any fragment thereof.

In an embodiment, the modulator of gene expression comprises a Krüppel associated box (KRAB) molecule comprising the sequence of SEQ ID NO: 34, a sequence substantially identical (e.g., at least 80%, 85%, 90%, 92%, 95%, 97%, 98%, 99% or higher identical) to SEQ ID NO: 34, or a sequence having one, two, three, four, five or more changes, e.g., amino acid substitutions, insertions, or deletions, relative to SEQ ID NO: 34, or any fragment thereof.

In an embodiment, the modulator of gene expression comprises an activator of gene expression, e.g., a VP16 transcription activation domain, a VP64 transcriptional activation domain, a p65 activation domain, an Epstein-Barr virus R transactivator Rta molecule, a VP64-p65-Rta fusion (VPR), Ldb1 self-association domain, or any fragment thereof.

In an embodiment, the modulator of gene expression comprises a modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., Lys-specific histone demethylase 1 (LSD1)), a DNA methyltransferase (e.g., DNMT3a or DNMT3a-DNMT3L), a DNA demethylase (e.g., TET1 catalytic domain or TDG), or fragment thereof.

In an embodiment, the modulator of gene expression is fused to the C-terminus, N-terminus, or both, of the dCas9 molecule.

In an embodiment, the modulator of gene expression is fused to the dCas9 molecule directly. In an embodiment, the modulator of gene expression is fused to the dCas9 molecule indirectly, e.g., via a non-modulator or a linker, or a second modulator.

In an embodiment, a plurality of modulators of gene expression, e.g., two or more identical, substantially identical, or different modulators, are fused to the dCas9 molecule.

In an embodiment, the fusion molecule further comprises a nuclear localization sequence.

In an embodiment, one or more nuclear localization sequences are fused to the C-terminus, N-terminus, or both, of the dCas9 molecule, e.g., directly or indirectly, e.g., via a linker.

In an embodiment, the one or more nuclear localization sequences comprise the amino acid sequence of SEQ ID NO: 37 or 38, a sequence substantially identical (e.g., at least 80%, 85%, 90%, 92%, 95%, 97%, 98%, 99% or higher identical) to SEQ ID NO: 37 or 38, or a sequence having one, two, three, four, five or more changes, e.g., amino acid substitutions, insertions, or deletions, relative to SEQ ID NO: 37 or 38, or any fragment thereof.

In an embodiment, the fusion molecule comprises the amino acid sequence of SEQ ID NO: 39, 40, or 41, a sequence substantially identical (e.g., at least 80%, 85%, 90%, 92%, 95%, 97%, 98%, 99% or higher identical) to SEQ ID NO: 39, 40, or 41, or a sequence having one, two, three, four, five or more changes, e.g., amino acid substitutions, insertions, or deletions, relative to SEQ ID NO: 39, 40, or 41, or any fragment thereof.

In an embodiment, the nucleic acid that encodes the fusion molecule comprises the sequence of SEQ ID NO: 23, a sequence substantially identical (e.g., at least 80%, 85%, 90%, 92%, 95%, 97%, 98%, 99% or higher identical) to SEQ ID NO: 23, or a sequence having one, two, three, four, five or more changes, e.g., substitutions, insertions, or deletions, relative to SEQ ID NO: 23, or any fragment thereof.

In an embodiment, the gRNA comprises a unimolecular gRNA. In an embodiment, the gRNA comprises a bimolecular gRNA.

In an embodiment, the gRNA comprises a gRNA sequence described herein.

In an embodiment, gene expression is modulated in a cell, tissue, or organ described herein, e.g., Table 2 or 3. In an embodiment, gene expression is modulated in the liver.

In an embodiment, the modulation is sufficient to alter a function of the gene, or a symptom of a disorder associated with the gene, as described herein, e.g., in Table 2 or 3.

In an embodiment, the modulation comprises modulation of transcription. In an embodiment, the modulation comprises down-regulation of transcription. In an embodiment, the modulation comprises up-regulation of transcription.

In an embodiment, the modulation comprises modulating the temporal pattern of expression of the gene. In an embodiment, the modulation comprises modulating the spatial pattern of expression of the gene.

In an embodiment, the modulation comprises modulating a post-transcriptional or co-transcriptional modification, e.g., splicing, 5′ capping, 3′ cleavage, 3′ polyadenylation, or RNA export.

In an embodiment, the modulation comprises modulating the expression of an isoform, e.g., an increase or decrease in the expression of an isoform, the increase or decrease in the expression of a first isoform over a second isoform.

In an embodiment, the modulation comprises modulating chromatin structure, e.g., increasing or decreasing methylation, acetylation, phosphorylation, or ubiquitination, e.g., at a preselected site, or altering the spatial pattern, cell specificity, or temporal occurrence of methylation, acetylation, phosphorylation, or ubiquitination.

In an embodiment, the modulation comprises modulating a post-translational modification (e.g., indirectly), e.g., glycosylation, lipidation, acetylation, phosphorylation, amidation, hydroxylation, methylation, ubiquitination, sulfation, nitrosylation, or proteolysis.

In an embodiment, the modulation does not comprise cleaving the subject's DNA.

In an embodiment, the modulation comprises an inducible modulation.

In an embodiment, the gene is selected from Table 2, optionally wherein the method down-regulates the expression of the gene.

In an embodiment, the gene is selected from Table 3, optionally wherein the method up-regulates the expression of the gene.

In an embodiment, the gene comprises PCSK9.

In an embodiment, the dCas9 molecule does not cleave the genome of the subject.

In another aspect, the disclosure features a method of modulating expression of a gene, in vivo, in a subject comprising administering to, or providing in, the subject:
(a)(ii) a nucleic acid that encodes a fusion molecule comprising a sequence comprising an S. aureus dCas9 molecule fused to a KRAB molecule; and
(b)(ii) a nucleic acid that encodes a gRNA which targets the fusion molecule to the gene, and
   wherein one or both of (a)(i) and (b)(ii) are packaged in an AAV vector.

In an embodiment, the fusion molecule comprises a fusion molecule described herein.

In an embodiment, the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.

In an embodiment, the gRNA comprises a gRNA sequence described herein.

In an embodiment, the gene is selected from Table 2 or 3. In an embodiment, the gene comprises PCSK9.

In an embodiment, (a)(ii) and (b)(ii) are packaged in different AAV vectors. In an embodiment, (a)(ii) and (b)(ii) are packaged in the same AAV vector.

In another aspect, the disclosure features a pharmaceutical composition, or unit dosage form, comprising, in an amount sufficient for modulating a gene in a human subject, or in an amount sufficient for a therapeutic effect in a human subject,
(a)(ii) a nucleic acid that encodes a fusion molecule comprising a sequence comprising a dCas9 molecule, e.g., an S. aureus dCas9 molecule, fused to a modulator of gene expression; and/or
(b)(ii) a nucleic acid that encodes a gRNA which targets the fusion molecule to the gene,
wherein one or both of (a)(ii) and (b)(ii) are packaged in a viral vector.

In an embodiment, the fusion molecule comprises a fusion molecule described herein.

In an embodiment, the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.

In an embodiment, the gRNA comprises a gRNA sequence described herein.

In an embodiment, the gene is selected from Table 2 or 3. In an embodiment, the gene comprises PCSK9.

In an embodiment, one or both of (a)(ii) and (b)(ii) are packaged in an AAV vector.

In an embodiment, (a)(ii) and (b)(ii) are packaged in the same viral vector, e.g., an AAV vector. In an embodiment, (a)(ii) and (b)(ii) are packaged in different viral vectors, e.g., AAV vectors.

In an embodiment, the viral vector (e.g., AAV vector) comprising (a)(ii), and the viral vector (e.g., AAV vector) comprising (b)(ii), are provided in separate containers.

In an embodiment, the viral vector (e.g., AAV vector) comprising (a)(ii) and the viral vector (e.g., AAV vector) comprising (b)(ii), are provided in the same container.

In an embodiment, the pharmaceutical composition, or unit dosage form, is formulated for administration, e.g., oral, parenteral, sublingual, transdermal, rectal, transmucosal, topical, intrapleural, intravenous, intraarterial, intraperitoneal, subcutaneous, intramuscular, intranasal intrathecal, or intraarticular administration, or administration via inhalation or via buccal administration, or any combination thereof, to the subject.

In an embodiment, the pharmaceutical composition, or unit dosage form, is formulated for intravenous administration to the subject.

In an embodiment, the pharmaceutical composition, or unit dosage form, is disposed in a device suitable for administration, e.g., oral, parenteral, sublingual, transdermal, rectal, transmucosal, topical, intrapleural, intravenous, intraarterial, intraperitoneal, subcutaneous, intramuscular, intranasal intrathecal, or intraarticular administration, or administration via inhalation or via buccal administration, or any combination thereof, to the subject.

In an embodiment, the pharmaceutical composition, or unit dosage form, is disposed in a device suitable for intravenous administration to the subject.

In an embodiment, the pharmaceutical composition, or unit dosage form, is disposed in a volume of at least 1, 2, 5, 10, 15, 20, 25, 30, 40, 50, 60, 70, 80, 90, 100, 150, 200, 250, 300, 400, or 500 ml.

In an embodiment, the nucleic acid of (a)(ii) comprises DNA. In an embodiment, the nucleic acid of (b)(ii) comprises DNA. In an embodiment, the nucleic acid of (a)(ii) comprises RNA. In an embodiment, the nucleic acid of (b)(ii) comprises RNA.

In an embodiment, the dCas9 molecule comprises a gRNA binding domain of a Cas9 molecule.

In an embodiment, the dCas9 molecule comprises one, two or all of: a Rec1 domain, a bridge helix domain, or a PAM interacting domain, of a Cas9 molecule. In an embodiment, the dCas9 molecule is a mutant of a wild-type Cas9 molecule, e.g., in which the Cas9 nuclease activity is inactivated. In an embodiment, the dCas9 molecule comprises a mutation that inactivates a Cas9 nuclease activity, e.g., a mutation in a DNA-cleavage domain of a Cas9 molecule. In an embodiment, the dCas9 molecule comprises a mutation that inactivates a Cas9 nuclease activity, e.g., a mutation in a RuvC domain and/or a mutation in a HNH domain.

In an embodiment, the dCas9 molecule comprises a Staphylococcus aureus dCas9 molecule, a Streptococcus pyogenes dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (e.g., strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (e.g., strain DSM 16511) dCas9 molecule, a Campylobacter lari (e.g., strain CF89-12) dCas9 molecule, or a Streptococcus thermophilus (e.g., strain LMD-9) dCas9 molecule.

In an embodiment, the dCas9 molecule comprises an S. aureus dCas9 molecule, e.g., comprising an S. aureus dCas9 sequence described herein. In an embodiment, the S. aureus dCas9 molecule comprises a mutation at an amino acid position, corresponding to position 10, 580, or both (e.g., D10A, N580A, or both), relative to a wild-type S. aureus dCas9 molecule, numbered according to SEQ ID NO: 25.

In an embodiment, the S. aureus dCas9 molecule comprises the amino acid sequence of SEQ ID NO: 35 or 36, a sequence substantially identical (e.g., at least 80%, 85%, 90%, 92%, 95%, 97%, 98%, 99% or higher identical) to SEQ ID NO: 35 or 36, or a sequence having one, two, three, four, five or more changes, e.g., amino acid substitutions, insertions, or deletions, relative to SEQ ID NO: 35 or 36, or any fragment thereof.

In an embodiment, the dCas9 molecule comprises an S. pyogenes dCas9 molecule, e.g., comprising an S. pyogenes dCas9 sequence described herein. In an embodiment, the S. pyogenes dCas9 molecule comprises a mutation at an amino acid position, corresponding to position 10, 840, or both (e.g., D10A, H840A, or both), relative to a wild-type S. pyogenes dCas9 molecule, numbered according to SEQ ID NO: 24.

In an embodiment, the dCas9 molecule is less than 1400, 1300, 1200, 1100, 1000, 900, 800, 700, 600, or 500 amino acids in length.

In an embodiment, the dCas9 molecule is 500-1300, 600-1200, 700-1100, 800-1000, 500-1200, 500-1000, 500-800, 500-600, 1000-1200, 800-1200, or 600-1200 amino acids in length.

In an embodiment, the dCas9 molecule has a size that is less than 90%, 80%, 70%, 60%, 50%, 40%, or 30% of the size of a wild-type Cas9 molecule, e.g., a wild-type S. pyogenes Cas9 molecule or a wild-type S. aureus dCas9 molecule.

In an embodiment, modulator of gene expression comprises a modulator of gene expression described herein.

In an embodiment, modulator of gene expression comprises a KRAB molecule, e.g., comprising the sequence of SEQ ID NO: 34, a sequence substantially identical (e.g., at least 80%, 85%, 90%, 92%, 95%, 97%, 98%, 99% or higher identical) to SEQ ID NO: 34, or a sequence having one, two, three, four, five or more changes, e.g., amino acid substitutions, insertions, or deletions, relative to SEQ ID NO: 34, or any fragment thereof.

In an embodiment, the gRNA comprises a unimolecular gRNA. In an embodiment, the gRNA comprises a bimolecular gRNA. In an embodiment, the gRNA comprises a gRNA sequence described herein.

In an embodiment, gene expression is modulated in a cell, tissue, or organ described herein, e.g., Table 2 or 3. In an embodiment, gene expression is modulated in the liver.

In an embodiment, the modulation is sufficient to alter a function of the gene, or a symptom of a disorder associated with the gene, as described herein, e.g., in Table 2 or 3.

In an embodiment, the modulation comprises modulation of transcription. In an embodiment, the modulation comprises down-regulation of transcription. In an embodiment, the modulation comprises up-regulation of transcription.

In an embodiment, the modulation comprises modulating the temporal pattern of expression of the gene. In an embodiment, the modulation comprises modulating the spatial pattern of expression of the gene.

In an embodiment, the modulation comprises modulating a post-transcriptional or co-transcriptional modification, e.g., splicing, 5′ capping, 3′ cleavage, 3′ polyadenylation, or RNA export.

In an embodiment, the modulation comprises modulating the expression of an isoform, e.g., an increase or decrease in the expression of an isoform, the increase or decrease in the expression of a first isoform over a second isoform.

In an embodiment, the modulation comprises modulating chromatin structure, e.g., increasing or decreasing methylation, acetylation, phosphorylation, or ubiquitination, e.g., at a preselected site, or altering the spatial pattern, cell specificity, or temporal occurrence of methylation, acetylation, phosphorylation, or ubiquitination.

In an embodiment, the modulation comprises modulating a post-translational modification (e.g., indirectly), e.g., glycosylation, lipidation, acetylation, phosphorylation, amidation, hydroxylation, methylation, ubiquitination, sulfation, nitrosylation, or proteolysis.

In an embodiment, the gene is selected from Table 2, optionally wherein the method down-regulates the expression of the gene. In an embodiment, the gene is selected from Table 3, optionally wherein the method up-regulates the expression of the gene. In an embodiment, the gene comprises PCSK9.

In an embodiment, the dCas9 does not cleave the genome of the subject.

In another aspect, the disclosure features a pharmaceutical composition, or unit dosage form, comprising, in an amount sufficient for modulating a gene in a human subject, or in an amount sufficient for a therapeutic effect in a human subject,
(a)(ii) a nucleic acid that encodes a fusion molecule comprising a sequence comprising an S. aureus dCas9 molecule fused to a KRAB molecule; and/or
(b)(ii) a nucleic acid that encodes a gRNA which targets the fusion molecule to the gene,
wherein one or both of (a)(ii) and (b)(ii) are packaged in a viral vector.

In an embodiment, the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.

In an embodiment, the gRNA comprises a gRNA sequence described herein.

In an embodiment, the gene is selected from Table 2 or 3. In an embodiment, the gene comprises PCSK9.

In an embodiment, one or both of (a)(ii) and (b)(ii) are packaged in an AAV vector.

In an embodiment, (a)(ii) and (b)(ii) are packaged in different AAV vectors. In an embodiment, (a)(ii) and (b)(ii) are packaged in the same AAV vector.

In another aspect, the disclosure features a viral vector comprising:
(a)(ii) a nucleic acid that encodes a fusion molecule comprising a sequence comprising a dCas9 molecule fused to a modulator of gene expression; and/or
(b)(ii) a nucleic acid that encodes a gRNA which targets the fusion molecule to a gene.

In an embodiment, the viral vector is an AAV vector.

In an embodiment, the fusion molecule comprises a fusion molecule described herein.

In an embodiment, the dCas9 molecule comprises a dCas9 molecule described herein, e.g., an S. aureus dCas9 molecule.

In an embodiment, the modulator of gene expression comprises a modulator described herein.

In an embodiment, the gene is a gene described herein.

In an embodiment, the viral vector comprises:
(a)(ii) a nucleic acid that encodes a fusion molecule comprising a sequence comprising an S. aureus dCas9 molecule fused to a KRAB molecule; and
(b)(ii) a nucleic acid that encodes a gRNA which targets the fusion molecule to PCSK9,
wherein one or both of (a)(ii) and (b)(ii) are packaged in an AAV vector.

In an embodiment, the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.

In an embodiment, the gRNA comprises a gRNA sequence described herein.

In an embodiment, the gene is selected from Table 2 or 3. In an embodiment, the gene comprises PCSK9.

In an embodiment, the disclosure features a method of treating a disorder, comprising administering to a subject:

(a)(ii) a nucleic acid that encodes a fusion molecule comprising a sequence comprising a dCas9 molecule fused to a modulator of gene expression; and
(b)(ii) a nucleic acid that encodes a gRNA which targets the fusion molecule to a gene associated with the disorder,
   thereby treating the disorder.

In an embodiment, the disorder is selected from Table 2 or 3. In an embodiment, the gene is selected from Table 2 or 3.

In an embodiment, one or both of (a)(ii) and (b)(ii) are provided in an AAV vector.

In an embodiment, the fusion molecule comprises a fusion molecule described herein.

In an embodiment, the dCas9 molecule comprises a dCas9 molecule described herein.

In an embodiment, the modulator of gene expression comprises a modulator described herein.

In an embodiment, the gRNA comprises a gRNA sequence described herein.

In an embodiment, the disclosure features a method of treating a cardiovascular disease, comprising administering to a subject:
(a)(ii) a nucleic acid that encodes a fusion molecule comprising a sequence comprising a dCas9 molecule fused to a modulator of gene expression; and
(b)(ii) a nucleic acid that encodes a gRNA which targets the fusion molecule to a PCSK9 gene,
   thereby treating the cardiovascular disease.

In an embodiment, the fusion molecule comprises a fusion molecule described herein.

In an embodiment, the dCas9 molecule comprises a dCas9 molecule described herein.

In an embodiment, the modulator of gene expression comprises a modulator described herein.

In an embodiment, the dCas9 molecule is an S. aureus dCas9 molecule.

In an embodiment, the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.

In an embodiment, the gRNA comprises a gRNA sequence described herein.

In an embodiment, one or both of (a)(ii) and (b)(ii) are provided in an AAV vector.

In another aspect, the disclosure features:
(a)(i) a fusion molecule comprising a sequence comprising a dCas9 molecule fused to a modulator of gene expression; or (ii) a nucleic acid that encodes a fusion molecule comprising a sequence comprising a dCas9 molecule fused to a modulator of gene expression; and
(b)(i) a gRNA which targets the fusion molecule to a gene; or (ii) a nucleic acid that encodes a gRNA which targets the fusion molecule to the gene,
   for use in a method of modulating expression of the gene, in vivo, in a subject.

In an embodiment, the fusion molecule comprises a fusion molecule described herein.

In an embodiment, the dCas9 molecule comprises a dCas9 molecule described herein.

In an embodiment, the modulator of gene expression comprises a modulator described herein.

In an embodiment, the gRNA comprises a gRNA sequence described herein.

In an embodiment, the gene is a gene described herein.

In some embodiments, the method comprises a method described herein.

In another aspect, the disclosure features:
(a)(i) a fusion molecule comprising a sequence comprising a dCas9 molecule fused to a modulator of gene expression; or (ii) a nucleic acid that encodes a fusion molecule comprising a sequence comprising a dCas9 molecule fused to a modulator of gene expression; and
(b)(i) a gRNA which targets the fusion molecule to a gene; or (ii) a nucleic acid that encodes a gRNA which targets the fusion molecule to the gene,
   for use in a method of treating or preventing a disorder associated with the gene, in vivo, in a subject.

In an embodiment, the fusion molecule comprises a fusion molecule described herein.

In an embodiment, the dCas9 molecule comprises a dCas9 molecule described herein.

In an embodiment, the modulator of gene expression comprises a modulator described herein.

In an embodiment, the gRNA comprises a gRNA sequence described herein.

In an embodiment, the gene is a gene described herein.

In some embodiments, the disorder is a disorder described herein.

The present disclosure addresses these shortcomings by creating a modified programmable RNA-guided dCas9-based repressor for efficient packaging in AAV and in vivo gene regulation. This gene delivery system can be customized to target any endogenous gene by designing a new guide RNA molecule, enabling patent and stable gene repression in animal models and therapeutic use.

One aspect of the present disclosure provides a fusion protein comprising, consisting of, or consisting essentially of three heterologous polypeptide domains, wherein the first polypeptide domain comprises, consists of, or consists essentially of a dead Clustered Regularly Interspaced Short Palindromic Repeats associated (dCas) protein, the second polypeptide domain comprises, consists of, or consists essentially of a Kruppel-associated box (KRAB), and the polypeptide domain has an activity selected from the group consisting of transcription activation activity, transcription repression activity, transcription release factor activity, histone modification activity, nuclease activity, nucleic acid association activity, methylase activity, and demethylase activity.

Another aspect of the present disclosure provides a gene therapy construct comprising, consisting of, or consisting essentially of a polynucleotide encoding a fusion protein comprising three heterologous polypeptide domains, wherein the first polypeptide domain comprises, consists of, or consists essentially of a dead Clustered Regularly Interspaced Short Palindromic Repeats associated (dCas) protein, the second polypeptide domain comprises, consists of, or consists essentially of a Kruppel-associated box (KRAB), and the polypeptide domain has an activity selected from the group consisting of transcription activation activity, transcription repression activity, transcription release factor activity, histone modification activity, nuclease activity, nucleic acid association activity, methylase activity, and demethylase activity.

In some embodiments, the gene therapy construct comprises a vector system. In certain embodiments, the vector system comprises an AAV vector system.

In another embodiment, the gene therapy construct further comprises a first and second AAV inverted terminal repeat (ITR) sequence flanking the fusion protein.

Another aspect of the present disclosure provides a pharmaceutical composition comprising the gene therapy construct as described herein in a biocompatible pharmaceutical carrier.

In some embodiments, the Cas protein comprises Cas9.

In some embodiments, the gene therapy construct is designed for the targeted reduction of the PCSK9 gene. In some embodiments, the gene therapy construct is designed for the targeted reduction of the expression of the PCSK9 gene.

Another aspect of the present disclosure provides a method of suppressing the expression of a gene in a cell in vivo comprising, consisting of, or consisting essentially of administering to a cell a therapeutically effective amount of a gene therapy construct as described herein such that the gene expression is suppressed.

Another aspect of the present disclosure provides a method of suppressing a gene in vivo in a subject comprising, consisting of, or consisting essentially of administering to the subject a therapeutically effective amount of a gene therapy construct as described herein such that the gene is suppressed.

In some embodiments, the method is designed for the targeted reduction of the PCSK9 gene. In some embodiments, the method is designed for the targeted reduction of the expression of the PCSK9 gene.

Another aspect of the present disclosure provides a kit for the suppression of a gene in vivo comprising a gene therapy construct or pharmaceutical composition as described herein and instructions for use.

Yet another aspect of the present disclosure provides all that is described and illustrated herein.

### BRIEF DESCRIPTION OF THE DRAWINGS

The foregoing aspects and other features of the disclosure are explained in the following description, taken in connection with the accompanying drawings, herein:

FIGS. 1A-1D are graphs showing the adaptation of SaCas9 for transcriptional repression. FIG. 1A is a schematic graph showing introducing inactivating mutations D10A and N580A into the cleavage domains of SaCas9 to generate a nuclease-null dSaCas9 DNA-binding domain. FIG. 1B is a schematic graph showing a single lentiviral vector with puromycin resistance used to express dSaCas9-KRAB and a U6-gRNA cassette for in vitro testing of dSaCas9 repressors. FIGS. 1C and 1D are bar graphs showing that multiple gRNAs against the synthetic CAG promoter effected potent repression of mRNA by qPCR (FIG. 1C) and protein via luciferase bioluminescence (FIG. 1D) in primary mouse fibroblasts expressing a CAG-luciferase reporter cassette. * indicates p<0.05 by Student's t-test compared to non-treated (NT) controls (n=2 independent experiments).

FIGS. 2A and 2B are graphs showing the silencing of endogenous genes with the dSaCas9-KRAB repressor. In FIG. 2A, eight gRNAs were designed to target the skeletal muscle DNase-hypersensitivity peak upstream of the transcription start site in the endogenous mouse Acvr2b gene locus. FIG. 2B is a bar graph showing that several single gRNAs effected strong repression of Acvr2b when delivered with dSaCas9-KRAB, compared to no lentivirus (No LV) and dSaCas9-KRAB only (No gRNA) controls. * indicates p<0.05 by Student's t-test compared to No LV controls (n=2 independent experiments).

FIGS. 3A-3E are graphs showing the targeting of Acvr2b with AAV-dSaCas9-KRAB in vivo. FIG. 3A is a schematic showing a two-vector AAV9 expression system used to deliver dSaCas9-KRAB and Acvr2b gRNA intramuscularly to the right tibialis anterior muscle (TA) of adult wild-type mice. FIGS. 3B and 3D are bar graphs showing that dSaCas9 was efficiently expressed as measured by qPCR in the injected TA at 4 and 8 weeks, respectively, after injection. FIGS. 3C and 3E are bar graphs showing Acvr2b expression in the injected TA as assayed by qPCR at 4 and 8 weeks, respectively, post-AAV treatment. (n=3 mice, * indicates p<0.05 compared to PBS sham controls)

FIG. 4 is a bar graph showing the analysis of AAV-gRNA vector genome signal in intramuscularly injected mice. For PBS sham, AAV-dSaCas9-KRAB only, and AAV-dSaCas9-KRAB and AAV-Acvr2b-gRNA treated mice, the bars from left to right show the presence of the AAV-U6-gRNA vector, as measured by qPCR, in the liver, heart, right tibialis anterior (TA), left TA, right gastrocnemius (gastroc), and left gastroc, respectively.

FIGS. 5A-5D are graphs showing the silencing of endogenous genes in vivo with AAV-dSaCas9-KRAB. FIGS. 5A and 5C are bar graphs showing that intramuscular delivery of AAV9 expressing dSaCas9-KRAB results in efficient transgene expression in the liver and heart, respectively, 8 weeks after transduction in adult wild-type mice. FIGS. 5B and 5D are bar graphs showing that delivery of dSaCas9-KRAB with Acvr2b gRNA reduces target gene expression in the liver and heart, respectively, at 8 weeks after treatment. (n=3 mice, * indicates p<0.05 by Student's t-test compared to PBS sham controls)

FIG. 6 is a graph showing a restriction map of a lentiviral vector encoding S. aureus Cas9 KRAB-based repressor.

FIG. 7 is a graph showing a restriction map of an AAV vector encoding S. aureus Cas9 KRAB-based repressor.

FIG. 8 is a graph showing a restriction map of an AAV vector encoding S. aureus Cas9 U6-gRNA.

FIG. 9 is a graph showing a restriction map of an AAV vector encoding S. aureus Cas9 U6-gRNA.

FIGS. 10A-10C are schematics showing an AAV-based gene delivery system for CRISPR/Cas9-based synthetic repressors. In FIG. 10A, a nuclease-null S. aureus dCas9 DNA-binding domain was generated by introducing two catalytically inactivating mutations to the nuclease domains of Cas9. dCas9 derived from S. aureus was fused to a KRAB synthetic repressor to create a synthetic repressor for in vivo gene delivery. Dual vector (FIG. 10B) and single AAV vector (FIG. 10C) platforms were designed to efficiently express dCas9-KRAB and a custom guide RNA target molecule in vivo.

FIGS. 11A-11C are graphs showing targeted reduction of the PCSK9 gene in vivo with engineered synthetic repressors. FIG. 11A is a schematic showing vectors used for targeted reduction of PCSK9 expression. S. aureus dCas9-KRAB (dCas9-KRAB) was targeted to the mouse PCSK9 gene and delivered in a dual-vector AAV system intravenously in C57Bl/6 wild-type 7-week old mice. At 2 weeks post-systemic treatment, circulating PCSK9 (FIG. 11B) and total cholesterol levels (FIG. 11C) are significantly repressed in the serum compared to sham PBS-injected controls and dCas9-KRAB-treated controls without a guide RNA (* indicates p<0.05 by Student's t-test compared to PBS sham controls, n=4 mice per condition).

FIGS. 12A-12E are graphs showing results from a study in which mice were intravenously administered with PBS, or AAV vectors encoding dSaCas9-KRAB (dCK) alone, or low-dose dSaCas9-KRAB (dCK) and PCSK9 guide RNA (gRNA). FIG. 12A is a graph showing serum PCSK9 levels for the three treatment groups as measured by ELISA. FIG. 12B is a bar graph showing relative PCSK9 mRNA levels in the liver, as normalized to GAPDH mRNA levels, for the three treatment groups. FIG. 12C is a graph showing data from an RNA-Seq study comparing the RNA levels in the liver in the dSaCas9-KRAB and gRNA treatment group with those in the dSaCas9-KRAB alone treatment group. The dot representing PCSK9 RNA levels is labeled in the figure. FIGS. 12D and 12E are graphs showing the serum levels of total and LDL cholesterol for the three treatment groups as measured in a colorimetric assay.

FIGS. 13A-13F are graphs showing results from a study in which mice were intravenously administered with PBS, or AAV vectors encoding dSaCas9-KRAB (dCK) alone, PCSK9 guide RNA (gRNA) alone, or moderate-dose dSaCas9-KRAB (dCK) and PCSK9 guide RNA (gRNA). FIGS. 13A and 13B are graphs showing serum PCSK9 levels for the three treatment groups as measured by ELISA. In FIG. 13B, the serum PCSK9 levels are normalized to the levels at Day 0. FIGS. 13C and 13D are graphs showing total cholesterol levels in the serum for the three treatment groups as measured in a colorimetric assay. In FIG. 13D, the serum total cholesterol levels are normalized to the levels at Day 0. FIGS. 13E and 13F are graphs showing LDL cholesterol levels in the serum for the three treatment groups as measured in a colorimetric assay. In FIG. 13F, the serum LDL cholesterol levels are normalized to the levels at Day 0.

FIGS. 14A-14C are graphs showing results from a study in which mice were intravenously administered with PBS, moderate-dose, or high-dose of AAV vectors encoding dSaCas9-KRAB and PCSK9 gRNA. FIG. 14A is a graph showing serum PCSK9 levels for the three treatment groups as measured by ELISA. FIGS. 14B and 14C are graphs showing total cholesterol levels in the serum. FIG. 14D is a graph showing LDL cholesterol levels in the serum.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

### A. Definitions

As used herein, the term “coding sequence” or “encoding nucleic acid” means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The term “complement” or “complementary” as used herein with reference to a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The term “correcting”, “genome editing” and “restoring” refers to changing a mutant gene that encodes a mutant protein, a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

As used herein, the term “donor DNA”, “donor template” and “repair template” refers to a double-stranded DNA fragment or molecule that includes at least a portion of the gene of interest. The donor DNA may encode a full-functional protein or a partially-functional protein.

As used herein, the terms “frameshift” or “frameshift mutation” are used interchangeably and refer to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

As used herein, the term “functional” and “full-functional” describes a protein that has biological activity. A “functional gene” refers to a gene transcribed to mRNA, which is translated to a functional protein.

As used herein, the term “fusion protein” refers to a chimeric protein created through the covalent or non-covalent joining of two or more genes, directly or indirectly, that originally coded for separate proteins. In some embodiments, the translation of the fusion gene results in a single polypeptide with functional properties derived from each of the original proteins.

As used herein, the term “genetic construct” refers to the DNA or RNA molecules that comprise a nucleotide sequence that encodes a protein. The coding sequence includes initiation and termination signals operably linked to regulatory elements including a promoter and polyadenylation signal capable of directing expression in cells.

The term “Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, nonhomologous end joining may take place instead.

The term “genome editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

The term “identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the terms “mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

The term “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The term “normal gene” as used herein refers to a gene that has not undergone a change, such as a loss, gain, or exchange of genetic material. The normal gene undergoes normal gene transmission and gene expression.

The term “nuclease mediated NHEJ” as used herein refers to NHEJ that is initiated after a nuclease, such as a cas9, cuts double stranded DNA.

As used herein, the term “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially-functional” as used herein describes a protein that is encoded by a mutant gene and has less biological activity than a functional protein but more than a non-functional protein. In one embodiment, a partially-functional protein shows a biological activity that is less than 95%, 90%, 85%, 80%, 75%, 70%, 65%, 60%, 55%, 50%, 45%, 40%, 35%, or 30% of that of a corresponding functional protein.

The term “premature stop codon” or “out-of-frame stop codon” as used interchangeably herein refers to nonsense mutation in a sequence of DNA, which results in a stop codon at a location not normally found in the wild-type gene. A premature stop codon may cause a protein to be truncated or shorter compared to the full-length version of the protein.

The term “promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of a nucleic acid. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

The term “target gene” as used herein refers to any nucleotide sequence encoding a known or putative gene product. The target gene may be a mutated gene involved in a genetic disease or disorder.

The term “target region” as used herein refers to the region of the target gene to which the site-specific nuclease is designed to bind.

As used herein, the term “transgene” refers to a gene or genetic material containing a gene sequence that has been isolated from one organism and is introduced into a different organism. Alternatively, the term “transgene” also refers to a gene or genetic material that is chemically synthesized and introduced into an organism. This non-native segment of DNA may retain the ability to produce RNA or protein in the transgenic organism, or it may alter the normal function of the transgenic organism's genetic code. The introduction of a transgene has the potential to change the phenotype of an organism.

As used herein, the term “variant” when used with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982), incorporated herein by reference in its entirety. The hydropathic index of an amino acid is based on a consideration of its hydrophobicity and charge. It is known in the art that amino acids of similar hydropathic indexes may be substituted and still retain protein function. In one aspect, amino acids having hydropathic indexes of +2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, the term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, such as a DNA plasmid.

As used herein, the terms “gene transfer,” “gene delivery,” and “gene transduction” refer to methods or systems for reliably inserting a particular nucleotide sequence (e.g., DNA or RNA), fusion protein, polypeptide and the like into targeted cells. The vector may also comprise an adenovirus (AAV) vector. As used herein, the terms “adenoviral associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (e.g., functional ITRs) of the virus.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

The terms “adeno-associated virus inverted terminal repeats” or “AAV ITRs” refer to the palindromic regions found at each end of the AAV genome which function together in cis as origins of DNA replication and as packaging signals for the virus. For use in some embodiments of the present invention, flanking AAV ITRs are positioned 5′ and 3′ of one or more selected heterologous nucleotide sequences. Optionally, the ITRs together with the rep coding region or the Rep expression products provide for the integration of the selected sequences into the genome of a target cell.

As used herein, the term “AAV rep coding region” refers to the region of the AAV genome that encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Muzyczka (Muzyczka, Curr. Top. Microbiol. Immunol., 158:97-129 (1992)) and Kotin (Kotin, Hum. Gene Ther., 5:793-801 (1994)), incorporated herein by reference in their entirety, provide additional descriptions of the AAV rep coding region, as well as the cap coding region described below. Suitable homologues of the AAV rep coding region include the human herpesvirus 6 (HHV-6) rep gene which is also known to mediate AAV-2 DNA replication (Thomson el al., Virol., 204:304-311 (1994), incorporated herein by reference in its entirety).

As used herein, the term “AAV cap coding region” refers to the region of the AAV genome that encodes the capsid proteins VP1, VP2, and VP3, or functional homologues thereof. These cap expression products supply the packaging functions, which are collectively required for packaging the viral genome. In some embodiments, AAV2 Cap proteins may be used.

As used herein, the term “AAV helper function” refers to AAV coding regions capable of being expressed in a host cell to complement AAV viral functions missing from the AAV vector. Typically, the AAV helper functions include the AAV rep coding region and the AAV cap coding region. The helper functions may be contained in a “helper plasmid” or “helper construct.” An AAV helper construct as used herein, refers to a molecule that provides all or part of the elements necessary for AAV replication and packaging. Such AAV helper constructs may be a plasmid, virus or genes integrated into cell lines or into the cells of a subject. It may be provided as DNA, RNA, or protein. The elements do not have to be arranged co-linearly (i.e., in the same molecule). For example, rep78 and rep68 may be on different molecules. An “AAV helper construct” may be, for example, a vector containing AAV coding regions required to complement AAV viral functions missing from the AAV vector (e.g., the AAV rep coding region and the AAV cap coding region).

As used herein, the terms “accessory functions” and “accessory factors” refer to functions and factors that are required by AAV for replication, but are not provided by the AAV vector or AAV helper construct. Thus, these accessory functions and factors must be provided by the host cell, a virus (e.g., adenovirus or herpes simplex virus), or another expression vector that is co-expressed in the same cell. Generally, the E1, E2A, E4 and VA coding regions of adenovirus are used to supply the necessary accessory function required for AAV replication and packaging (Matsushita et al., Gene Therapy 5:938 (1998), incorporated herein by reference in its entirety).

Portions of the AAV genome have the capability of integrating into the DNA of cells to which it is introduced. As used herein, “integrate,” refers to portions of the genetic construct that become covalently bound to the genome of the cell to which it is administered, for example through the mechanism of action mediated by the AAV Rep protein and the AAV ITRs. For example, the AAV virus has been shown to integrate at 19q13.3-qter in the human genome. The minimal elements for AAV integration are the inverted terminal repeat (ITR) sequences and a functional Rep 78/68 protein. In some embodiments, the present invention incorporates the ITR sequences into a vector for integration to facilitate the integration of the transgene into the host cell genome for sustained transgene expression. The genetic transcript may also integrate into other chromosomes if the chromosomes contain the AAV integration site.

The predictability of insertion site reduces the danger of random insertional events into the cellular genome that may activate or inactivate host genes or interrupt coding sequences, consequences that limit the use of vectors whose integration is random, e.g., retroviruses. The Rep protein mediates the integration of the genetic construct containing the AAV ITRs and the transgene. The use of AAV is advantageous for its predictable integration site and because it has not been associated with human or non-human primate diseases, thus obviating many of the concerns that have been raised with virus-derived gene therapy vectors.

“Portion of the genetic construct integrates into a chromosome” refers to the portion of the genetic construct that will become covalently bound to the genome of the cell upon introduction of the genetic construct into the cell via administration of the gene therapy particle. The integration is mediated by the AAV ITRs flanking the transgene and the AAV Rep protein. Portions of the genetic construct that may be integrated into the genome include the transgene and the AAV ITRs.

The “transgene” may contain a transgenic sequence or a native or wild-type DNA sequence. The transgene may become part of the genome of the primate subject. A transgenic sequence can be partly or entirely species-heterologous, i.e., the transgenic sequence, or a portion thereof, can be from a species which is different from the cell into which it is introduced.

As used herein, the term “stably maintained” refers to characteristics of transgenic subject (e.g., a human or non-human primate) that maintain at least one of their transgenic elements (i.e., the element that is desired) through multiple generations of cells. For example, it is intended that the term encompass many cell division cycles of the originally transfected cell. The term “stable transfection” or “stably transfected” refers to the introduction and integration of foreign DNA into the genome of the cell. The term “stable transfectant” refers to a cell that has stably integrated foreign DNA into the genomic DNA.

As used herein, the terms “transgene encoding,” “nucleic acid molecule encoding,” “DNA sequence encoding,” and “DNA encoding” refer to the order or sequence of deoxyribonucleotides along a strand of deoxyribonucleic acid. The order of these deoxyribonucleotides may, for example, determine the order of amino acids along the polypeptide (protein) chain. The DNA sequence thus may code for the amino acid sequence.

As used herein, the term “wild type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants may be isolated, which are identified by the acquisition of altered characteristics when compared to the wild-type gene or gene product.

As used herein, the term “AAV virion,” “AAV particle,” or “AAV gene therapy particle,” “AAV gene therapy vector,” or “rAAV gene therapy vector” refers to a complete virus unit, such as a wt AAV virus particle (comprising a linear, single-stranded AAV nucleic acid genome associated with at least one AAV capsid protein coat). In this regard, single-stranded AAV nucleic acid molecules of either complementary sense (e.g., “sense” or “antisense” strands) can be packaged into any one AAV virion and both strands are equally infectious. Also included are infectious viral particles containing a heterologous DNA molecule of interest (e.g., CFTR or a biologically active portion thereof), which is flanked on both sides by AAV ITRs.

As used herein, the term “transfection” refers to the uptake of a foreign nucleic acid (e.g., DNA or RNA) by a cell. A cell has been “transfected” when an exogenous nucleic acid (DNA or RNA) has been introduced inside the cell membrane. A number of transfection techniques are generally known in the art (See, e.g., Graham et al., Virol., 52:456 (1973); Sambrook et al., Molecular Cloning, a Laboratory Manual, Cold Spring Harbor Laboratories, New York (1989); Davis et al., Basic Methods in Molecular Biology, Elsevier, (1986); and Chu et al., Gene 13:197 (1981), incorporated herein by reference in their entirety). Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell, which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest. The term includes the progeny of the parent cell, whether or not the progeny are identical in morphology or in genetic make-up to the original parent, so long as the selected nucleotide sequence is present. The recipient cell may be the cells of a subject to which the gene therapy particles and/or gene therapy vector has been administered.

As used herein, the term “recombinant DNA molecule” refers to a DNA molecule which is comprised of segments of DNA joined together by means of molecular biological techniques.

As used herein, the term “regulatory element” refers to a genetic element which can control the expression of nucleic acid sequences. For example, a promoter is a regulatory element that facilitates the initiation of transcription of an operably linked coding region. Other regulatory elements are splicing signals, polyadenylation signals, termination signals, etc.

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present.

Transcriptional control signals in eukaryotes generally comprise “promoter” and “enhancer” elements. Promoters and enhancers consist of short arrays of DNA sequences that interact specifically with cellular proteins involved in transcription (Maniatis et al., Science 236:1237 (1987), incorporated herein by reference in its entirety). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on the recipient cell type. Some eukaryotic promoters and enhancers have a broad host range while others are functional in a limited subset of cell types (See e.g., Voss et al., Trends Biochem. Sci., 11:287 (1986); and Maniatis et al., supra, for reviews, incorporated herein by reference in their entirety). For example, the SV40 early gene enhancer is very active in a variety of cell types from many mammalian species and has been used to express proteins in a broad range of mammalian cells (Dijkema et al, EMBO J. 4:761 (1985), incorporated herein by reference in its entirety). Promoter and enhancer elements derived from the human elongation factor 1-alpha gene (Uetsuki et al., J. Biol. Chem., 264:5791 (1989); Kim et al., Gene 91:217 (1990); and Mizushima and Nagata, Nucl. Acids. Res., 18:5322 (1990)), the long terminal repeats of the Rous sarcoma virus (Gorman et al., Proc. Natl. Acad. Sci. U.S.A. 79:6777 (1982)), and the human cytomegalovirus (Boshart et al., Cell 41:521 (1985)) are also of utility for expression of proteins in diverse mammalian cell types, incorporated herein by reference in their entirety. Promoters and enhancers can be found naturally, alone or together. For example, retroviral long terminal repeats comprise both promoter and enhancer elements. Generally promoters and enhancers act independently of the gene being transcribed or translated. Thus, the enhancer and promoter used can be “endogenous,” “exogenous,” or “heterologous” with respect to the gene to which they are operably linked. An “endogenous” enhancer/promoter is one which is naturally linked with a given gene in the genome. An “exogenous” or “heterologous” enhancer or promoter is one which is placed in juxtaposition to a gene by means of genetic manipulation (i.e., molecular biological techniques) such that transcription of that gene is directed by the linked enhancer/promoter.

As used herein, the term “CBA” promoter refers to a fusion of the chicken β-actin promoter and CMV immediate-early enhancer.

As used herein, the term “tissue specific” refers to regulatory elements or control sequences, such as a promoter, an enhancer, etc., wherein the expression of the nucleic acid sequence is substantially greater in a specific cell type(s) or tissue(s). In particularly preferred embodiments, the CB promoter (CB is the same as CBA defined above) displays good expression of human CFTR, rAAV5-CB-.DELTA.264CFTR, rAAV5-CB-.DELTA.27-264CFTR, or another biologically active portion of CFTR. It is not intended, however, that the present invention be limited to the CB promoter or to lung specific expression, as other tissue specific regulatory elements, or regulatory elements that display altered gene expression patterns, are encompassed within the invention.

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989), pp. 16.7-16.8, incorporated herein by reference in its entirety). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7, incorporated herein by reference in its entirety).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As defined herein, a “therapeutically effective amount” or “therapeutic effective dose” is an amount or dose of a fusion protein, polypeptide, nucleic acid, AAV particle(s), or virion(s) capable of producing sufficient amounts of a desired protein to modulate the activity of the protein in a desired manner, thus providing a palliative tool for clinical intervention. In some embodiments, a therapeutically effective amount or dose of a transfected fusion protein, polypeptide, nucleic acid, AAV particle(s), or virion(s) as described herein is enough to confer suppression of a gene targeted by the fusion protein/gene therapy construct.

As used herein, the term “treat”, e.g., a disorder, means that a subject (e.g., a human) who has a disorder, is at risk of having a disorder, and/or experiences a symptom of a disorder, will, in an embodiment, suffer a less severe symptom and/or will recover faster, when a fusion molecule or a nucleic acid that encodes the fusion molecule, and/or a gRNA or a nucleic acid that encodes the gRNA, e.g., as described herein, is administered than if the fusion molecule or a nucleic acid that encodes the fusion molecule, and/or the gRNA or a nucleic acid that encodes the gRNA, were never administered.

### B. CRISPR System

“Clustered Regularly Interspaced Short Palindromic Repeats” and “CRISPRs”, as used interchangeably herein, refer to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the single guide RNA (sgRNA), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the CRISPR RNA (crRNA), i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a trans-encoded small RNA (tracrRNA), which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes (S. pyogenes) Type II system naturally prefers to use an “NGG” sequence, where “N” can be any nucleotide, but also accepts other PAM sequences, such as “NAG” in engineered systems (Hsu et al, Nature Biotechnology (2013) doi: 10.1038/nbt.2647, incorporated herein by reference in its entirety). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi: 10.1038/nmeth.2681, incorporated herein by reference in its entirety).

### C. CRISPR/Cas9-Based System

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in genome editing and treating genetic diseases. The CRISPR/Cas9-based engineered systems may be designed to target any gene, including genes involved in a genetic disease, aging, tissue regeneration, or wound healing. The CRISPR/Cas9-based systems may include a Cas9 protein or Cas9 fusion protein and at least one gRNA. The Cas9 fusion protein may, for example, include a domain that has a different activity from what is endogenous to Cas9, such as a transactivation domain.

The target gene may be involved in differentiation of a cell or any other process in which activation of a gene may be desired, or may have a mutation such as a frameshift mutation or a nonsense mutation. If the target gene has a mutation that causes a premature stop codon, an aberrant splice acceptor site or an aberrant splice donor site, the CRISPR/Cas9-based system may be designed to recognize and bind a nucleotide sequence upstream or downstream from the premature stop codon, the aberrant splice acceptor site or the aberrant splice donor site. The CRISPR-Cas9-based system may also be used to disrupt normal gene splicing by targeting splice acceptors and donors to induce skipping of premature stop codons or restore a disrupted reading frame. The CRISPR/Cas9-based system may or may not mediate off-target changes to protein-coding regions of the genome. In some embodiments, the expression of the target gene is to be suppressed.

### D. Cas9

The CRISPR/Cas9-based system may include a Cas9 protein or a fragment thereof, a Cas9 fusion protein, a nucleic acid encoding a Cas9 protein or a fragment thereof, or a nucleic acid encoding a Cas9 fusion protein. As used herein, a “Cas9 molecule” may refer to a Cas9 protein, or a fragment thereof. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. Cas9 sequences and structures from different species are known in the art, see, e.g., Ferretti et al., Proc Natl Acad Sci USA. (2001); 98 (8): 4658-63; Deltcheva et al., Nature. 2011 Mar. 31; 471 (7340): 602-7; and Jinek et al., Science. (2012); 337 (6096): 816-21, incorporated herein by reference in their entirety. Exemplary S. pyogenes Cas9 sequence is available at the Uniprot database under accession number Q99ZW2. Exemplary Staphylococcus aureus (S. aureus) Cas9 sequence is available at the Uniprot database under accession number J7RUA5. Exemplary Cas9 sequences are also shown in Table 1.

S. pyogenes Cas9 is the most commonly studied Cas9 molecule. Notably, S. pyogenes Cas9 is quite large (the gene itself is over 4.1 Kb), making it challenging to be packed into certain delivery vectors. For example, Adeno-associated virus (AAV) vector has a packaging limit of 4.5 or 4.75 Kb. This means that Cas9 as well as regulatory elements such as a promoter and a transcription terminator all have to fit into the same viral vector. Constructs larger than 4.5 or 4.75 Kb will lead to significantly reduced virus production. One possibility is to use a functional fragment of S. pyogenes Cas9. Another possibility is to split Cas9 into its sub-portions (e.g., the N-terminal lobe and the C-terminal lobe of Cas9). Each sub-portion is expressed by a separate vector, and these sub-portions associate to form a functional Cas9. See, e.g., Chew et al., Nat Methods. 2016; 13:868-74; Truong et al., Nucleic Acids Res. 2015; 43:6450-6458; and Fine et al., Sci Rep. 2015; 5:10777, incorporated by reference herein in their entirety.

Alternatively, shorter Cas9 molecules from other species can be used in the compositions and methods disclosed herein, e.g., Cas9 molecules from Staphylococcus aureus, Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9). Exemplary Cas9 sequences from these species are also shown in Table 1. In certain embodiments, the present disclosure provides an AAV vector comprising a nucleotide encoding a Cas9 molecule from Streptococcus pyogenes, Staphylococcus aureus, Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9), or fragment thereof.

TABLE 1
Exemplary Cas9 amino acid sequences

| SEQ | | |
|---|---|---|
| ID NO: | Description | Sequence |
| 24 | S. pyogenes | MDKKYSIGLDIGTNSVGWAVITDEYKVPSKKFKVLGNTDRH |
| | serotype M1 | SIKKNLIGALLFDSGETAEATRLKRTARRRYTRRKNRICYLQ |
| | Cas9 (Q99ZW2) | EIFSNEMAKVDDSFFHRLEESFLVEEDKKHERHPIFGNIVDE |
| | | VAYHEKYPTIYHLRKKLVDSTDKADLRLIYLALAHMIKFRG |
| | | HFLIEGDLNPDNSDVDKLFIQLVQTYNQLFEENPINASGVDA |
| | | KAILSARLSKSRRLENLIAQLPGEKKNGLFGNLIALSLGLTPN |
| | | FKSNFDLAEDAKLQLSKDTYDDDLDNLLAQIGDQYADLFLA |
| | | AKNLSDAILLSDILRVNTEITKAPLSASMIKRYDEHHQDLTLL |
| | | KALVRQQLPEKYKEIFFDQSKNGYAGYIDGGASQEEFYKFIK |
| | | PILEKMDGTEELLVKLNREDLLRKQRTFDNGSIPHQIHLGEL |
| | | HAILRRQEDFYPFLKDNREKIEKILTFRIPYYVGPLARGNSRF |
| | | AWMTRKSEETITPWNFEEVVDKGASAQSFIERMTNFDKNLP |
| | | NEKVLPKHSLLYEYFTVYNELTKVKYVTEGMRKPAFLSGEQ |
| | | KKAIVDLLFKTNRKVTVKQLKEDYFKKIECFDSVEISGVEDR |
| | | FNASLGTYHDLLKIIKDKDFLDNEENEDILEDIVLTLTLFEDR |
| | | EMIEERLKTYAHLFDDKVMKQLKRRRYTGWGRLSRKLINGI |
| | | RDKQSGKTILDFLKSDGFANRNFMQLIHDDSLTFKEDIQKAQ |
| | | VSGQGDSLHEHIANLAGSPAIKKGILQTVKVVDELVKVMGR |
| | | HKPENIVIEMARENQTTQKGQKNSRERMKRIEEGIKELGSQI |
| | | LKEHPVENTQLQNEKLYLYYLQNGRDMYVDQELDINRLSD |
| | | YDVDHIVPQSFLKDDSIDNKVLTRSDKNRGKSDNVPSEEVV |
| | | KKMKNYWRQLLNAKLITQRKFDNLTKAERGGLSELDKAGF |
| | | IKRQLVETRQITKHVAQILDSRMNTKYDENDKLIREVKVITL |
| | | KSKLVSDFRKDFQFYKVREINNYHHAHDAYLNAVVGTALI |
| | | KKYPKLESEFVYGDYKVYDVRKMIAKSEQEIGKATAKYFFY |
| | | SNIMNFFKTEITLANGEIRKRPLIETNGETGEIVWDKGRDFAT |
| | | VRKVLSMPQVNIVKKTEVQTGGFSKESILPKRNSDKLIARKK |
| | | DWDPKKYGGFDSPTVAYSVLVVAKVEKGKSKKLKSVKELL |
| | | GITIMERSSFEKNPIDFLEAKGYKEVKKDLIIKLPKYSLFELEN |
| | | GRKRMLASAGELQKGNELALPSKYVNFLYLASHYEKLKGS |
| | | PEDNEQKQLFVEQHKHYLDEIIEQISEFSKRVILADANLDKV |
| | | LSAYNKHRDKPIREQAENIIHLFTLTNLGAPAAFKYFDTTIDR |
| | | KRYTSTKEVLDATLIHQSITGLYETRIDLSQLGGD |
| 25 | S. aureus Cas9 | MKRNYILGLDIGITSVGYGIIDYETRDVIDAGVRLFKEANVE |
| | (J7RUA5) | NNEGRRSKRGARRLKRRRRHRIQRVKKLLFDYNLLTDHSEL |
| | | SGINPYEARVKGLSQKLSEEEFSAALLHLAKRRGVHNVNEV |
| | | EEDTGNELSTKEQISRNSKALEEKYVAELQLERLKKDGEVR |
| | | GSINRFKTSDYVKEAKQLLKVQKAYHQLDQSFIDTYIDLLET |
| | | RRTYYEGPGEGSPFGWKDIKEWYEMLMGHCTYFPEELRSV |
| | | KYAYNADLYNALNDLNNLVITRDENEKLEYYEKFQIIENVF |
| | | KQKKKPTLKQIAKEILVNEEDIKGYRVTSTGKPEFTNLKVYH |
| | | DIKDITARKEIIENAELLDQIAKILTIYQSSEDIQEELTNLNSEL |
| | | TQEEIEQISNLKGYTGTHNLSLKAINLILDELWHTNDNQIAIF |
| | | NRLKLVPKKVDLSQQKEIPTTLVDDFILSPVVKRSFIQSIKVI |
| | | NAIIKKYGLPNDIIIELAREKNSKDAQKMINEMQKRNRQTNE |
| | | RIEEIIRTTGKENAKYLIEKIKLHDMQEGKCLYSLEAIPLEDL |
| | | LNNPFNYEVDHIIPRSVSFDNSFNNKVLVKQEENSKKGNRTP |
| | | FQYLSSSDSKISYETFKKHILNLAKGKGRISKTKKEYLLEERD |
| | | INRFSVQKDFINRNLVDTRYATRGLMNLLRSYFRVNNLDVK |
| | | VKSINGGFTSFLRRKWKFKKERNKGYKHHAEDALIIANADFI |
| | | FKEWKKLDKAKKVMENQMFEEKQAESMPEIETEQEYKEIFI |
| | | TPHQIKHIKDFKDYKYSHRVDKKPNRELINDTLYSTRKDDK |
| | | GNTLIVNNLNGLYDKDNDKLKKLINKSPEKLLMYHHDPQT |
| | | YQKLKLIMEQYGDEKNPLYKYYEETGNYLTKYSKKDNGPV |
| | | IKKIKYYGNKLNAHLDITDDYPNSRNKVVKLSLKPYRFDVY |
| | | LDNGVYKFVTVKNLDVIKKENYYEVNSKCYEEAKKLKKIS |
| | | NQAEFIASFYNNDLIKINGELYRVIGVNNDLLNRIEVNMIDIT |
| | | YREYLENMNDKRPPRIIKTIASKTQSIKKYSTDILGNLYEVKS |
| | | KKHPQIIKKG |
| 26 | Eubacterium | MGYTVGLDIGVASVGVAVLDENDNIVEAVSNIFDEADTSNN |
| | ventriosum | KVRRTLREGRRTKRRQKTRIEDFKQLWETSGYIIPHKLHLNII |
| | Cas9 | ELRNKGLTELLSLDELYCVLLSMLKHRGISYLEDADDGEKG |
| | (A5Z395) | NAYKKGLAFNEKQLKEKMPCEIQLERMKKYGKYHGEFIIEI |
| | | NDEKEYQSNVFTTKAYKKELEKIFETQRCNGNKINTKFIKKY |
| | | MEIYERKREYYIGPGNEKSRTDYGIYTTRTDEEGNFIDEKNIF |
| | | GKLIGKCSVYPEEYRASSASYTAQEFNLLNDLNNLKINNEKL |
| | | TEFQKKEIVEIIKDASSVNMRKIIKKVIDEDIEQYSGARIDKK |
| | | GKEIYHTFEIYRKLKKELKTINVDIDSFTREELDKTMDILTLN |
| | | TERESIVKAFDEQKFVYEENLIKKLIEFRKNNQRLFSGWHSF |
| | | SYKAMLQLIPVMYKEPKEQMQLLTEMNVFKSKKEKYVNY |
| | | KYIPENEVVKEIYNPVVVKSIRTTVKILNALIKKYGYPESVVI |
| | | EMPRDKNSDDEKEKIDMNQKKNQEEYEKILNKIYDEKGIEIT |
| | | NKDYKKQKKLVLKLKLWNEQEGLCLYSGKKIAIEDLLNHP |
| | | EFFEIDHIIPKSISLDDSRSNKVLVYKTENSIKENDTPYHYLTR |
| | | INGKWGFDEYKANVLELRRRGKIDDKKVNNLLCMEDITKID |
| | | VVKGFINRNLNDTRYASRVVLNEMQSFFESRKYCNTKVKVI |
| | | RGSLTYQMRQDLHLKKNREESYSHHAVDAMLIAFSQKGYE |
| | | AYRKIQKDCYDFETGEILDKEKWNKYIDDDEFDDILYKERM |
| | | NEIRKKIIEAEEKVKYNYKIDKKCNRGLCNQTIYGTREKDGK |
| | | IHKISSYNIYDDKECNSLKKMINSGKGSDLLMYNNDPKTYR |
| | | DMLKILETYSSEKNPFVAYNKETGDYFRKYSKNHNGPKVEK |
| | | VKYYSGQINSCIDISHKYGHAKNSKKVVLVSLNPYRTDVYY |
| | | DNDTGKYYLVGVKYNHIKCVGNKYVIDSETYNELLRKEGV |
| | | LNSDENLEDLNSKNITYKFSLYKNDIIQYEKGGEYYTERFLS |
| | | RIKEQKNLIETKPINKPNFQRKNKKGEWENTRNQIALAKTK |
| | | YVGKLVTDVLGNCYIVNMEKFSLVVDK |
| 27 | Azospirillum | MARPAFRAPRREHVNGWTPDPHRISKPFFILVSWHLLSRVVI |
| | (strain B510) | DSSSGCFPGTSRDHTDKFAEWECAVQPYRLSFDLGTNSIGW |
| | Cas9 | GLLNLDRQGKPREIRALGSRIFSDGRDPQDKASLAVARRLA |
| | (D3NT09) | RQMRRRRDRYLTRRTRLMGALVRFGLMPADPAARKRLEV |
| | | AVDPYLARERATRERLEPFEIGRALFHLNQRRGYKPVRTAT |
| | | KPDEEAGKVKEAVERLEAAIAAAGAPTLGAWFAWRKTRGE |
| | | TLRARLAGKGKEAAYPFYPARRMLEAEFDTLWAEQARHHP |
| | | DLLTAEAREILRHRIFHQRPLKPPPVGRCTLYPDDGRAPRAL |
| | | PSAQRLRLFQELASLRVIHLDLSERPLTPAERDRIVAFVQGRP |
| | | PKAGRKPGKVQKSVPFEKLRGLLELPPGTGFSLESDKRPELL |
| | | GDETGARIAPAFGPGWTALPLEEQDALVELLLTEAEPERAIA |
| | | ALTARWALDEATAAKLAGATLPDFHGRYGRRAVAELLPVL |
| | | ERETRGDPDGRVRPIRLDEAVKLLRGGKDHSDFSREGALLD |
| | | ALPYYGAVLERHVAFGTGNPADPEEKRVGRVANPTVHIAL |
| | | NQLRHLVNAILARHGRPEEIVIELARDLKRSAEDRRREDKRQ |
| | | ADNQKRNEERKRLILSLGERPTPRNLLKLRLWEEQGPVENR |
| | | RCPYSGETISMRMLLSEQVDIDHILPFSVSLDDSAANKVVCL |
| | | REANRIKRNRSPWEAFGHDSERWAGILARAEALPKNKRWR |
| | | FAPDALEKLEGEGGLRARHLNDTRHLSRLAVEYLRCVCPKV |
| | | RVSPGRLTALLRRRWGIDAILAEADGPPPEVPAETLDPSPAE |
| | | KNRADHRHHALDAVVIGCIDRSMVQRVQLAAASAEREAAA |
| | | REDNIRRVLEGFKEEPWDGFRAELERRARTIVVSHRPEHGIG |
| | | GALHKETAYGPVDPPEEGFNLVVRKPIDGLSKDEINSVRDPR |
| | | LRRALIDRLAIRRRDANDPATALAKAAEDLAAQPASRGIRR |
| | | VRVLKKESNPIRVEHGGNPSGPRSGGPFHKLLLAGEVHHVD |
| | | VALRADGRRWVGHWVTLFEAHGGRGADGAAAPPRLGDGE |
| | | RFLMRLHKGDCLKLEHKGRVRVMQVVKLEPSSNSVVVVEP |
| | | HQVKTDRSKHVKISCDQLRARGARRVTVDPLGRVRVHAPG |
| | | ARVGIGGDAGRTAMEPAEDIS |
| 28 | Gluconacetobacter | MGENMIDESLTFGIDLGIGSCGWAVLRRPSAFGRKGVIEGM |
| | diazotrophicus | GSWCFDVPETSKERTPTNQIRRSNRLLRRVIRRRRNRMAAIR |
| | (strain ATCC | RLLHAAGLLPSTDSDALKRPGHDPWELRARGLDKPLKPVEF |
| | 49037) Cas9 | AVVLGHIAKRRGFKSAAKRKATNISSDDKKMLTALEATRER |
| | (A9HKP2) | LGRYRTVGEMFARDPDFASRRRNREGKYDRTTARDDLEHE |
| | | VHALFAAQRRLGQGFASPELEEAFTASAFHQRPMQDSERLV |
| | | GFCPFERTEKRAAKLTPSFERFRLLARLLNLRITTPDGERPLT |
| | | VDEIALVTRDLGKTAKLSIKRVRTLIGLEDNQRFTTIRPEDED |
| | | RDIVARTGGAMTGTATLRKALGEALWTDMQERPEQLDAIV |
| | | QVLSFFEANETITEKLREIGLTLAVLDVLLTALDAGVFAKFK |
| | | GAAHISTKAARNLLPHLEQGRRYDEACTMAGYDHAASRLS |
| | | HHGQIVAKTQFNALVTEIGESIANPIARKALIEGLKQIWAMR |
| | | NHWGLPGSIHVELARDVGNSIEKRREIEKHIEKNTALRARER |
| | | REVHDLLDLEDVNGDTLLRYRLWKEQGGKCLYTGKAIHIR |
| | | QIAATDNSVQVDHILPWSRFGDDSFNNKTLCLASANQQKKR |
| | | STPYEWLSGQTGDAWNAFVQRIETNKELRGFKKRNYLLKN |
| | | AKEAEEKFRSRNLNDTRYAARLFAEAVKLLYAFGERQEKG |
| | | GNRRVFTRPGALTAALRQAWGVESLKKQDGKRINDDRHHA |
| | | LDALTVAAVDEAEIQRLTKSFHEWEQQGLGRPLRRVEPPWE |
| | | SFRADVEATYPEVFVARPERRRARGEGHAATIRQVKERECT |
| | | PIVFERKAVSSLKEADLERIKDGERNEAIVEAIRSWIATGRPA |
| | | DAPPRSPRGDIITKIRLATTIKAAVPVRGGTAGRGEMVRADV |
| | | FSKPNRRGKDEWYLVPVYPHQIMNRKAWPKPPMRSIVANK |
| | | DEDEWTEVGPEHQFRFSLYPRSNIEIIRPSGEVIEGYFVGLHR |
| | | NTGALTISAHNDPKSIHSGIGTKTLLAISKYQVDRFGRKSPVR |
| | | KEVRTWHGEACISPTPPG |
| 29 | Neisseria | MAAFKPNPMNYILGLDIGIASVGWAIVEIDEEENPIRLIDLGV |
| | cinerea Cas9 | RVFERAEVPKTGDSLAAARRLARSVRRLTRRRAHRLLRARR |
| | (D0W2Z9) | LLKREGVLQAADFDENGLIKSLPNTPWQLRAAALDRKLTPL |
| | | EWSAVLLHLIKHRGYLSQRKNEGETADKELGALLKGVADN |
| | | THALQTGDFRTPAELALNKFEKESGHIRNQRGDYSHTFNRK |
| | | DLQAELNLLFEKQKEFGNPHVSDGLKEGIETLLMTQRPALS |
| | | GDAVQKMLGHCTFEPTEPKAAKNTYTAERFVWLTKLNNLR |
| | | ILEQGSERPLTDTERATLMDEPYRKSKLTYAQARKLLDLDD |
| | | TAFFKGLRYGKDNAEASTLMEMKAYHAISRALEKEGLKDK |
| | | KSPLNLSPELQDEIGTAFSLFKTDEDITGRLKDRVQPEILEAL |
| | | LKHISFDKFVQISLKALRRIVPLMEQGNRYDEACTEIYGDHY |
| | | GKKNTEEKIYLPPIPADEIRNPVVLRALSQARKVINGVVRRY |
| | | GSPARIHIETAREVGKSFKDRKEIEKRQEENRKDREKSAAKF |
| | | REYFPNFVGEPKSKDILKLRLYEQQHGKCLYSGKEINLGRLN |
| | | EKGYVEIDHALPFSRTWDDSFNNKVLALGSENQNKGNQTP |
| | | YEYFNGKDNSREWQEFKARVETSRFPRSKKQRILLQKFDED |
| | | GFKERNLNDTRYINRFLCQFVADHMLLTGKGKRRVFASNG |
| | | QITNLLRGFWGLRKVRAENDRHHALDAVVVACSTIAMQQK |
| | | ITRFVRYKEMNAFDGKTIDKETGEVLHQKAHFPQPWEFFAQ |
| | | EVMIRVFGKPDGKPEFEEADTPEKLRTLLAEKLSSRPEAVHK |
| | | YVTPLFISRAPNRKMSGQGHMETVKSAKRLDEGISVLRVPL |
| | | TQLKLKDLEKMVNREREPKLYEALKARLEAHKDDPAKAFA |
| | | EPFYKYDKAGNRTQQVKAVRVEQVQKTGVWVHNHNGIAD |
| | | NATIVRVDVFEKGGKYYLVPIYSWQVAKGILPDRAVVQGK |
| | | DEEDWTVMDDSFEFKFVLYANDLIKLTAKKNEFLGYFVSLN |
| | | RATGAIDIRTHDTDSTKGKNGIFQSVGVKTALSFQKYQIDEL |
| | | GKEIRPCRLKKRPPVR |
| 30 | Roseburia | MRENGSDERRRNMDEKMDYRIGLDIGIASVGWAVLQNNSD |
| | intestinalis | DEPVRIVDLGVRIFDTAEIPKTGESLAGPRRAARTTRRRLRR |
| | Cas9 | RKHRLDRIKWLFENQGLINIDDFLKRYNMAGLPDVYQLRYE |
| | (C7G697) | ALDRKLTDEELAQVLLHIAKHRGFRSTRKAETAAKENGAVL |
| | | KATDENQKRMQEKGYRTVGEMIYLDEAFRTGCSWSEKGYI |
| | | LTPRNKAENYQHTMLRAMLVEEVKEIFSSQRRLGNEKATEE |
| | | LEEKYLEIMTSQRSFDLGPGMQPDGKPSPYAMEGFSDRVGK |
| | | CTFLGDQGELRGAKGTYTAEYFVALQKINHTKLVNQDGET |
| | | RNFTEEERRALTLLLFTQKEVKYAAVRKKLGLPEDILFYNLN |
| | | YKKAATKEEQQKENQNTEKAKFIGMPYYHDYKKCLEERVK |
| | | YLTENEVRDLFDEIGMILTCYKNDDSRTERLAKLGLVPIEME |
| | | GLLAYTPTKFQHLSMKAMRNIIPFLEKGMTYDKACEEAGYD |
| | | FKADSKGTKQKLLTGENVNQTINEITNPVVKRSVSQTVKVIN |
| | | AIIRTYGSPQAINIELAREMSKTFEERRKIKGDMEKRQKNNE |
| | | DVKKQIQELGKLSPTGQDILKYRLWQEQQGICMYSGKTIPLE |
| | | ELFKPGYDIDHILPYSITFDDSFRNKVLVTSQENRQKGNRTP |
| | | YEYMGNDEQRWNEFETRVKTTIRDYKKQQKLLKKHFSEEE |
| | | RSEFKERNLTDTKYITTVIYNMIRQNLEMAPLNRPEKKKQV |
| | | RAVNGAITAYLRKRWGLPQKNRETDTHHAMDAVVIACCTD |
| | | GMIQKISRYTKVRERCYSKGTEFVDAETGEIFRPEDYSRAEW |
| | | DEIFGVHIPKPWETFRAELDVRMGDDPKGFLDTHSDVALEL |
| | | DYPEYIYENLRPIFVSRMPNHKVTGAAHADTIRSPRHFKDEG |
| | | IVLTKTALTDLKLDKDGEIDGYYNPQSDLLLYEALKKQLLL |
| | | YGNDAKKAFAQDFHKPKADGTEGPVVRKVKIQKKQTMGV |
| | | FVDSGNGIAENGGMVRIDVFRVNGKYYFVPVYTADVVKKV |
| | | LPNRASTAHKPYGEWKVMEDKDFLFSLYSRDLIHIKSKKDIP |
| | | IKMVNGGMEGIKETYAYYIGADISAANIQGIAHDSRYKFRGL |
| | | GIQSLDVLEKCQIDVLGHVSVVRSEKRMGFS |
| 31 | Parvibaculum | MERIFGFDIGTTSIGFSVIDYSSTQSAGNIQRLGVRIFPEARDP |
| | lavamentivorans | DGTPLNQQRRQKRMMRRQLRRRRIRRKALNETLHEAGFLP |
| | (strain DS- | AYGSADWPVVMADEPYELRRRGLEEGLSAYEFGRAIYHLA |
| | 1) Cas9 | QHRHFKGRELEESDTPDPDVDDEKEAANERAATLKALKNE |
| | (A7HP89) | QTTLGAWLARRPPSDRKRGIHAHRNVVAEEFERLWEVQSK |
| | | FHPALKSEEMRARISDTIFAQRPVFWRKNTLGECRFMPGEPL |
| | | CPKGSWLSQQRRMLEKLNNLAIAGGNARPLDAEERDAILSK |
| | | LQQQASMSWPGVRSALKALYKQRGEPGAEKSLKFNLELGG |
| | | ESKLLGNALEAKLADMFGPDWPAHPRKQEIRHAVHERLWA |
| | | ADYGETPDKKRVIILSEKDRKAHREAAANSFVADFGITGEQ |
| | | AAQLQALKLPTGWEPYSIPALNLFLAELEKGERFGALVNGP |
| | | DWEGWRRTNFPHRNQPTGEILDKLPSPASKEERERISQLRNP |
| | | TVVRTQNELRKVVNNLIGLYGKPDRIRIEVGRDVGKSKRER |
| | | EEIQSGIRRNEKQRKKATEDLIKNGIANPSRDDVEKWILWKE |
| | | GQERCPYTGDQIGFNALFREGRYEVEHIWPRSRSFDNSPRNK |
| | | TLCRKDVNIEKGNRMPFEAFGHDEDRWSAIQIRLQGMVSAK |
| | | GGTGMSPGKVKRFLAKTMPEDFAARQLNDTRYAAKQILAQ |
| | | LKRLWPDMGPEAPVKVEAVTGQVTAQLRKLWTLNNILADD |
| | | GEKTRADHRHHAIDALTVACTHPGMTNKLSRYWQLRDDPR |
| | | AEKPALTPPWDTIRADAEKAVSEIVVSHRVRKKVSGPLHKE |
| | | TTYGDTGTDIKTKSGTYRQFVTRKKIESLSKGELDEIRDPRIK |
| | | EIVAAHVAGRGGDPKKAFPPYPCVSPGGPEIRKVRLTSKQQL |
| | | NLMAQTGNGYADLGSNHHIAIYRLPDGKADFEIVSLFDASR |
| | | RLAQRNPIVQRTRADGASFVMSLAAGEAIMIPEGSKKGIWIV |
| | | QGVWASGQVVLERDTDADHSTTTRPMPNPILKDDAKKVSI |
| | | DPIGRVRPSND |
| 32 | Nitratifractor | MKKILGVDLGITSFGYAILQETGKDLYRCLDNSVVMRNNPY |
| | salsuginis | DEKSGESSQSIRSTQKSMRRLIEKRKKRIRCVAQTMERYGIL |
| | (strain DSM | DYSETMKINDPKNNPIKNRWQLRAVDAWKRPLSPQELFAIF |
| | 16511) Cas9 | AHMAKHRGYKSIATEDLIYELELELGLNDPEKESEKKADER |
| | (E6WZS9) | RQVYNALRHLEELRKKYGGETIAQTIHRAVEAGDLRSYRNH |
| | | DDYEKMIRREDIEEEIEKVLLRQAELGALGLPEEQVSELIDEL |
| | | KACITDQEMPTIDESLFGKCTFYKDELAAPAYSYLYDLYRL |
| | | YKKLADLNIDGYEVTQEDREKVIEWVEKKIAQGKNLKKITH |
| | | KDLRKILGLAPEQKIFGVEDERIVKGKKEPRTFVPFFFLADIA |
| | | KFKELFASIQKHPDALQIFRELAEILQRSKTPQEALDRLRAL |
| | | MAGKGIDTDDRELLELFKNKRSGTRELSHRYILEALPLFLEG |
| | | YDEKEVQRILGFDDREDYSRYPKSLRHLHLREGNLFEKEEN |
| | | PINNHAVKSLASWALGLIADLSWRYGPFDEIILETTRDALPE |
| | | KIRKEIDKAMREREKALDKIIGKYKKEFPSIDKRLARKIQLW |
| | | ERQKGLDLYSGKVINLSQLLDGSADIEHIVPQSLGGLSTDYN |
| | | TIVTLKSVNAAKGNRLPGDWLAGNPDYRERIGMLSEKGLID |
| | | WKKRKNLLAQSLDEIYTENTHSKGIRATSYLEALVAQVLKR |
| | | YYPFPDPELRKNGIGVRMIPGKVTSKTRSLLGIKSKSRETNFH |
| | | HAEDALILSTLTRGWQNRLHRMLRDNYGKSEAELKELWKK |
| | | YMPHIEGLTLADYIDEAFRRFMSKGEESLFYRDMFDTIRSISY |
| | | WVDKKPLSASSHKETVYSSRHEVPTLRKNILEAFDSLNVIKD |
| | | RHKLTTEEFMKRYDKEIRQKLWLHRIGNTNDESYRAVEERA |
| | | TQIAQILTRYQLMDAQNDKEIDEKFQQALKELITSPIEVTGKL |
| | | LRKMRFVYDKLNAMQIDRGLVETDKNMLGIHISKGPNEKLI |
| | | FRRMDVNNAHELQKERSGILCYLNEMLFIFNKKGLIHYGCL |
| | | RSYLEKGQGSKYIALFNPRFPANPKAQPSKFTSDSKIKQVGI |
| | | GSATGIIKAHLDLDGHVRSYEVFGTLPEGSIEWFKEESGYGR |
| | | VEDDPHH |
| 33 | Campylobacter | MRILGFDIGINSIGWAFVENDELKDCGVRIFTKAENPKNKES |
| | lari Cas9 | LALPRRNARSSRRRLKRRKARLIAIKRILAKELKLNYKDYVA |
| | (G1UFN3) | ADGELPKAYEGSLASVYELRYKALTQNLETKDLARVILHIA |
| | | KHRGYMNKNEKKSNDAKKGKILSALKNNALKLENYQSVG |
| | | EYFYKEFFQKYKKNTKNFIKIRNTKDNYNNCVLSSDLEKEL |
| | | KLILEKQKEFGYNYSEDFINEILKVAFFQRPLKDFSHLVGAC |
| | | TFFEEEKRACKNSYSAWEFVALTKIINEIKSLEKISGEIVPTQT |
| | | INEVLNLILDKGSITYKKFRSCINLHESISFKSLKYDKENAEN |
| | | AKLIDFRKLVEFKKALGVHSLSRQELDQISTHITLIKDNVKL |
| | | KTVLEKYNLSNEQINNLLEIEFNDYINLSFKALGMILPLMRE |
| | | GKRYDEACEIANLKPKTVDEKKDFLPAFCDSIFAHELSNPVV |
| | | NRAISEYRKVLNALLKKYGKVHKIHLELARDVGLSKKAREK |
| | | IEKEQKENQAVNAWALKECENIGLKASAKNILKLKLWKEQ |
| | | KEICIYSGNKISIEHLKDEKALEVDHIYPYSRSFDDSFINKVLV |
| | | FTKENQEKLNKTPFEAFGKNIEKWSKIQTLAQNLPYKKKNKI |
| | | LDENFKDKQQEDFISRNLNDTRYIATLIAKYTKEYLNFLLLS |
| | | ENENANLKSGEKGSKIHVQTISGMLTSVLRHTWGFDKKDRN |
| | | NHLHHALDAIIVAYSTNSIIKAFSDFRKNQELLKARFYAKEL |
| | | TSDNYKHQVKFFEPFKSFREKILSKIDEIFVSKPPRKRARRAL |
| | | HKDTFHSENKIIDKCSYNSKEGLQIALSCGRVRKIGTKYVEN |
| | | DTIVRVDIFKKQNKFYAIPIYAMDFALGILPNKIVITGKDKNN |
| | | NPKQWQTIDESYEFCFSLYKNDLILLQKKNMQEPEFAYYND |
| | | FSISTSSICVEKHDNKFENLTSNQKLLFSNAKEGSVKVESLGI |
| | | QNLKVFEKYIITPLGDKIKADFQPRENISLKTSKKYGLR |

In one embodiment, Cas9 comprises one or more of the following domains: a Rec1 domain, a Rec2 domain, a bridge helix domain, a PAM interacting domain, an HNH nuclease domain, and a RuvC nuclease domain. Without wishing to be bound by theory, the Rec1 domain is responsible for binding guide RNA. The arginine-rich bridge helix domain plays an important role in initiating cleavage activity upon binding of target DNA. The PAM-Interacting domain confers PAM specificity and is therefore responsible for initiating binding to target DNA. The HNH and RuvC domains are nuclease domains that cut single-stranded DNA complementary and noncomplementary to the guide RNA, respectively. See, e.g., Nishimasu et al., Cell (2014) 156:935-49; Anders et al., Nature (2014) 513:569-73; Jinek et al., Science (2014) 343:1247997; Sternberg et al., Nature (2014) 507:62-7, incorporated by reference herein in their entirety.

### E. dCas9

The Cas9 protein may be mutated so that the nuclease activity is inactivated. An inactivated Cas9 protein from S. pyogenes (iCas9, also referred to as “dCas9”) with no endonuclease activity has been recently targeted to genes in bacteria, yeast, and human cells by gRNA to silence gene expression through steric hindrance. As used herein, a “dCas molecule” may refer to a dCas protein, or a fragment thereof. As used herein, a “dCas9 molecule” may refer to a dCas9 protein, or a fragment thereof. As used herein, the terms “iCas” and “dCas” are used interchangeably and refer to a catalytically inactive CRISPR associated protein. In one embodiment, the dCas molecule comprises one or more mutations in a DNA-cleavage domain. In one embodiment, the dCas molecule comprises one or more mutations in the RuvC or HNH domain. In one embodiment, the dCas molecule comprises one or more mutations in both the RuvC and HNH domain. In one embodiment, the dCas molecule is a fragment of a wild-type Cas molecule. In one embodiment, the dCas molecule comprises a functional domain from a wild-type Cas molecule, wherein the functional domain is chosen from a Rec1 domain, a bridge helix domain, or a PAM interacting domain. In one embodiment, the nuclease activity of the dCas molecule is reduced by at least 40%, 45%, 50%, 55%, 60%, 65%, 70%, 75%, 80%, 85%, 90%, 95%, 99% compared to that of a corresponding wild type Cas molecule.

Suitable dCas molecule can be derived from a wild type Cas molecule. The Cas molecule can be from a type I, type II, or type III CRISPR-Cas systems. In one embodiment, suitable dCas molecules can be derived from a Cas1, Cas2, Cas3, Cas4, Cas5, Cas6, Cas7, Cas8, Cas9, or Cas10 molecule. In one embodiment, the dCas molecule is derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 sgRNA and DNA binding activity. In one embodiment, the two point mutations within the RuvC and HNH active sites are D10A and H840A mutations of the S. pyogenes Cas9 molecule. Alternatively, D10 and H840 of the S. pyogenes Cas9 molecule can be deleted to abolish the Cas9 nuclease activity while retaining its sgRNA and DNA binding activity. In one embodiment, the two point mutations within the RuvC and HNH active sites are D10A and N580A mutations of the S. aureus Cas9 molecule. In one embodiment, the dCas molecule is an S. aureus dCas9 molecule comprising a mutation at D10 and/or N580, numbered according to SEQ ID NO: 25. In one embodiment, the dCas molecule is an S. aureus dCas9 molecule comprising D10A and/or N580A mutations, numbered according to SEQ ID NO: 25. In one embodiment, the dCas molecule is an S. aureus dCas9 molecule comprising the amino acid sequence of SEQ ID NO: 35 or 36, a sequence substantially identical (e.g., at least 80%, 85%, 90%, 92%, 95%, 97%, 98%, 99% or higher identical) to SEQ ID NO: 35 or 36, or a sequence having one, two, three, four, five or more changes, e.g., amino acid substitutions, insertions, or deletions, relative to SEQ ID NO: 35 or 36, or any fragment thereof.

(exemplary S. aureus dCas9)
SEQ ID NO: 35
KRNYILGLAIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEEASKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG
(exemplary S. aureus dCas9)
SEQ ID NO: 36
MKRNYILGLAIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEEASKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

Similar mutations can also apply to any other naturally-occurring Cas9 (e.g., Cas9 from other species) or engineered Cas9 molecules. In certain embodiments, the dCas9 molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof. In certain embodiments, the present disclosure provides an AAV vector comprising a nucleotide encoding a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof.

In one embodiment, as used herein, “iCas9” and “dCas9” both refer to a Cas9 protein that has the amino acid substitutions D10A and H840A and has its nuclease activity inactivated. In certain embodiments, the Cas9 protein comprises dCas9.

### F. Cas9 Fusion Protein

The CRISPR/Cas9-based system may include a fusion protein. The fusion protein may comprise three heterologous polypeptide domains, wherein the first polypeptide domain comprises, consists of, or consists essentially of a dead Clustered Regularly Interspaced Short Palindromic Repeats associated (dCas) protein, the second polypeptide domain comprises, consists of, or consists essentially of a Kruppel-associated box (KRAB), and the polypeptide domain has an activity selected from the group consisting of transcription activation activity, transcription repression activity, transcription release factor activity, histone modification activity, nuclease activity, nucleic acid association activity, methylase activity, and demethylase activity.

### (1) Transcription Activation Activity

The third polypeptide domain may have transcription activation activity, i.e., a transactivation domain. For example, gene expression of endogenous mammalian genes, such as human genes, may be achieved by targeting a fusion protein of iCas9 and a transactivation domain to mammalian promoters via combinations of gRNAs. The transactivation domain may include a VP 16 protein, multiple VP 16 proteins, such as a VP48 domain or VP64 domain, or p65 domain of NF kappa B transcription activator activity. For example, the fusion protein may be iCas9-VP64.

### (2) Transcription Repression Activity

The third polypeptide domain may have transcription repression activity. The second polypeptide domain may have a Kruppel associated box activity, such as a KRAB domain, ERF repressor domain activity, Mxi1 repressor domain activity, SID4X repressor domain activity, Mad-SID repressor domain activity or TATA box binding protein activity. For example, the fusion protein may be dCas9-KRAB.

### (3) Transcription Release Factor Activity

The third polypeptide domain may have transcription release factor activity. The second polypeptide domain may have eukaryotic release factor 1 (ERF1) activity or eukaryotic release factor 3 (ERF3) activity.

### (4) Histone Modification Activity

The third polypeptide domain may have histone modification activity. The second polypeptide domain may have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300.

### (5) Nuclease Activity

The third polypeptide domain may have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases are deoxyribonuclease and ribonuclease.

### (6) Nucleic Acid Association Activity

The third polypeptide domain may have nucleic acid association activity or nucleic acid binding protein-DNA-binding domain (DBD) is an independently folded protein domain that contains at least one motif that recognizes double- or single-stranded DNA. A DBD can recognize a specific DNA sequence (a recognition sequence) or have a general affinity to DNA. nucleic acid association region selected from the group consisting of helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

### (7) Methylase Activity

The third polypeptide domain may have methylase activity, which involves transferring a methyl group to DNA, RNA, protein, small molecule, cytosine or adenine. The second polypeptide domain may include a DNA methyltransferase.

### (8) Demethylase Activity

The third polypeptide domain may have demethylase activity. The second polypeptide domain may include an enzyme that remove methyl (CH3-) groups from nucleic acids, proteins (in particular histones), and other molecules. Alternatively, the second polypeptide may covert the methyl group to hydroxymethylcytosine in a mechanism for demethylating DNA. The second polypeptide may catalyze this reaction. For example, the second polypeptide that catalyzes this reaction may be Tetl.

In one aspect, the CRISPR/Cas9-based system may include a dCas molecule and a modulator of gene expression, or a nucleic acid encoding a dCas molecule and a modulator of gene expression. In one embodiment, the dCas molecule and the modulator of gene expression are linked covalently. In one embodiment, the modulator of gene expression is covalently fused to the dCas molecule directly. In one embodiment, the modulator of gene expression is covalently fused to the dCas molecule indirectly, e.g., via a non-modulator or linker, or via a second modulator. In one embodiment, the modulator of gene expression is at the N-terminus and/or C-terminus of the dCas molecule. In one embodiment, the dCas molecule and the modulator of gene expression are linked non-covalently. In one embodiment, the dCas molecule is fused to a first tag, e.g., a first peptide tag. In one embodiment, the modulator of gene expression is fused to a second tag, e.g., a second peptide tag. In one embodiment, the first and second tag, e.g., the first peptide tag and the second peptide tag, non-covalently interact with each other, thereby brining the dCas molecule and the modulator of gene expression into close proximity.

In one embodiment, the CRISPR/Cas9-based system includes a fusion molecule or a nucleic acid encoding a fusion molecule. In one embodiment, the fusion molecule comprises a sequence comprising a dCas molecule fused to a modulator of gene expression. In one embodiment, the dCas molecule comprises a Streptococcus pyogenes dCas9 molecule, a Staphylococcus aureus dCas9 molecule, a Campylobacter jejuni dCas9 molecule, a Corynebacterium diphtheria dCas9 molecule, a Eubacterium ventriosum dCas9 molecule, a Streptococcus pasteurianuis dCas9 molecule, a Lactobacillus farciminis dCas9 molecule, a Sphaerochaeta globus dCas9 molecule, an Azospirillum (strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (strain DSM 16511) dCas9 molecule, a Campylobacter lari (strain CF89-12) dCas9 molecule, a Streptococcus thermophilus (strain LMD-9) dCas9 molecule, or fragment thereof. In one embodiment, the modulator of gene expression is chosen from a repressor of gene expression, an activator of gene expression, or a modulator of epigenetic modification.

Different modulators of gene expression are known in the art, see, e.g., Thakore et al., Nat Methods. 2016; 13:127-37, incorporated by reference herein in its entirety.

### (1) Repressor of Gene Expression

The repressor may be any known repressor of gene expression, for example, a repressor chosen from Krüppel associated box (KRAB) domain, mSin3 interaction domain (SID), MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, or fragment or fusion thereof.

### Krüppel Associated Box (KRAB)

The KRAB domain is a type of transcriptional repression domains present in the N-terminal part of many zinc finger protein-based transcription factors. The KRAB domain functions as a transcriptional repressor when tethered to a target DNA by a DNA-binding domain. The KRAB domain is enriched in charged amino acids and can be divided into sub-domains A and B. The KRAB A and B sub-domains can be separated by variable spacer segments and many KRAB proteins contain only the A sub-domain. A sequence of 45 amino acids in the KRAB A sub-domain has been shown to be important for transcriptional repression. The B sub-domain does not repress transcription by itself but does potentiate the repression exerted by the KRAB A sub-domain. The KRAB domain recruits corepressors KAP1 (KRAB-associated protein-1, also known as transcription intermediary factor 1 beta, KRAB-A interacting protein and tripartite motif protein 28) and heterochromatin protein 1 (HP1), as well as other chromatin modulating proteins, leading to transcriptional repression through heterochromatin formation. In one embodiment, the methods and compositions disclosed herein include a fusion molecule comprising a dCas9 molecule fused to a KRAB domain or fragment thereof. In one embodiment, the KRAB domain or fragment thereof is fused to the N-terminus of the dCas9 molecule. In one embodiment, the KRAB domain or fragment thereof is fused to the C-terminus of the dCas9 molecule. In one embodiment, the KRAB domain or fragment thereof is fused to both the N-terminus and the C-terminus of the dCas9 molecule. In one embodiment, the fusion molecule comprises a KRAB domain comprising the sequence of SEQ ID NO: 34, a sequence substantially identical (e.g., at least 80%, 85%, 90%, 92%, 95%, 97%, 98%, 99% or higher identical) to SEQ ID NO: 34, or a sequence having one, two, three, four, five or more changes, e.g., amino acid substitutions, insertions, or deletions, relative to SEQ ID NO: 34, or any fragment thereof.

(exemplary KRAB)
SEQ ID NO: 34
DAKSLTAWSRTLVTFKDVFVDFTREEWKLLDTAQQILYRNVMLENYKNLV
SLGYQLTKPDVILRLEKGEEPWLVEREIHQETHPDSETAFEIKSSVPKKK
RKV

mSin3 Interaction Domain (SID)

The mSin3 interaction domain (SID) is an interaction domain that is present on several transcription repressor proteins. It interacts with the paired amphipathic alpha-helix 2 (PAH2) domain of mSin3, a transcriptional repressor domain that is attached to transcription repressor proteins such as the mSin3A corepressor. In one embodiment, the methods and compositions disclosed herein include a fusion molecule comprising a dCas9 molecule fused to an mSin3 interaction domain or fragment thereof. In one embodiment, the methods and compositions disclosed herein include a fusion molecule comprising a dCas9 molecule fused to four concatenated mSin3 interaction domains (SID4X). In one embodiment, the four concatenated mSin3 interaction domains (SID4X) are fused to the C-terminus of the dCas9 molecule.

### MAX-Interacting Protein 1 (MXI1)

Mxi1 is a repressor of MYC. Mxi1 antagonizes MYC transcriptional activity possibly by competing for binding to MYC-associated factor X (MAX), which binds to MYC and is required for MYC to function. In one embodiment, the methods and compositions disclosed herein include a fusion molecule comprising a dCas9 molecule fused to Mxi1 or fragment thereof. In one embodiment, Mxi1 is fused to the C-terminus of the dCas9 molecule.

### (2) Activator of Gene Expression

The activator may be any known activator of gene expression, for example, a VP16 activation domain, a VP64 activation domain, a p65 activation domain, an Epstein-Barr virus R transactivator Rta molecule, or fragment thereof. Activations that can be used with a dCas9 molecule are known in the art. See, e.g., Chavez et al., Nat Methods. (2016) 13:563-67, incorporated by reference herein in its entirety.

### VP16, VP64, VP160

VP16 is a viral protein sequence of 16 amino acids that recruits transcriptional activators to promoters and enhancers. VP64 is a transcription activator comprising four copies of VP16, e.g., a molecule comprising four tandem copies of VP16 connected by Gly-Ser linkers. VP160 is a transcription activator comprising 10 copies of VP16. In one embodiment, the methods and compositions disclosed herein include a fusion molecule comprising a dCas9 molecule fused to 1, 2, 3, 4, 5, 6, 7, 8, 9, 10, or more copies of VP16. In one embodiment, the methods and compositions disclosed herein include a fusion molecule comprising a dCas9 molecule fused to VP64. In one embodiment, the methods and compositions disclosed herein include a fusion molecule comprising a dCas9 molecule fused to VP160. In one embodiment, VP64 is fused to the C-terminus, the N-terminus, or both the N-terminus and the C-terminus of the dCas9 molecule.

p65 Activation Domain (p65AD)

p65AD is the principal transactivation domain of the 65 kDa polypeptide of the nuclear form of the NF-κB transcription factor. An exemplary sequence of human transcription factor p65 is available at the Uniprot database under accession number Q04206. In one embodiment, the methods and compositions disclosed herein include a fusion molecule comprising a dCas9 molecule fused to p65 or fragment thereof, e.g., p65AD.

### Epstein-Barr Virus (EBV) R Transactivator (Rta)

Rta, an immediate-early protein of EBV, is a transcriptional activator that induces lytic gene expression and triggers virus reactivation. In one embodiment, the methods and compositions disclosed herein include a fusion molecule comprising a dCas9 molecule fused to Rta or fragment thereof.

VP64, p65, Rta Fusions

In one embodiment, the methods and compositions disclosed herein include a fusion molecule comprising a dCas9 molecule fused to VP64, p65, Rta, or any combination thereof. The tripartite activator VP64-p65-Rta (also known as VPR), in which the three transcription activation domains are fused using short amino acid linkers, can effectively up-regulate target gene expression when fused to a dCas9 molecule. In one embodiment, the methods and compositions disclosed herein include a fusion molecule comprising a dCas9 molecule fused to VPR.

### Synergistic Activation Mediators (SAM)

In one embodiment, the methods and compositions disclosed herein include a CRISPR-Cas system that comprises three components: (1) a dCas9-VP64 fusion, (2) a gRNA incorporating two MS2 RNA aptamers at the tetraloop and stem-loop, and (3) the MS2-P65-HSF1 activation helper protein. This system, named Synergistic Activation Mediators (SAM), brings together three activation domains-VP64, P65 and HSF1 and has been described in Konermann et al., Nature. 2015; 517:583-8, incorporated by reference herein in its entirety.

### Ldb1 Self-Association Domain

In one embodiment, the methods and compositions disclosed herein include a fusion molecule comprising a dCas9 molecule fused to Ldb1 self-association domain. Ldb1 self-association domain recruits enhancer-associated endogenous Ldb1.

### (3) Modulator of Epigenetic Modification

In one embodiment, the methods and compositions disclosed herein include a fusion molecule comprising a dCas9 molecule fused to a modular of epigenetic modification. In one embodiment, the fusion molecule modulates target gene expression via epigenetic modification, e.g., via histone acetylation or methylation, or DNA methylation, at a regulatory element of target gene, e.g., a promoter or enhancer. The modulator may be any known modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., LSD1), a DNA methyltransferase (e.g., DNMT3a or DNMT3a-DNMT3L), a DNA demethylase (e.g., TET1 catalytic domain or TDG), or fragment thereof.

In one embodiment, the methods and compositions disclosed herein include a fusion molecule comprising a dCas9 molecule fused to Lys-specific histone demethylase 1 (LSD1) or fragment thereof. In one embodiment, the methods and compositions disclosed herein include a fusion molecule comprising a dCas9 molecule fused to acetyltransferase p300 or fragment thereof, e.g., the catalytic core of p300. In one embodiment, the methods and compositions disclosed herein include a fusion molecule comprising a dCas9 molecule fused to CREB-binding protein (CBP) protein or fragment thereof. In one embodiment, the methods and compositions disclosed herein include a fusion molecule comprising a dCas9 molecule fused to Ten-eleven translocation methylcytosine dioxygenase 1 (TET1) or fragment thereof. In one embodiment, the methods and compositions disclosed herein include a fusion molecule comprising a dCas9 molecule fused to thymine DNA glycosylase (TDG) or fragment thereof. In one embodiment, the methods and compositions disclosed herein include a fusion molecule comprising a dCas9 molecule fused to SUV39H1 or fragment thereof. In one embodiment, the methods and compositions disclosed herein include a fusion molecule comprising a dCas9 molecule fused to G9a (EHMT2) or fragment thereof. In one embodiment, the methods and compositions disclosed herein include a fusion molecule comprising a dCas9 molecule fused to DNMT3a or fragment thereof. In one embodiment, the methods and compositions disclosed herein include a fusion molecule comprising a dCas9 molecule fused to DNMT3a-DNMT3L or fragment thereof.

In one embodiment, the Cas9 fusion protein also comprises a nuclear localization sequence (NLS), e.g., a NLS fused to the N-terminus and/or C-terminus of Cas9. Nuclear localization sequences are known in the art. In one embodiment, the NLS comprises the amino acid sequence of SEQ ID NO: 37 or 38, a sequence substantially identical (e.g., at least 80%, 85%, 90%, 92%, 95%, 97%, 98%, 99% or higher identical) to SEQ ID NO: 37 or 38, or a sequence having one, two, three, four, five or more changes, e.g., amino acid substitutions, insertions, or deletions, relative to SEQ ID NO: 37 or 38, or any fragment thereof.

(exemplary nuclear localization sequence)
SEQ ID NO: 37
APKKKRKVGIHGVPAA
(exemplary nuclear localization sequence)
SEQ ID NO: 38
KRPAATKKAGQAKKKK

In one embodiment, the fusion molecule is a NLS-dSaCas9-NLS-KRAB fusion molecule comprising from the N-terminus to the C-terminus: a first NLS, an S. aureus dCas9 molecule, a second NLS, and a KRAB, fused directly or indirectly (e.g., via a linker). In one embodiment, the fusion molecule is a HA-NLS-dSaCas9-NLS-KRAB fusion molecule comprising from the N-terminus to the C-terminus: a HA tag, a first NLS, an S. aureus dCas9 molecule, a second NLS, and a KRAB, fused directly or indirectly (e.g., via a linker). In one embodiment, the fusion molecule is encoded by a nucleic acid comprising the sequence of SEQ ID NO: 23, a sequence substantially identical (e.g., at least 80%, 85%, 90%, 92%, 95%, 97%, 98%, 99% or higher identical) to SEQ ID NO: 23, or a sequence having one, two, three, four, five or more changes, e.g., substitutions, insertions, or deletions, relative to SEQ ID NO: 23, or any fragment thereof. In one embodiment, the fusion molecule comprises the fusion molecule comprises the amino acid sequence of SEQ ID NO: 39, 40, or 41, a sequence substantially identical (e.g., at least 80%, 85%, 90%, 92%, 95%, 97%, 98%, 99% or higher identical) to SEQ ID NO: 39, 40, or 41, or a sequence having one, two, three, four, five or more changes, e.g., amino acid substitutions, insertions, or deletions, relative to SEQ ID NO: 39, 40, or 41, or any fragment thereof.

(exemplary HA-NLS-dSaCas9-NLS-KRAB sequence)
SEQ ID NO: 39
MYPYDVPDYAAPKKKRKVGIHGVPAAKRNYILGLAIGITSVGYGIIDYET
RDVIDAGVRLFKEANVENNEGRRSKRGARRLKRRRRHRIQRVKKLLFDYN
LLTDHSELSGINPYEARVKGLSQKLSEEEFSAALLHLAKRRGVHNVNEVE
EDTGNELSTKEQISRNSKALEEKYVAELQLERLKKDGEVRGSINRFKTSD
YVKEAKQLLKVQKAYHQLDQSFIDTYIDLLETRRTYYEGPGEGSPFGWKD
IKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTN
LKVYHDIKDITARKEIIENAELLDQIAKILTIYQSSEDIQEELTNLNSEL
TQEEIEQISNLKGYTGTHNLSLKAINLILDELWHTNDNQIAIFNRLKLVP
KKVDLSQQKEIPTTLVDDFILSPVVKRSFIQSIKVINAIIKKYGLPNDII
IELAREKNSKDAQKMINEMQKRNRQTNERIEEIIRTTGKENAKYLIEKIK
LHDMQEGKCLYSLEAIPLEDLLNNPFNYEVDHIIPRSVSFDNSFNNKVLV
KQEEASKKGNRTPFQYLSSSDSKISYETFKKHILNLAKGKGRISKTKKEY
LLEERDINRFSVQKDFINRNLVDTRYATRGLMNLLRSYFRVNNLDVKVKS
INGGFTSFLRRKWKFKKERNKGYKHHAEDALIIANADFIFKEWKKLDKAK
KVMENQMFEEKQAESMPEIETEQEYKEIFITPHQIKHIKDFKDYKYSHRV
DKKPNRELINDTLYSTRKDDKGNTLIVNNLNGLYDKDNDKLKKLINKSPE
KLLMYHHDPQTYQKLKLIMEQYGDEKNPLYKYYEETGNYLTKYSKKDNGP
VIKKIKYYGNKLNAHLDITDDYPNSRNKVVKLSLKPYRFDVYLDNGVYKF
VTVKNLDVIKKENYYEVNSKCYEEAKKLKKISNQAEFIASFYNNDLIKIN
GELYRVIGVNNDLLNRIEVNMIDITYREYLENMNDKRPPRIIKTIASKTQ
SIKKYSTDILGNLYEVKSKKHPQIIKKGKRPAATKKAGQAKKKKGSDAKS
LTAWSRTLVTFKDVFVDFTREEWKLLDTAQQILYRNVMLENYKNLVSLGY
QLTKPDVILRLEKGEEPWLVEREIHQETHPDSETAFEIKSSVPKKKRKV
(exemplary HA-NLS-dSaCas9-NLS-KRAB sequence)
SEQ ID NO: 40
YPYDVPDYAAPKKKRKVGIHGVPAAKRNYILGLAIGITSVGYGIIDYETR
DVIDAGVRLFKEANVENNEGRRSKRGARRLKRRRRHRIQRVKKLLFDYNL
LTDHSELSGINPYEARVKGLSQKLSEEEFSAALLHLAKRRGVHNVNEVEE
DTGNELSTKEQISRNSKALEEKYVAELQLERLKKDGEVRGSINRFKTSDY
VKEAKQLLKVQKAYHQLDQSFIDTYIDLLETRRTYYEGPGEGSPFGWKDI
KEWYEMLMGHCTYFPEELRSVKYAYNADLYNALNDLNNLVITRDENEKLE
YYEKFQIIENVFKQKKKPTLKQIAKEILVNEEDIKGYRVTSTGKPEFTNL
KVYHDIKDITARKEIIENAELLDQIAKILTIYQSSEDIQEELTNLNSELT
QEEIEQISNLKGYTGTHNLSLKAINLILDELWHTNDNQIAIFNRLKLVPK
KVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKL
HDMQEGKCLYSLEAIPLEDLLNNPFNYEVDHIIPRSVSFDNSFNNKVLVK
QEEASKKGNRTPFQYLSSSDSKISYETFKKHILNLAKGKGRISKTKKEYL
LEERDINRFSVQKDFINRNLVDTRYATRGLMNLLRSYFRVNNLDVKVKSI
NGGFTSFLRRKWKFKKERNKGYKHHAEDALIIANADFIFKEWKKLDKAKK
VMENQMFEEKQAESMPEIETEQEYKEIFITPHQIKHIKDFKDYKYSHRVD
KKPNRELINDTLYSTRKDDKGNTLIVNNLNGLYDKDNDKLKKLINKSPEK
LLMYHHDPQTYQKLKLIMEQYGDEKNPLYKYYEETGNYLTKYSKKDNGPV
IKKIKYYGNKLNAHLDITDDYPNSRNKVVKLSLKPYRFDVYLDNGVYKFV
TVKNLDVIKKENYYEVNSKCYEEAKKLKKISNQAEFIASFYNNDLIKING
ELYRVIGVNNDLLNRIEVNMIDITYREYLENMNDKRPPRIIKTIASKTQS
IKKYSTDILGNLYEVKSKKHPQIIKKGKRPAATKKAGQAKKKKGSDAKSL
TAWSRTLVTFKDVFVDFTREEWKLLDTAQQILYRNVMLENYKNLVSLGYQ
LTKPDVILRLEKGEEPWLVEREIHQETHPDSETAFEIKSSVPKKKRKV
(exemplary NLS-dSaCas9-NLS-KRAB)
SEQ ID NO: 41
APKKKRKVGIHGVPAAKRNYILGLAIGITSVGYGIIDYETRDVIDAGVRL
FKEANVENNEGRRSKRGARRLKRRRRHRIQRVKKLLFDYNLLTDHSELSG
INPYEARVKGLSQKLSEEEFSAALLHLAKRRGVHNVNEVEEDTGNELSTK
EQISRNSKALEEKYVAELQLERLKKDGEVRGSINRFKTSDYVKEAKQLLK
VQKAYHQLDQSFIDTYIDLLETRRTYYEGPGEGSPFGWKDIKEWYEMLMG
HCTYFPEELRSVKYAYNADLYNALNDLNNLVITRDENEKLEYYEKFQIIE
NVFKQKKKPTLKQIAKEILVNEEDIKGYRVTSTGKPEFTNLKVYHDIKDI
TARKEIIENAELLDQIAKILTIYQSSEDIQEELTNLNSELTQEEIEQISN
LKGYTGTHNLSLKAINLILDELWHTNDNQIAIFNRLKLVPKKVDLSQQKE
IPTTLVDDFILSPVVKRSFIQSIKVINAIIKKYGLPNDIIIELAREKNSK
DAQKMINEMQKRNRQTNERIEEIIRTTGKENAKYLIEKIKLHDMQEGKCL
YSLEAIPLEDLLNNPFNYEVDHIIPRSVSFDNSFNNKVLVKQEEASKKGN
RTPFQYLSSSDSKISYETFKKHILNLAKGKGRISKTKKEYLLEERDINRF
SVQKDFINRNLVDTRYATRGLMNLLRSYFRVNNLDVKVKSINGGFTSFLR
RKWKFKKERNKGYKHHAEDALIIANADFIFKEWKKLDKAKKVMENQMFEE
KQAESMPEIETEQEYKEIFITPHQIKHIKDFKDYKYSHRVDKKPNRELIN
DTLYSTRKDDKGNTLIVNNLNGLYDKDNDKLKKLINKSPEKLLMYHHDPQ
TYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIK
KENYYEVNSKCYEEAKKLKKISNQAEFIASFYNNDLIKINGELYRVIGVN
NDLLNRIEVNMIDITYREYLENMNDKRPPRIIKTIASKTQSIKKYSTDIL
GNLYEVKSKKHPQIIKKGKRPAATKKAGQAKKKKGSDAKSLTAWSRTLVT
FKDVFVDFTREEWKLLDTAQQILYRNVMLENYKNLVSLGYQLTKPDVILR
LEKGEEPWLVEREIHQETHPDSETAFEIKSSVPKKKRKV

### G. gRNA

As described above, the CRISPR/Cas9 system utilizes gRNA that provides the targeting of the CRISPR/Cas9-based system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The term “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

The number of gRNA administered to the cell may be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 19 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA administered to the cell may be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

In one embodiment, the gRNA is selected to increase or decrease transcription of a target gene. In one embodiment, the gRNA targets a region upstream of the transcription start site of a target gene, e.g., between 0-1000 bp upstream of the transcription start site of a target gene. In one embodiment, the gRNA targets a region downstream of the transcription start site of a target gene, e.g., between 0-1000 bp downstream of the transcription start site of a target gene. In one embodiment, the gRNA targets a promoter region of a target gene. In one embodiment, the gRNA targets an enhancer region of a target gene.

gRNA can be divided into a target binding region, a Cas9 binding region, and a transcription termination region. The target binding region hybridizes with a target region in a target gene. Methods for designing such target binding regions are known in the art, see, e.g., Doench et al., Nat Biotechnol. (2014) 32:1262-7; and Doench et al., Nat Biotechnol. (2016) 34:184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length. In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

In one embodiment, the target binding region is engineered to improve stability or extend half-life, e.g., by incorporating a non-natural nucleotide or a modified nucleotide in the target binding region, by removing or modifying an RNA destabilizing sequence element, by adding an RNA stabilizing sequence element, or by increasing the stability of the Cas9/gRNA complex. In one embodiment, the target binding region is engineered to enhance its transcription. In one embodiment, the target binding region is engineered to reduce secondary structure formation.

In one embodiment, the Cas9 binding region of gRNA is modified to enhance the transcription of the gRNA. In one embodiment, the Cas9 binding region of gRNA is modified to improve stability or assembly of the Cas9/gRNA complex.

### H. Gene Therapy Construct

Another aspect of the present disclosure provides a gene therapy construct comprising, consisting of, or consisting essentially of a fusion protein comprising three heterologous polypeptide domains, wherein the first polypeptide domain comprises, consists of, or consists essentially of a dead Clustered Regularly Interspaced Short Palindromic Repeats associated (dCas) protein, the second polypeptide domain comprises, consists of, or consists essentially of a Kruppel-associated box (KRAB), and the polypeptide domain has an activity selected from the group consisting of transcription activation activity, transcription repression activity, transcription release factor activity, histone modification activity, nuclease activity, nucleic acid association activity, methylase activity, and demethylase activity.

In one aspect, the present disclosure provides a nucleic acid encoding a fusion protein comprising a dCas9 molecule fused to a modulator of gene expression. In one embodiment, the nucleic acid contains a promoter operably linked to a polynucleotide encoding the fusion protein. In one embodiment, the promoter is constitutive. In one embodiment, the promoter is inducible. In one embodiment, the promoter is tissue specific. In one embodiment, the promoter is specific for liver expression. In one embodiment, the promoter for the polynucleotide encoding the fusion protein is selected to express an amount of the fusion protein that is proportional to the amount of gRNA, or amount of gRNA expression.

In another aspect, the present disclosure provides a nucleic acid encoding gRNA. In one embodiment, the nucleic acid contains a promoter operably linked to a polynucleotide encoding the gRNA. In one embodiment, the promoter is constitutive. In one embodiment, the promoter is inducible. In one embodiment, the promoter is tissue specific. In one embodiment, the promoter is specific for liver expression. In one embodiment, the promoter for the polynucleotide encoding the gRNA is selected to express an amount of the gRNA that is proportional to the amount of the fusion protein, or amount of fusion protein expression.

In some embodiments, the gene therapy construct comprises a vector system. In certain embodiments, the vector system comprises an AAV vector system.

In another embodiment, the gene therapy construct further comprises a first and second AAV inverted terminal repeat (ITR) sequence flanking the fusion protein.

In one embodiment, the vector system is a single viral vector system comprising a viral vector. In one embodiment, the vector is an adeno-associated virus (AAV) vector. In one embodiment, the adeno-associated virus is selected from the serotype 2, the serotype 5, the serotype 7, the serotype 8, and the serotype 9. In one embodiment, the vector comprises a first nucleic acid molecule that encodes a fusion molecule comprising a dCas9 molecule fused to a modulator that regulates the expression of a gene, and a second nucleic acid molecule that encodes a gRNA that targets the fusion molecule to the gene.

In one embodiment, the vector system comprises two or more viral vectors. In one embodiment, the vector system is a dual viral vector system comprising a first viral vector and a second viral vector. In one embodiment, the first and second vectors are adeno-associated virus (AAV) vectors. In one embodiment, the adeno-associated virus (AAV) vectors are the same or different AAV serotypes. In one embodiment, the adeno-associated virus is selected from the serotype 2, the serotype 5, the serotype 7, the serotype 8, and the serotype 9. In one embodiment, the first vector comprises a first nucleic acid molecule that encodes a fusion molecule comprising a dCas9 molecule fused to a modulator that regulates the expression of a gene; and the second vector comprises a second nucleic acid molecule that encodes a gRNA that targets the fusion molecule to the gene.

Different AAV capsids may be used in the compositions and methods described herein. For example, suitable AAV includes, but is not limited to, AAV8 (see, e.g., U.S. Pat. Nos. 7,790,449 and 7,282,199, incorporated by reference herein in their entirety), AAV9 (see, e.g., U.S. Pat. No. 7,906,111 and US 2011/0236353, incorporated by reference herein in their entirety), hu.37 (see, e.g., U.S. Pat. No. 7,906,111 and US 2011/0236353, incorporated by reference herein in their entirety), AAV1, AAV2, AAV3, AAV4, AAV5, AAV6, AAV6.2, AAV7, and AAV8 (see, e.g., U.S. Pat. Nos. 7,790,449 and 7,282,199, incorporated by reference herein in their entirety). The sequences of additional suitable AAV vectors and methods for generating them are disclosed in WO 2003/042397, WO 2005/033321, WO 2006/110689, U.S. Pat. Nos. 7,790,449, 7,282,199, and 7,588,772, incorporated by reference herein in their entirety. Still other AAV may be selected, optionally taking into consideration tissue preferences of the selected AAV capsid. A recombinant AAV vector (AAV viral particle) may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., McCarty et al, Gene Ther. 2001, 8:1248-54, incorporated by reference herein in its entirety. Self-complementary AAVs are described in, e.g., U.S. Pat. Nos. 6,596,535; 7,125,717, and 7,456,683, incorporated by reference herein in their entirety.

A single-stranded AAV viral vector may be used. Methods for generating and isolating AAV viral vectors suitable for delivery to a subject are known in the art. See, e.g., U.S. Pat. Nos. 7,790,449; 7,282,199; WO 2003/042397; WO 2005/033321; WO 2006/110689; and U.S. Pat. No. 7,588,772. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., Hum Gene Ther. 2009; 20:922-9, incorporated by reference herein in its entirety. Methods of making and using these and other AAV production systems are also described in the following U.S. patents, incorporated by reference herein in their entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus vectors. Suitably, where one of these other vectors is generated, it is produced as a replication-defective viral vector. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production.

In another embodiment, a non-viral delivery system may be used. For example, a composition disclosed herein comprising a nucleic acid may be formulated with nanoparticles, micelles, liposomes, cationic lipids, poly-glycans, polymers, lipids and/or cholesterols. See, e.g., Su et al., Mol. Pharmaceutics, 2011, 8, 774-787; WO 2013/182683, WO 2010/053572, and WO 2012/170930, incorporated by reference herein in their entirety.

Another aspect of the present disclosure provides a pharmaceutical composition comprising the gene therapy construct as described herein in a biocompatible pharmaceutical carrier.

In another aspect, the present disclosure provides a modified programmable RNA-guided dCas9-based repressor for efficient packaging in AAV and in vivo gene regulation. This gene delivery system can be customized to target any endogenous gene by designing a new guide RNA molecule, enabling patent and stable gene repression in animal models and therapeutic use.

In some embodiments, the Cas protein comprises Cas9.

In some embodiments, the gene therapy construct is designed for the targeted reduction of the PCSK9 gene.

### I. Gene Therapy Target

The invention disclosed herein can be used to modulate the expression of a gene of interest. In one embodiment, the expression of the gene is down-regulated. In one embodiment, the expression of the gene is up-regulated. In one embodiment, the temporal pattern of the expression of the gene is modulated. In one embodiment, the spatial pattern of the expression of the gene is modulated. Exemplary genes, tissues expressing these genes, and relevant disease indications are disclosed in Tables 2 and 3. Table 2 provides genes, the expression of which can be down-regulated to treat diseases shown alongside the genes. Table 3 provides genes, the expression of which can be up-regulated to treat diseases shown alongside the genes.

TABLE 2
Exemplary genes for expression modulation (e.g.,
repression) and Exemplary Diseases and Tissues

| Gene | Disease | Tissue |
|---|---|---|
| proprotein convertase | Hypercholesteremia | Liver |
| subtilisin/kexin type | | |
| 9 (PCSK9) | | |
| activin receptor type- | muscle weakness | Muscle |
| 2B (ACVR2B) | | |
| huntingtin gene (HTT) | Huntington's disease | Brain |
| superoxide dismutase 1 | Amyotrophic lateral sclerosis | Brain |
| (SOD1) | | |
| transthyretin (TTR) | Hereditary ATTR amyloidosis | Liver |
| antithrombin | Hemophilia | Liver |
| complement component C5 | Complement-mediated disease | Liver |
| aminolevulinic acid | Hepatic porphyria | Liver |
| synthase 1 | | |
| glycolate oxidase | Primary hyperoxaluria type 1 | Liver |
| transmembrane protease, | Beta thalassemia | Liver |
| serine 6 (Tmprss6) | | |
| alpha-antitrypsin (AAT) | Alpha-1 antitrypsin (AAT) | Liver |
| | deficiency | |
| vascular endothelial | Age-related macular | Retina |
| growth factor (VEGF) | degeneration | |
| C9orf72 | Familial frontotemporal | Brain |
| | dementia (FTD) and | |
| | amyotrophic lateral | |
| | sclerosis (ALS) | |
| KRAS | Cancer | tumor |
| human epidermal growth | Cancer | tumor |
| factor receptor 2 (HER2) | | |
| Beta catenin | Cancer | tumor |
| angiopoietin-like 3 | Hyperlipidemia | Liver |
| (ANGPTL3) | | |
| apolipoprotein C-III | Hyperlipidemia | Liver |
| (apoCIII) | | |
| PD-L1 | Chronic liver infection | Liver |
| HBV, HCV, HDV viral | Hepatitis | Liver |
| genomes | | |
| vascular endothelial | Age-related macular | Retina |
| growth factor receptor | degeneration | |
| 1 (VEGFR1) | | |
| RTP801 | Age-related macular | Retina |
| | degeneration | |
| beta-2 adrenergic | Glaucoma, Ocular | Retina |
| receptor (ADRB2) | hypertension | |
| Caspase 2 | Glaucoma, Ocular | Retina |
| | hypertension | |
| IKKbeta | Glaucoma | Retina |
| apolipoprotein A | Cardiovascular disease | Liver |
| factor 12 | Hereditary angioedema | Liver |
| prekallikrein | Hereditary angioedema | Liver |
| apolipoprotein B-100 | Hypercholesteremia | Liver |
| glucagon receptor | Diabetes | Liver |
| microRNA-103/107 | Nonalcoholic steatohepatitis | Liver |
| | (NASH) in patients with | |
| | type 2 diabetes | |
| Diacylglycerol O- | Nonalcoholic steatohepatitis | Liver |
| Acyltransferase 2 | (NASH) in patients with | |
| (DGAT2) | type 2 diabetes | |
| Ube3a-ATS | Angelman Syndrome | Brain |
| TNFR | Autoimmmune disease | Various- |
| | | cartilage |
| FRG1 | Facioscapulohumeral muscular | Muscle |
| | dystrophy | |
| BCR-ABL | Chronic myelogenous leukemia | Blood |
| | | tumor |
| TEL-AML1 | Acute lymphoblastic leukemia | Blood |
| | | tumor |
| PTEN | Cancer | Tumor |
| Other tumor suppressors | Cancer | Tumor |
| Mendelian disorders | Various | Various |
| Triggering receptor | Neurodegenerative disease, | CNS |
| expressed on myeloid | e.g., Alzheimer's disease, | |
| cells 2 (TREM-2) | amyotrophic lateral | |
| | sclerosis, and Parkinson's | |
| | disease | |
| APOE4 | Alzheimer's disease | CNS |
| CD33 | Alzheimer's disease | CNS |
| Other disease risk genes | Various | Various |

TABLE 3
Exemplary genes for expression modulation (e.g.,
activation) and Exemplary Diseases and Tissues

| Gene | Disease | Tissue |
|---|---|---|
| aromatic L-amino acid | Parkinson's disease | Brain |
| decarboxylase (AADC) | | |
| triggering receptor | Alzheimer's Disease | Brain |
| expressed on myeloid cells | | |
| 2 (TREM2) | | |
| vascular endothelial growth | Tissue regeneration | Various- |
| factor (VEGF) | | muscle |
| brain-derived neurotrophic | Neurological conditions | Brain |
| factor (BDNF) | | |
| platelet-derived growth | Tissue regeneration | Various- |
| factor (PDGF) | | muscle |
| utrophin | Muscular dystrophy | Skeletal and |
| | | cardiac muscle |
| frataxin | Friedreich's ataxia | Brain |
| sodium voltage-gated | Dravet Syndrome | Brain |
| channel alpha subunit 1 | | |
| (SCN1A) | | |
| pigment epithelium-derived | Wet AMD, cancer | Eye, tumor |
| factor (PEDF) | | |
| BCL2 Associated X (BAX) | Cancer | Tumor |
| mammary serine protease | Cancer | Tumor |
| inhibitor (maspin) | | |
| p53 | Cancer | Tumor |
| cystic fibrosis | Cystic fibrosis | Lung |
| transmembrane | | |
| conductance regulator | | |
| (CFTR) | | |
| fragile X mental retardation | Fragile X | Brain |
| 1 (FMR1) | | |
| methyl-CpG-binding | Rhett syndrome | Brain |
| protein 2 (MECP2) | | |
| ubiquitin-protein ligase | Angelman syndrome | Brain |
| E3A (Ube3a) | | |
| ubiquitin-protein ligase | Prader-Willi syndrome | Brain |
| E3A (Ube3a) | | |
| IL1RA | rheumatoid arthritis | Cartilage |
| HBG1/HBG2 | sickle cell anemia | Blood |
| IL-10 | Collitis, inflammatory | Gut-T |
| | bowel disease | cells |
| IL-2 | Various-graft versus | Various |
| | host disease, | |
| | rheumatoid arthritis, | |
| | lupus, type 1 diabetes | |
| Growth factors (e.g., having | Various | Various |
| a protective or regenerative | | |
| function) | | |

### J. Methods

A variety of different diseases and conditions (e.g., one or more diseases described herein), e.g., diseases and conditions associated with one or more genes described herein, including, e.g., genetic deletions, insertions or mutations, can be treated using the method described herein. The compositions described herein can be delivered to any of the cells, tissues, or organs described herein to treat a disorder or condition associated with a gene described herein. Exemplary genes for expression modulation (e.g., repression or activation), and exemplary diseases and tissues, are described in Tables 2 and 3.

In one aspect, the present disclosure provides a method of suppressing the expression of a gene in a cell in vivo comprising, consisting of, or consisting essentially of administering to a cell a therapeutically effective amount of a gene therapy construct as described herein such that the gene expression is suppressed.

In one aspect, the present disclosure provides a method of suppressing the expression of a gene in vivo in a subject comprising, consisting of, or consisting essentially of administering to the subject a therapeutically effective amount of a gene therapy construct as described herein such that the gene expression is suppressed.

In some embodiments, the method is designed for the targeted reduction of the PCSK9 gene. In some embodiments, the method is designed for the targeted reduction of the expression of the PCSK9 gene.

In one aspect, the present disclosure provides a method of increasing the expression of a gene in a cell in vivo comprising, consisting of, or consisting essentially of administering to a cell a therapeutically effective amount of a gene therapy construct as described herein such that the gene expression is increased.

In one aspect, the present disclosure provides a method of increasing the expression of a gene in vivo in a subject comprising, consisting of, or consisting essentially of administering to the subject a therapeutically effective amount of a gene therapy construct as described herein such that the gene expression is increased.

In one embodiment, the aforementioned methods comprise administering to the cell or subject: a first nucleic acid that encodes a fusion molecule comprising a sequence comprising a dCas9 molecule fused to a modulator of gene expression, and a second nucleic acid that encodes a gRNA which targets the fusion molecule to the gene, in an amount sufficient to modulate expression of the gene. In one embodiment, the first and second nucleic acids are packaged in a same vector or different vectors. In one embodiment the first and second nucleic acids are packaged in a same AAV vector or different AAV vectors. In one embodiment, the first nucleic acid is a DNA. In one embodiment, the first nucleic acid is an mRNA.

In one embodiment, the aforementioned methods comprise administering to the cell or subject: a fusion molecule comprising a sequence comprising a dCas9 molecule fused to a modulator of gene expression, and a nucleic acid that encodes a gRNA which targets the fusion molecule to the gene, in an amount sufficient to modulate expression of the gene. In one embodiment, the nucleic acid is packaged in a viral vector, e.g., an AAV vector.

In one embodiment, the aforementioned methods comprise administering to the cell or subject: a fusion molecule comprising a sequence comprising a dCas9 molecule fused to a modulator of gene expression, and a gRNA which targets the fusion molecule to the gene, in an amount sufficient to modulate expression of the gene.

In one embodiment, the aforementioned methods comprise administering to the cell or subject: a nucleic acid that encodes a fusion molecule comprising a sequence comprising a dCas9 molecule fused to a modulator of gene expression, and a gRNA which targets the fusion molecule to the gene, in an amount sufficient to modulate expression of the gene. In one embodiment, the nucleic acid is packaged in a viral vector, e.g., an AAV vector. In one embodiment, the nucleic acid is a DNA. In one embodiment, the nucleic acid is an mRNA.

Different administration routes may be used for the methods disclosed herein. The compositions disclosed herein can be administered systemically or locally. In some embodiments, the compositions disclosed herein are administered intravenously, subcutaneously, orally, via inhalation, intranasally, intratracheally, intraarterially, intraocularly, or intramuscularly. In some embodiments, the compositions may be delivered in a single administration or multiple administrations. In one embodiment, two or more AAV vectors may be delivered, see, e.g., WO 2011/126808 and WO 2013/049493, incorporated by reference herein in their entirety.

In the case of AAV viral vectors, quantification of the genome copies (“GC”) may be used as the measure of the dose contained in the formulation. Any method known in the art can be used to determine the genome copy (GC) number of the replication-defective virus compositions of the invention.

Production of lentivirus is measured as IU per volume (e.g., mL). IU is infectious unit, or alternatively transduction units (TU); IU and TU can be used interchangeably as a quantitative measure of the titer of a viral vector particle preparation.

Any known RNA delivery method can be used in the methods disclosed herein, including but not limited to, delivering RNA using block copolymers (see, e.g., US 2011/0286957, EP2620161, and WO 2015/017519, incorporated by reference herein in their entirety), and delivering RNA using cationic complexes or liposomal formulations (see, .e.g., Landen et al., Cancer Biol. Ther. (2006) 5 (12); Khoury et al., Arthritis Rheumatol. (2006) 54:1867-77, incorporated by reference herein in their entirety). Local administration to the liver has also been demonstrated by injecting double stranded RNA directly into the circulatory system surrounding the liver using renal vein catheterization, see, e.g., Hamar et al., PNAS (2004) 101:14883-8, incorporated by reference herein in its entirety.

Other methods are disclosed in WO 2013/143555; US 2013/0323001; US 2012/0195917; Soutschek et al., Nature (2004) 432:173-8; Morrissey et al., Hepatol. (2005) 41:1349-56; Uchida et al, (2013) PLOS ONE 8: e56220, incorporated by reference herein in their entirety.

### K. Kits

Another aspect of the present disclosure provides a kit for the suppression of a gene in vivo comprising a gene therapy construct or pharmaceutical composition as described herein and instructions for use.

Yet another aspect of the present disclosure provides all that is described and illustrated herein.

The present invention may be defined in any of the following numbered paragraphs:
1. A method of modulating expression of a gene, in vivo, in a subject comprising administering to, or providing in, the subject:
(a)(i) a fusion molecule comprising a sequence comprising a dCas9 molecule fused to a modulator of gene expression; or (ii) a nucleic acid that encodes a fusion molecule comprising a sequence comprising a dCas9 molecule fused to a modulator of gene expression; and
(b)(i) a gRNA which targets the fusion molecule to the gene; or (ii) a nucleic acid that encodes a gRNA which targets the fusion molecule to the gene,
in an amount sufficient to modulate expression of the gene.
2. The method of paragraph 1, comprising administering to, or provided in, the subject any of: (a)(ii) and (b)(ii), (a)(i) and (b)(i), (a)(i) and (b)(ii), or (a)(ii) and (b)(i).
3. The method of paragraph 1 or 2, comprising administering to, or provided in, the subject:
(a)(ii) a nucleic acid that encodes a fusion molecule comprising a sequence comprising a dCas9 molecule fused to a modulator of gene expression; and
(b)(ii) a nucleic acid that encodes a gRNA which targets the fusion molecule to the gene.
4. The method of any of the preceding paragraphs, wherein the nucleic acid of (a)(ii) comprises DNA.
5. The method of any of the preceding paragraphs, wherein the nucleic acid of (b)(ii) comprises DNA.
6. The method of any of the preceding paragraphs, wherein the nucleic acid of (a)(ii) comprises RNA.
7. The method of any of the preceding paragraphs, wherein the nucleic acid of (b)(ii) comprises RNA.
8. The method of any of the preceding paragraphs, wherein one or both of (a) and (b) are packaged in a viral vector.
9. The method of any of the preceding paragraphs, wherein (a) is packaged in a viral vector.
10. The method of any of the preceding paragraphs, wherein (b) is packaged in a viral vector.
11. The method of any of the preceding paragraphs, wherein (a) and (b) are packaged in the same viral vector.
12. The method of any of paragraphs 8-11, wherein the viral vector comprises an AAV vector.
13. The method of any of paragraphs 8-11, wherein the viral vector comprises a lentiviral vector.
14. The method of any of paragraphs 1-10, wherein (a) is packaged in a first viral vector and (b) is packaged in a second viral vector.
15. The method of paragraph 14, wherein the first viral vector comprises an AAV vector and the second viral vector comprises an AAV vector.
16. The method of any of the preceding paragraphs, wherein the dCas9 molecule comprises a gRNA binding domain of a Cas9 molecule.
17. The method of any of the preceding paragraphs, wherein the dCas9 molecule comprises one, two or all of: a Rec1 domain, a bridge helix domain, or a PAM interacting domain, of a Cas9 molecule.
18. The method of any of the preceding paragraphs, wherein the dCas9 molecule is a mutant of a wild-type Cas9 molecule, e.g., in which the Cas9 nuclease activity is inactivated.
19. The method of any of the preceding paragraphs, wherein the dCas9 molecule comprises a mutation that inactivates a Cas9 nuclease activity, e.g., a mutation in a DNA-cleavage domain of a Cas9 molecule.
20. The method of any of the preceding paragraphs, wherein the dCas9 molecule comprises a mutation that inactivates a Cas9 nuclease activity, e.g., a mutation in a RuvC domain and/or a mutation in a HNH domain.
21. The method of any of the preceding paragraphs, wherein the dCas9 molecule comprises a Staphylococcus aureus dCas9 molecule, a Streptococcus pyogenes dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (e.g., strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (e.g., strain DSM 16511) dCas9 molecule, a Campylobacter lari (e.g., strain CF89-12) dCas9 molecule, or a Streptococcus thermophilus (e.g., strain LMD-9) dCas9 molecule.
22. The method of any of the preceding paragraphs, wherein the dCas9 molecule comprises an S. aureus dCas9 molecule, e.g., comprising an S. aureus dCas9 sequence described herein.
23. The method of any of the preceding paragraphs, wherein the S. aureus dCas9 molecule comprises a mutation at an amino acid position, corresponding to position 10, 580, or both (e.g., D10A, N580A, or both), relative to a wild-type S. aureus dCas9 molecule, numbered according to SEQ ID NO: 25.
24. The method of any of the preceding paragraphs, wherein the S. aureus dCas9 molecule comprises the amino acid sequence of SEQ ID NO: 35 or 36, a sequence substantially identical (e.g., at least 80%, 85%, 90%, 92%, 95%, 97%, 98%, 99% or higher identical) to SEQ ID NO: 35 or 36, or a sequence having one, two, three, four, five or more changes, e.g., amino acid substitutions, insertions, or deletions, relative to SEQ ID NO: 35 or 36, or any fragment thereof.
25. The method of any of paragraphs 1-20, wherein the dCas9 molecule comprises an S. pyogenes dCas9 molecule, e.g., comprising an S. pyogenes dCas9 sequence described herein.
26. The method of any of paragraphs 1-20, the S. pyogenes dCas9 molecule comprises a mutation at an amino acid position, corresponding to position 10, 840, or both (e.g., D10A, H840A, or both), relative to a wild-type S. pyogenes dCas9 molecule, numbered according to SEQ ID NO: 24.
27. The method of any of the preceding paragraphs, wherein the dCas9 molecule is less than 1400, 1300, 1200, 1100, 1000, 900, 800, 700, 600, or 500 amino acids in length.
28. The method of any of the preceding paragraphs, wherein the dCas9 molecule is 500-1300, 600-1200, 700-1100, 800-1000, 500-1200, 500-1000, 500-800, 500-600, 1000-1200, 800-1200, or 600-1200 amino acids in length.
29. The method of any of the preceding paragraphs, wherein the dCas9 molecule has a size that is less than 90%, 80%, 70%, 60%, 50%, 40%, or 30% of the size of a wild-type Cas9 molecule, e.g., a wild-type S. pyogenes Cas9 molecule or a wild-type S. aureus dCas9 molecule.
30. The method of any of the preceding paragraphs, wherein the modulator of gene expression comprises a modulator of gene expression described herein.
31. The method of any of the preceding paragraphs, wherein the modulator of gene expression comprises a repressor of gene expression, e.g., a Krüppel associated box (KRAB) molecule, an mSin3 interaction domain (SID) molecule, four concatenated mSin3 interaction domains (SID4X), MAX-interacting protein 1 (MXI1), or any fragment thereof.
32. The method of any of the preceding paragraphs, wherein the modulator of gene expression comprises a Krüppel associated box (KRAB) molecule comprising the sequence of SEQ ID NO: 34, a sequence substantially identical (e.g., at least 80%, 85%, 90%, 92%, 95%, 97%, 98%, 99% or higher identical) to SEQ ID NO: 34, or a sequence having one, two, three, four, five or more changes, e.g., amino acid substitutions, insertions, or deletions, relative to SEQ ID NO: 34, or any fragment thereof.
33. The method of any of the preceding paragraphs, wherein the modulator of gene expression comprises an activator of gene expression, e.g., a VP16 transcription activation domain, a VP64 transcriptional activation domain, a p65 activation domain, an Epstein-Barr virus R transactivator Rta molecule, a VP64-p65-Rta fusion (VPR), Ldb1 self-association domain, or any fragment thereof.
34. The method of any of the preceding paragraphs, wherein the modulator of gene expression comprises a modulator of epigenetic modification, e.g., a histone acetyltransferase (e.g., p300 catalytic domain), a histone deacetylase, a histone methyltransferase (e.g., SUV39H1 or G9a (EHMT2)), a histone demethylase (e.g., Lys-specific histone demethylase 1 (LSD1)), a DNA methyltransferase (e.g., DNMT3a or DNMT3a-DNMT3L), a DNA demethylase (e.g., TET1 catalytic domain or TDG), or fragment thereof.
35. The method of any of the preceding paragraphs, wherein the modulator of gene expression is fused to the C-terminus, N-terminus, or both, of the dCas9 molecule.
36. The method of any of the preceding paragraphs, wherein the modulator of gene expression is fused to the dCas9 molecule directly.
37. The method of any of paragraphs 1-34, wherein the modulator of gene expression is fused to the dCas9 molecule indirectly, e.g., via a non-modulator or a linker, or a second modulator.
38. The method of any of the preceding paragraphs, wherein a plurality of modulators of gene expression, e.g., two or more identical, substantially identical, or different modulators, are fused to the dCas9 molecule.
39. The method of any of the preceding paragraphs, wherein the fusion molecule further comprises a nuclear localization sequence.
40. The method of paragraph 39, wherein one or more nuclear localization sequences are fused to the C-terminus, N-terminus, or both, of the dCas9 molecule, e.g., directly or indirectly, e.g., via a linker.
41. The method of paragraph 40, wherein the one or more nuclear localization sequences comprise the amino acid sequence of SEQ ID NO: 37 or 38, a sequence substantially identical (e.g., at least 80%, 85%, 90%, 92%, 95%, 97%, 98%, 99% or higher identical) to SEQ ID NO: 37 or 38, or a sequence having one, two, three, four, five or more changes, e.g., amino acid substitutions, insertions, or deletions, relative to SEQ ID NO: 37 or 38, or any fragment thereof.
42. The method of any of the preceding paragraphs, wherein the fusion molecule comprises the amino acid sequence of SEQ ID NO: 39, 40, or 41, a sequence substantially identical (e.g., at least 80%, 85%, 90%, 92%, 95%, 97%, 98%, 99% or higher identical) to SEQ ID NO: 39, 40, or 41, or a sequence having one, two, three, four, five or more changes, e.g., amino acid substitutions, insertions, or deletions, relative to SEQ ID NO: 39, 40, or 41, or any fragment thereof.
43. The method of any of the preceding paragraphs, wherein the nucleic acid that encodes the fusion molecule comprises the sequence of SEQ ID NO: 23, a sequence substantially identical (e.g., at least 80%, 85%, 90%, 92%, 95%, 97%, 98%, 99% or higher identical) to SEQ ID NO: 23, or a sequence having one, two, three, four, five or more changes, e.g., substitutions, insertions, or deletions, relative to SEQ ID NO: 23, or any fragment thereof.
44. The method of any of the preceding paragraphs, wherein the gRNA comprises a unimolecular gRNA.
45. The method of any of paragraphs 1-43, wherein the gRNA comprises a bimolecular gRNA.
46. The method of any of the preceding paragraphs, wherein the gRNA comprises a gRNA sequence described herein.
47. The method of any of the preceding paragraphs, wherein gene expression is modulated in a cell, tissue, or organ described herein, e.g., Table 2 or 3.
48. The method of any of the preceding paragraphs, wherein gene expression is modulated in the liver.
49. The method of any of the preceding paragraphs, wherein the modulation is sufficient to alter a function of the gene, or a symptom of a disorder associated with the gene, as described herein, e.g., in Table 2 or 3.
50. The method of any of the preceding paragraphs, wherein the modulation comprises modulation of transcription.
51. The method of any of the preceding paragraphs, wherein the modulation comprises down-regulation of transcription.
52. The method of any of the preceding paragraphs, wherein the modulation comprises up-regulation of transcription.
53. The method of any of the preceding paragraphs, wherein the modulation comprises modulating the temporal pattern of expression of the gene.
54. The method of any of the preceding paragraphs, wherein the modulation comprises modulating the spatial pattern of expression of the gene.
55. The method of any of the preceding paragraphs, wherein the modulation comprises modulating a post-transcriptional or co-transcriptional modification, e.g., splicing, 5′ capping, 3′ cleavage, 3′ polyadenylation, or RNA export.
56. The method of any of the preceding paragraphs, wherein the modulation comprises modulating the expression of an isoform, e.g., an increase or decrease in the expression of an isoform, the increase or decrease in the expression of a first isoform over a second isoform.
57. The method of any of the preceding paragraphs, wherein the modulation comprises modulating chromatin structure, e.g., increasing or decreasing methylation, acetylation, phosphorylation, or ubiquitination, e.g., at a preselected site, or altering the spatial pattern, cell specificity, or temporal occurrence of methylation, acetylation, phosphorylation, or ubiquitination.
58. The method of any of the preceding paragraphs, wherein the modulation comprises modulating a post-translational modification (e.g., indirectly), e.g., glycosylation, lipidation, acetylation, phosphorylation, amidation, hydroxylation, methylation, ubiquitination, sulfation, nitrosylation, or proteolysis.
59. The method of any of the preceding paragraphs, wherein the modulation does not comprise cleaving the subject's DNA.
60. The method of any of the preceding paragraphs, wherein the modulation comprises an inducible modulation.
61. The method of any of the preceding paragraphs, wherein the gene is selected from Table 2, optionally wherein the method down-regulates the expression of the gene.
62. The method of any of paragraphs 1-60, wherein the gene is selected from Table 3, optionally wherein the method up-regulates the expression of the gene.
63. The method of any of the preceding paragraphs, wherein the gene comprises PCSK9.
64. The method of any of the preceding paragraphs, wherein the dCas9 molecule does not cleave the genome of the subject.
65. A method of modulating expression of a gene, in vivo, in a subject comprising administering to, or providing in, the subject:
(a)(ii) a nucleic acid that encodes a fusion molecule (e.g., a fusion molecule described herein) comprising a sequence comprising an S. aureus dCas9 molecule fused to a KRAB molecule; and
(b)(ii) a nucleic acid that encodes a gRNA (e.g., a gRNA described herein) which targets the fusion molecule to the gene, and
wherein one or both of (a)(i) and (b)(ii) are packaged in an AAV vector.
66. The method of paragraph 65, wherein the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.
67. The method of paragraph 65 or 66, wherein the gRNA comprises a gRNA sequence described herein.
68. The method of any of paragraphs 65-67, wherein the gene is selected from Table 2 or 3.
69. The method of any of paragraphs 65-68, wherein the gene comprises PCSK9.
70. The method of any of paragraphs 65-69, wherein (a)(ii) and (b)(ii) are packaged in different AAV vectors.
71. The method of any of paragraphs 65-70, wherein (a)(ii) and (b)(ii) are packaged in the same AAV vector.
72. A pharmaceutical composition, or unit dosage form, comprising, in an amount sufficient for modulating a gene in a human subject, or in an amount sufficient for a therapeutic effect in a human subject,
(a)(ii) a nucleic acid that encodes a fusion molecule (e.g., a fusion molecule described herein) comprising a sequence comprising a dCas9 molecule, e.g., an S. aureus dCas9 molecule, fused to a modulator of gene expression (e.g., a modulator described herein); and/or
(b)(ii) a nucleic acid that encodes a gRNA which targets the fusion molecule to the gene,
wherein one or both of (a)(ii) and (b)(ii) are packaged in a viral vector, e.g., an AAV vector.
73. The pharmaceutical composition, or unit dosage form, of paragraph 72, wherein the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.
74. The pharmaceutical composition, or unit dosage form, of paragraph 72 or 73, wherein the gRNA comprises a gRNA sequence described herein.
75. The pharmaceutical composition, or unit dosage form, of any of paragraphs 72-74, wherein the gene is selected from Table 2 or 3.
76. The method of any of paragraphs 72-75, wherein the gene comprises PCSK9.
77. The pharmaceutical composition, or unit dosage form, of any of paragraphs 72-76, wherein (a)(ii) and (b)(ii) are packaged in the same viral vector, e.g., an AAV vector.
78. The pharmaceutical composition, or unit dosage form, of any of paragraphs 72-77, wherein (a)(ii) and (b)(ii) are packaged in different viral vectors, e.g., AAV vectors.
79. The pharmaceutical composition, or unit dosage form, of any of paragraphs 72-78, wherein the viral vector (e.g., AAV vector) comprising (a)(ii), and the viral vector (e.g., AAV vector) comprising (b)(ii), are provided in separate containers.
80. The pharmaceutical composition, or unit dosage form, of any of paragraphs 72-79, wherein the viral vector (e.g., AAV vector) comprising (a)(ii) and the viral vector (e.g., AAV vector) comprising (b)(ii), are provided in the same container.
81. The pharmaceutical composition, or unit dosage form, of any of paragraphs 72-80, which is formulated for administration, e.g., oral, parenteral, sublingual, transdermal, rectal, transmucosal, topical, intrapleural, intravenous, intraarterial, intraperitoneal, subcutaneous, intramuscular, intranasal intrathecal, or intraarticular administration, or administration via inhalation or via buccal administration, or any combination thereof, to the subject.
82. The pharmaceutical composition, or unit dosage form, of any of paragraphs 72-81, which is formulated for intravenous administration to the subject.
83. The pharmaceutical composition, or unit dosage form, of any of paragraphs 72-82, which is disposed in a device suitable for administration, e.g., oral, parenteral, sublingual, transdermal, rectal, transmucosal, topical, intrapleural, intravenous, intraarterial, intraperitoneal, subcutaneous, intramuscular, intranasal intrathecal, or intraarticular administration, or administration via inhalation or via buccal administration, or any combination thereof, to the subject.
84. The pharmaceutical composition, or unit dosage form, of any of paragraphs 72-83, which is disposed in a device suitable for intravenous administration to the subject.
85. The pharmaceutical composition, or unit dosage form, of any of paragraphs 72-84, which is disposed in a volume of at least 1, 2, 5, 10, 15, 20, 25, 30, 40, 50, 60, 70, 80, 90, 100, 150, 200, 250, 300, 400, or 500 ml.
86. The pharmaceutical composition, or unit dosage form, of any of paragraphs 72-85, wherein the nucleic acid of (a)(ii) comprises DNA.
87. The pharmaceutical composition, or unit dosage form, of any of paragraphs 72-86, wherein the nucleic acid of (b)(ii) comprises DNA.
88. The pharmaceutical composition, or unit dosage form, of paragraphs 72-85 or 87, wherein the nucleic acid of (a)(ii) comprises RNA.
89. The pharmaceutical composition, or unit dosage form, of paragraphs 72-86 or 88, wherein the nucleic acid of (b)(ii) comprises RNA.
90. The pharmaceutical composition, or unit dosage form, of paragraphs 72-89, wherein the dCas9 molecule comprises a gRNA binding domain of a Cas9 molecule.
91. The pharmaceutical composition, or unit dosage form, of paragraphs 72-90, wherein the dCas9 molecule comprises one, two or all of: a Rec1 domain, a bridge helix domain, or a PAM interacting domain, of a Cas9 molecule.
92. The pharmaceutical composition, or unit dosage form, of paragraphs 72-91, wherein the dCas9 molecule is a mutant of a wild-type Cas9 molecule, e.g., in which the Cas9 nuclease activity is inactivated.
93. The pharmaceutical composition, or unit dosage form, of paragraphs 72-90, wherein the dCas9 molecule comprises a mutation that inactivates a Cas9 nuclease activity, e.g., a mutation in a DNA-cleavage domain of a Cas9 molecule.
94. The pharmaceutical composition, or unit dosage form, of paragraphs 72-93, wherein the dCas9 molecule comprises a mutation that inactivates a Cas9 nuclease activity, e.g., a mutation in a RuvC domain and/or a mutation in a HNH domain.
95. The pharmaceutical composition, or unit dosage form, of paragraphs 72-94, wherein the dCas9 molecule comprises a Staphylococcus aureus dCas9 molecule, a Streptococcus pyogenes dCas9 molecule, a Campylobacter jejuni dCas9 molecule, a Corynebacterium diphtheria dCas9 molecule, a Eubacterium ventriosum dCas9 molecule, a Streptococcus pasteurianus dCas9 molecule, a Lactobacillus farciminis dCas9 molecule, a Sphaerochaeta globus dCas9 molecule, an Azospirillum (e.g., strain B510) dCas9 molecule, a Gluconacetobacter diazotrophicus dCas9 molecule, a Neisseria cinerea dCas9 molecule, a Roseburia intestinalis dCas9 molecule, a Parvibaculum lavamentivorans dCas9 molecule, a Nitratifractor salsuginis (e.g., strain DSM 16511) dCas9 molecule, a Campylobacter lari (e.g., strain CF89-12) dCas9 molecule, or a Streptococcus thermophilus (e.g., strain LMD-9) dCas9 molecule.
96. The pharmaceutical composition, or unit dosage form, of paragraphs 72-95, wherein the dCas9 molecule comprises an S. aureus dCas9 molecule, e.g., comprising an S. aureus dCas9 sequence described herein.
97. The pharmaceutical composition, or unit dosage form, of paragraph 96, wherein the S. aureus dCas9 molecule comprises a mutation at an amino acid position, corresponding to position 10, 580, or both (e.g., D10A, N580A, or both), relative to a wild-type S. aureus dCas9 molecule, numbered according to SEQ ID NO: 25.
98. The pharmaceutical composition, or unit dosage form, of paragraph 96, wherein the S. aureus dCas9 molecule comprises the amino acid sequence of SEQ ID NO: 35 or 36, a sequence substantially identical (e.g., at least 80%, 85%, 90%, 92%, 95%, 97%, 98%, 99% or higher identical) to SEQ ID NO: 35 or 36, or a sequence having one, two, three, four, five or more changes, e.g., amino acid substitutions, insertions, or deletions, relative to SEQ ID NO: 35 or 36, or any fragment thereof.
99. The pharmaceutical composition, or unit dosage form, of any of paragraphs 72-95, wherein the dCas9 molecule comprises an S. pyogenes dCas9 molecule, e.g., comprising an S. pyogenes dCas9 sequence described herein.
100. The pharmaceutical composition, or unit dosage form, of paragraph 99, wherein the S. pyogenes dCas9 molecule comprises a mutation at an amino acid position, corresponding to position 10, 840, or both (e.g., D10A, H840A, or both), relative to a wild-type S. pyogenes dCas9 molecule, numbered according to SEQ ID NO: 24.
101. The pharmaceutical composition, or unit dosage form, of paragraphs 72-100, wherein the dCas9 molecule is less than 1400, 1300, 1200, 1100, 1000, 900, 800, 700, 600, or 500 amino acids in length.
102. The pharmaceutical composition, or unit dosage form, of paragraphs 72-101, wherein the dCas9 molecule is 500-1300, 600-1200, 700-1100, 800-1000, 500-1200, 500-1000, 500-800, 500-600, 1000-1200, 800-1200, or 600-1200 amino acids in length.
103. The pharmaceutical composition, or unit dosage form, of paragraphs 72-102, wherein the dCas9 molecule has a size that is less than 90%, 80%, 70%, 60%, 50%, 40%, or 30% of the size of a wild-type Cas9 molecule, e.g., a wild-type S. pyogenes Cas9 molecule or a wild-type S. aureus dCas9 molecule.
104. The pharmaceutical composition, or unit dosage form, of any of paragraphs 72-103, wherein modulator of gene expression comprises a modulator of gene expression described herein.
105. The pharmaceutical composition, or unit dosage form, of any of paragraphs 72-104, wherein modulator of gene expression comprises a KRAB molecule, e.g., comprising the sequence of SEQ ID NO: 34, a sequence substantially identical (e.g., at least 80%, 85%, 90%, 92%, 95%, 97%, 98%, 99% or higher identical) to SEQ ID NO: 34, or a sequence having one, two, three, four, five or more changes, e.g., amino acid substitutions, insertions, or deletions, relative to SEQ ID NO: 34, or any fragment thereof.
106. The pharmaceutical composition, or unit dosage form, of any of paragraphs 72-105, wherein the gRNA comprises a unimolecular gRNA.
107. The pharmaceutical composition, or unit dosage form, of any of paragraphs 72-105, wherein the gRNA comprises a bimolecular gRNA. 108. The pharmaceutical composition, or unit dosage form, of any of paragraphs 72-107, wherein the gRNA comprises a gRNA sequence described herein.
109. The pharmaceutical composition, or unit dosage form, of any of paragraphs 72-108, wherein gene expression is modulated in a cell, tissue, or organ described herein, e.g., Table 2 or 3.
110. The pharmaceutical composition, or unit dosage form, of any of paragraphs 72-109, wherein gene expression is modulated in the liver.
111. The pharmaceutical composition, or unit dosage form, of any of paragraphs 72-110, wherein the modulation is sufficient to alter a function of the gene, or a symptom of a disorder associated with the gene, as described herein, e.g., in Table 2 or 3.
112. The pharmaceutical composition, or unit dosage form, of any of paragraphs 72-111, wherein the modulation comprises modulation of transcription.
113. The pharmaceutical composition, or unit dosage form, of any of paragraphs 72-112, wherein the modulation comprises down-regulation of transcription.
114. The pharmaceutical composition, or unit dosage form, of any of paragraphs 72-113, wherein the modulation comprises up-regulation of transcription.
115. The pharmaceutical composition, or unit dosage form, of any of paragraphs 72-114, wherein the modulation comprises modulating the temporal pattern of expression of the gene.
116. The pharmaceutical composition, or unit dosage form, of any of paragraphs 72-115, wherein the modulation comprises modulating the spatial pattern of expression of the gene.
117. The pharmaceutical composition, or unit dosage form, of any of paragraphs 72-116, wherein the modulation comprises modulating a post-transcriptional or co-transcriptional modification, e.g., splicing, 5′ capping, 3′ cleavage, 3′ polyadenylation, or RNA export.
118. The pharmaceutical composition, or unit dosage form, of any of paragraphs 72-117, wherein the modulation comprises modulating the expression of an isoform, e.g., an increase or decrease in the expression of an isoform, the increase or decrease in the expression of a first isoform over a second isoform.
119. The pharmaceutical composition, or unit dosage form, of any of paragraphs 72-118, wherein the modulation comprises modulating chromatin structure, e.g., increasing or decreasing methylation, acetylation, phosphorylation, or ubiquitination, e.g., at a preselected site, or altering the spatial pattern, cell specificity, or temporal occurrence of methylation, acetylation, phosphorylation, or ubiquitination.
120. The pharmaceutical composition, or unit dosage form, of any of paragraphs 72-119, wherein the modulation comprises modulating a post-translational modification (e.g., indirectly), e.g., glycosylation, lipidation, acetylation, phosphorylation, amidation, hydroxylation, methylation, ubiquitination, sulfation, nitrosylation, or proteolysis.
121. The pharmaceutical composition, or unit dosage form, of any of paragraphs 72-120, wherein the gene is selected from Table 2, optionally wherein the method down-regulates the expression of the gene.
122. The pharmaceutical composition, or unit dosage form, of any of paragraphs 72-120, wherein the gene is selected from Table 3, optionally wherein the method up-regulates the expression of the gene.
123. The pharmaceutical composition, or unit dosage form, of any of paragraphs 72-122, wherein the gene comprises PCSK9.
124. The pharmaceutical composition, or unit dosage form, of any of paragraphs 72-123, wherein the dCas9 does not cleave the genome of the subject.
125. A pharmaceutical composition, or unit dosage form, comprising, in an amount sufficient for modulating a gene in a human subject, or in an amount sufficient for a therapeutic effect in a human subject,
(a)(ii) a nucleic acid that encodes a fusion molecule comprising a sequence comprising an S. aureus dCas9 molecule fused to a KRAB molecule; and/or
(b)(ii) a nucleic acid that encodes a gRNA which targets the fusion molecule to the gene,
wherein one or both of (a)(ii) and (b)(ii) are packaged in a viral vector, e.g., an AAV vector.
126. The pharmaceutical composition, or unit dosage form, of paragraph 125, wherein the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.
127. The pharmaceutical composition, or unit dosage form, of paragraph 125 or 126, wherein the gRNA comprises a gRNA sequence described herein.
128. The pharmaceutical composition, or unit dosage form, of any of paragraphs 125-127, wherein the gene is selected from Table 2 or 3.
129. The pharmaceutical composition, or unit dosage form, of any of paragraphs 125-128, wherein the gene comprises PCSK9.
130. The pharmaceutical composition, or unit dosage form, of any of paragraphs 125-129, wherein (a)(ii) and (b)(ii) are packaged in different AAV vectors.
131. The pharmaceutical composition, or unit dosage form, of any of paragraphs 125-130, wherein (a)(ii) and (b)(ii) are packaged in the same AAV vector.
132. A viral vector comprising:
(a)(ii) a nucleic acid that encodes a fusion molecule (e.g., a fusion molecule described herein) comprising a sequence comprising a dCas9 molecule (e.g., a dCas9 molecule described herein), e.g., an S. aureus dCas9 molecule, fused to a modulator of gene expression (e.g., a modulator described herein); and/or
(b)(ii) a nucleic acid that encodes a gRNA (e.g., a gRNA described herein) which targets the fusion molecule to a gene (e.g., a gene described herein).
133. The viral vector of paragraph 132, which is an AAV vector.
134. The viral vector of paragraph 132 and 133, comprising:
(a)(ii) a nucleic acid that encodes a fusion molecule comprising a sequence comprising an S. aureus dCas9 molecule fused to a KRAB molecule; and
(b)(ii) a nucleic acid that encodes a gRNA which targets the fusion molecule to PCSK9,
wherein one or both of (a)(ii) and (b)(ii) are packaged in an AAV vector.
135. The viral vector of any of paragraphs 132-134, wherein the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.
136. The viral vector of any of paragraphs 132-135, wherein the gRNA comprises a gRNA sequence described herein.
137. The viral vector of any of paragraphs 132-136, wherein the gene is selected from Table 2 or 3.
138. The viral vector of any of paragraphs 132-137, wherein the gene comprises PCSK9.
139. A method of treating a disorder, comprising administering to a subject:
(a)(ii) a nucleic acid that encodes a fusion molecule (e.g., a fusion molecule described herein) comprising a sequence comprising a dCas9 molecule (e.g., a dCas9 molecule) fused to a modulator of gene expression (e.g., a modulator describe herein); and
(b)(ii) a nucleic acid that encodes a gRNA (e.g., a gRNA described herein) which targets the fusion molecule to a gene associated with the disorder,
thereby treating the disorder.
140. The method of paragraph 139, wherein the disorder is selected from Table 2 or 3.
141. The method of paragraph 139 or 140, wherein the gene is selected from Table 2 or 3.
142. The method of any of paragraphs 139-140, wherein one or both of (a)(ii) and (b)(ii) are provided in an AAV vector.
143. A method of treating a cardiovascular disease, comprising administering to a subject:
(a)(ii) a nucleic acid that encodes a fusion molecule (e.g., a fusion molecule described herein) comprising a sequence comprising a dCas9 molecule (e.g., a dCas9 molecule described herein) fused to a modulator of gene expression (e.g., a modulator describe herein); and
(b)(ii) a nucleic acid that encodes a gRNA (e.g., a gRNA described herein) which targets the fusion molecule to a PCSK9 gene,
thereby treating the cardiovascular disease.
144. The method of paragraph 143, wherein the dCas9 molecule is an S. aureus dCas9 molecule.
145. The method of paragraph 143 or 144, wherein the fusion molecule comprises a sequence described herein, e.g., the amino acid sequence of SEQ ID NO: 34, 35, 36, 37, 38, 39, 40, or 41, a sequence substantially identical (e.g., at least 80%, 85%, 90%, 92%, 95%, 97%, 98%, 99% or higher identical) to SEQ ID NO: 34, 35, 36, 37, 38, 39, 40, or 41, or a sequence having one, two, three, four, five or more changes, e.g., amino acid substitutions, insertions, or deletions, relative to SEQ ID NO: 34, 35, 36, 37, 38, 39, 40, or 41, or any fragment thereof.
146. The method of any of paragraphs 143-145, wherein the gRNA comprises a gRNA sequence described herein.
147. The method of any of paragraphs 143-146, wherein one or both of (a)(ii) and (b)(ii) are provided in an AAV vector.

The following examples are provided by way of illustration and not by way of limitation.

### EXAMPLES

1. In Vivo Transcriptional Repression of Endogenous Genes Using S. aureus Cas9-Based Repressors.

### 1.1 Synopsis

RNA-guided dCas9-KRAB repressors have demonstrated promise in cell culture models for silencing target gene expression efficiently and specifically. An exciting application of this technology would be to study gene regulation in development and disease in animal models and to design novel gene therapies. However, a technology to deliver CRISPR/Cas9-based gene repressors in vivo has not been developed. AAV vectors have been used as a delivery platform for CRISPR/Cas9 nuclease components for in vivo studies and therapeutic applications (Ran, F. A. et al. Nature 520, 186-91 (2015), incorporated by reference herein in its entirety). Recently, a smaller Cas9 nuclease protein derived from S. aureus was described for AAV delivery and in vivo gene editing (Ran, F. A. et al. Nature 520, 186-91 (2015)). In this example, a KRAB repressor motif was fused to S. aureus nuclease-null dCas9 (dSaCas9), thereby generating a programmable RNA-guided repressor for in vivo gene regulation. dSaCas9-KRAB repressors efficiently silenced a reporter luciferase gene in primary fibroblasts and the myostatin receptor Acvr2b in a mouse myoblast cell line. When delivered intramuscularly via an AAV9 dual-vector expression system, dSaCas9-KRAB and Acvr2b gRNA were efficiently expressed in the injected tibialis anterior, heart, and liver tissues of adult wild-type mice. No appreciable silencing of Acvr2b was achieved in skeletal muscle, but dSaCas9-KRAB was biologically active and significantly silenced Acvr2b expression in heart and liver when delivered with a target guide RNA molecule. This gene delivery system can be customized to target any endogenous gene, enabling potent and stable gene repression in animal models and for therapeutic applications.

### 1.2 Introduction

RNA-guided gene regulation with the CRISPR/Cas9 system has enabled functional genomics studies in cell culture systems (Kearns, N. A. et al. Nat Methods (2015); Gilbert, L. A. et al. Cell 159, 647-61 (2014); Thakore, P. I. et al. Nat Methods 12, 1143-9 (2015); Konermann, S. et al. Nature 517, 583-8 (2015), incorporated by reference herein in their entirety). The potency and specificity of dCas9-KRAB epigenetic repressors, in particular, are promising for loss-of-function studies and guiding cell phenotype in vitro (Thakore, P. I., et al. Nat Methods 13, 127-37 (2016); Gilbert, L. A. et al. Cell 159, 647-61 (2014); Thakore, P. I. et al. Nat Methods 12, 1143-9 (2015), incorporated by reference herein in their entirety). Adapting programmable transcriptional modulators for use in vivo would allow for the study of gene regulation in complex organisms and enable the development of therapies to address aberrant gene regulation in disease.

The large packaging capacity of lentiviral vectors, a commonly used method to stably deliver CRISPR/Cas9 components in vitro, can accommodate the 4.2 kb S. pyogenes Cas9, epigenetic modulator fusions, a single gRNA, and associated regulatory elements required for expression. While efficacious for in vitro delivery, under certain circumstances, lentiviral delivery is typically not suitable for in vivo gene regulation due to concerns for insertional mutagenesis. Adeno-associated viral (AAV) vectors are a promising gene delivery vehicle as they provide stable episomal gene expression with minimal integration and have been extensively engineered to target a variety of tissue types (Asokan, A., et al. Mol Ther 20, 699-708 (2012), incorporated by reference herein in its entirety). However, the packaging capacity of AAV is limited to 4.5 kb, precluding delivery of the 4.2 kb S. pyogenes dCas9 DNA-binding domain, KRAB repressor motif, and associated regulatory elements. A smaller 3.2 kb Cas9 nuclease derived from S. aureus (SaCas9) has recently been identified and adapted for genome editing in vivo in the liver and skeletal muscle (Ran, F. A. et al. Nature 520, 186-91 (2015); Nelson, C. E. et al. Science 351, 403-7 (2016); Tabebordbar, M. et al. Science 351, 407-11 (2016), incorporated by reference herein in their entirety). A SaCas9-based transcriptional repressor was generated for AAV-based delivery and silencing of endogenous genes in vivo.

The SaCas9-based transcriptional repressor was tested in vitro for silencing a luciferase reporter gene in primary fibroblasts. For in vivo gene regulation, the myostatin receptor, Acvr2b, was targeted. Inhibiting the myostatin signaling pathway is a potential method for treating skeletal muscle degeneration. Myostatin is a secreted protein that acts as a negative regulator of skeletal muscle growth by binding the activin type II receptor (Acvr2b) and activating TGF-β signaling pathways (Lee, S. J. Annu Rev Cell Dev Biol 20, 61-86 (2004), incorporated by reference herein in its entirety). Knockout animal models of myostatin and Acvr2b demonstrate a double muscling phenotype (Lee, S. J. Annu Rev Cell Dev Biol 20, 61-86 (2004); Lee, S. J. et al. Proc Natl Acad Sci USA 109, E2353-60 (2012), incorporated by reference herein in its entirety). Blocking myostatin signaling through systemic administration of blocking antibodies or soluble Acvr2b receptors has been tested in clinical trials for the treatment of muscular dystrophy, but has thus far showed limited efficacy and safety concerns over adverse side effects (Wagner, K. R. et al. Ann Neurol 63, 561-71 (2008); Smith, R. C. & Lin, B. K. Curr Opin Support Palliat Care 7, 352-60 (2013), incorporated by reference herein in their entirety). A more targeted strategy to localize myostatin inhibition to skeletal muscle may increase the efficacy and safety of this strategy for treating muscle disorders.

An AAV9 two-vector system was designed for expressing SaCas9 repressors and targeting guide RNA (gRNA) molecule. AAV9 can provide stable and high transgene expression in skeletal and cardiac muscle (Asokan, A., et al. Mol Ther 20, 699-708 (2012); Zincarelli, C., et al. Mol Ther 16, 1073-80 (2008), incorporated by reference herein in their entirety) and is currently being evaluated in clinical trials for spinal muscular atrophy. When delivered intramuscularly in adult wild-type mice, SaCas9 repressors effected significant silencing of the endogenous Acvr2b gene in the heart and liver. These studies demonstrate that SaCas9-based repressors can regulate genes in animal models and will facilitate the development of gene-regulation based therapies.

### 1.3 Materials and Methods

### 1.3.1 Plasmid Constructs and AAV Design

An inactive version of SaCas9 (dSaCas9) was created by introducing D10A and N580A mutations (Ran, F. A. et al. Nature 520, 186-91 (2015), incorporated by reference herein in its entirety). dSaCas9 was cloned into a lentiviral vector driven by the human Ubiquitin C (hUbC) promoter, fused to a KRAB repressor motif, and linked to a puromycin resistance cassette via T2A ribosome skipping peptide. For sgRNA screening, the oligonucleotides containing protospacer sequences were synthesized (IDT-DNA), hybridized, phosphorylated, and inserted into a phU6-SaCas9 gRNA plasmid using BbsI sites. U6-gRNA cassettes were then cloned in reverse orientation upstream of the hUbC promoter in dSaCas9-KRAB lentiviral vectors for stable expression.

A Staphylococcus aureus Cas9 (SaCas9) AAV expression plasmid (Addgene #61592) was received as a gift from the Zhang lab (Ran, F. A. et al. Nature 520, 186-U98 (2015), incorporated by reference herein in its entirety). We replaced the nuclease-active SaCas9 with dSaCas9-KRAB. We also removed the C′ terminal 3×HA epitope tag and incorporated a single N′ terminal HA tag for tracking protein expression. For the AAV-U6 gRNA plasmid, a U6-Acvr2b gRNA cassette was cloned into a pTR-eGFP backbone replacing the CMV with the gRNA.

### 1.3.2 Cell Culture

C2C12s cells and HEK293T cells were obtained from the American Tissue Collection Center (ATCC) through the Duke University Cancer Center Facilities. Primary fibroblasts were harvested from the tail and ear of adult mice expressing a CAG-Luciferase-P2A-GFP cassette (Jackson Laboratories). C2C12 cells were maintained in DMEM supplemented with 20% FBS and 1% penicillin-streptomycin. HEK293T cells were cultured in DMEM supplemented with 10% FBS and 1% penicillin-streptomycin.

Mouse fibroblasts were cultured in DMEM supplemented with 10% FBS and 1% penicillin-streptomycin. All cell lines were cultured at 37 C with 5% CO₂.

### 1.3.3 Lentiviral Production

C2C12s and primary fibroblasts were transduced with lentivirus to stably express dSaCas9-KRAB and target gRNA molecules. To produce VSV-G pseudotyped lentivirus, HEK293T cells were plated at a density of 5.1e3 cells/cm² in high glucose DMEM supplemented with 10% FBS and 1% pencillin-streptomycin. The next day after seeding, cells in 10-cm plates were co-transfected with the appropriate dSaCas9-KRAB lentiviral expression plasmid (20 μg), the second-generation packaging plasmid psPAX2 (Addgene #12260, 15 μg), and the envelope plasmid pMD2.G (Addgene #12259, 6 μg) by calcium phosphate precipitation (Salmon, P. & Trono, D. Curr Protoc Neurosci Chapter 4, Unit 4 21 (2006), incorporated by reference herein in its entirety). After 14-20 hours, transfection medium was exchanged for 10 mL of fresh 293T medium. Conditioned medium containing lentivirus was collected 24 and 48 hours after the first media exchange. Residual producer cells were cleared from the lentiviral supernatant by filtration through 0.45 μm cellulose acetate filters and incubated overnight by incubation with Lenti-X. Concentrated virus was pelleted by centrifugation according to the manufacturer's protocol and resuspended at 20-fold concentration in PBS. Concentrated viral supernatant was snap-frozen in liquid nitrogen and stored at −80° C. for future use. For transduction, concentrated viral supernatant was diluted 1:20 with media. To facilitate transduction, the cationic polymer polybrene was added at a concentration of 4 μg/mL to the viral media. Non-transduced (NT) cells did not receive virus but were treated with polybrene as a control. The day after transduction, the medium was exchanged to remove the virus. Puromycin at 2 ug/mL (C2C12s) or 4 ug/mL (fibroblasts) was used to initiate selection for transduced cells approximately 48 hours after transduction.

### 1.3.4 AAV Production

ITRs were verified by Smal digest before production. AAV-dSaCas9-KRAB and AAV-U6 Acvr2b gRNA were used to generate AAV9 in two separate batches by the Gene Transfer Vector Core at Schepens Eye Research Institute, Massachusetts Eye and Ear. Titers were provided at 5.3×10¹³ vp/mL (AAV-dSaCas9-KRAB) and 1.6×10¹³ vp/mL (AAV-U6 Acvr2b gRNA).

### 1.3.5 Animal Studies

Animal studies were conducted with adherence to the guidelines for the care and use of laboratory animals of the National Institutes of Health (NIH). All the experiments with animals were approved by the Institutional Animal Care and Use Committee (IACUC) at Duke University. 6-8 week old C57Bl/6 mice (Jackson Labs) were anesthetized and maintained at 37° C. The right tibialis anterior muscle was prepared and injected with 30-40 μL of AAV solution (5.6×10¹¹-7.46×10¹¹ vp) or sterile PBS using a 30G needle. Mice were injected with a saline control, a 5e11 vp dose AAV-dSaCas9-KRAB alone, or a 1:1 mixture of 1e12 total dose of AAV-dSaCas9-KRAB and AAV-U6 Acvr2b gRNA. At 4 and 8 weeks post-injection, mice were euthanized by CO₂ inhalation and tissue was collected into RNALater® (Life Technologies) for DNA and RNA or snap-frozen for protein analysis.

1.3.6 qRT-PCR

Cells were harvested for total RNA isolation using the RNeasy Plus RNA isolation kit (Qiagen). Tissue samples were stored in RNALater (Ambion) and total RNA was isolated using the RNA Universal Plus Kit (Qiagen). cDNA synthesis was performed using the SuperScript VILO cDNA Synthesis Kit (Invitrogen). For genomic qPCR experiments, genomic DNA from tissue samples was isolated using a Blood and Tissue Kit (Qiagen). Quantitative real-time PCR (qRT-PCR) using QuantIT Perfecta Supermix was performed with the CFX96 Real-Time PCR Detection System (Bio-Rad) with the oligonucleotide primers optimized for 90-110% amplification efficiency. The results are expressed as fold-increase mRNA expression of the gene of interest normalized to Gapdh expression by the ΔΔCt method.

### 1.3.7 Western Blot

Cells or minced tissue were lysed in RIPA buffer (Sigma), and the BCA assay (Pierce) was performed to quantify total protein. Lysates were mixed with LDS sample buffer (Invitrogen) and boiled for 5 min; equal amounts of total protein were run in NuPAGE Novex 4-12% Bis-Tris polyacrylamide gels (Life Technologies) and transferred to nitrocellulose membranes. Nonspecific antibody binding was blocked with 5% nonfat milk in TBS-T (50 mM Tris, 150 mM NaCl and 0.1% Tween-20) for 30 min. The membranes were then incubated with primary antibody in 5% milk in TBS-T: rabbit anti-ACTRIIB diluted 1:1000 overnight at 4° C., anti-HA diluted 1:1000 for 60 min at room temperature, or rabbit anti-GAPDH diluted 1:5000 for 60 min at room temperature. Membranes labeled with primary antibodies were incubated with anti-mouse (Santa Cruz, SC-2005) or anti-rabbit HRP-conjugated antibody (Sigma-Aldrich, A6154) diluted 1:5000 for 60 min and washed with TBS-T for 60 min. Membranes were visualized using the Immun-Star WesternC Chemiluminescence Kit (Bio-Rad) and images were captured using a ChemiDoc XRS+ system and processed using ImageLab software (Bio-Rad).

### 1.4 Results

1.4.1 Generation of a Transcriptional Repressor from S. aureus Cas9

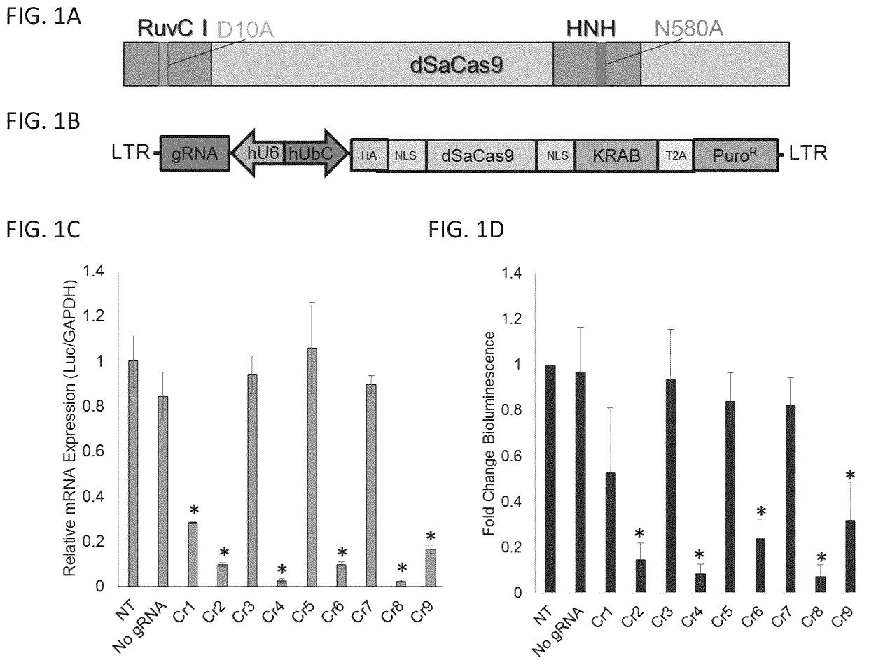

D10A and N580A mutations were introduced into the SaCas9 nuclease in order to abrogate catalytic activity and create a nuclease-null programmable DNA-binding domain (Ran, F. A. et al. Nature 520, 186-91 (2015), incorporated by reference herein in its entirety) (FIG. 1A). Fusion of a synthetic KRAB motif generated a dSaCas9 repressor. An N-terminal HA-tag was included to facilitate protein analysis and an N- and C-terminal nuclear localization sequence was included to enable trafficking of dSaCas9-KRAB into the cell nucleus.

For initial testing in vitro, dSaCas9-KRAB and single gRNAs were stably expressed using a lentiviral delivery system with puromycin selection (FIG. 1B). dSaCas9-KRAB was first tested in primary mouse fibroblasts expressing a luciferase reporter knocked in at chromosome 7 of the genome. Nine gRNAs to the synthetic CAG promoter driving transgene expression were designed, searching for base pair target sequences followed by the SaCas9 PAM, 5′ NNGRRT 3′ (SEQ ID NO: 1, wherein Nis any nucleotide, and R is G or A). Multiple gRNAs exhibited robust repression of luciferase expression via qPCR and Western 7 days after transduction of fibroblasts (FIGS. 1C and 1D). These results confirmed that dSaCas9-KRAB repressors were effective at silencing a reporter gene in vitro.

### 1.4.2 Silencing Endogenous Acvr2b in Myoblasts

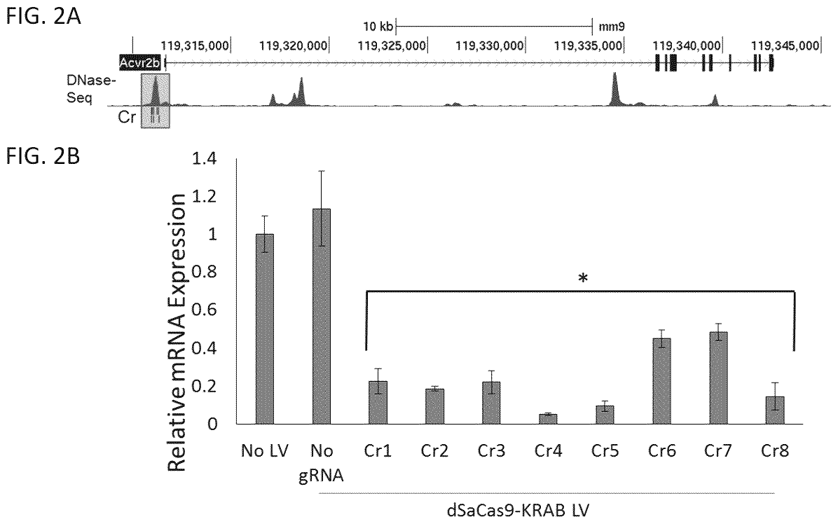

SaCas9-based repressors were targeted to the myostatin receptor Acvr2b in C2C12 mouse myoblasts. gRNAs were targeted to the DNase I hypersensitivity site (DHS) containing the transcription start site (TSS) of Acvr2b according to DNase-seq data on mouse skeletal muscle from the ENCODE project (Consortium, E. P. et al. Nature 489, 57-74 (2012), incorporated by reference herein in its entirety) (FIG. 2A). dSaCas9-KRAB and a single gRNA were stably expressed using a lentiviral delivery system, and multiple gRNAs effected potent repression of endogenous Acvr2b by qPCR 7 days after transduction and selection in C2C12s (FIG. 2B).

1.4.3 Transcriptional Repression of the Acvr2b Gene In Vivo with AAV Delivery of S. aureus Cas9 Repressors

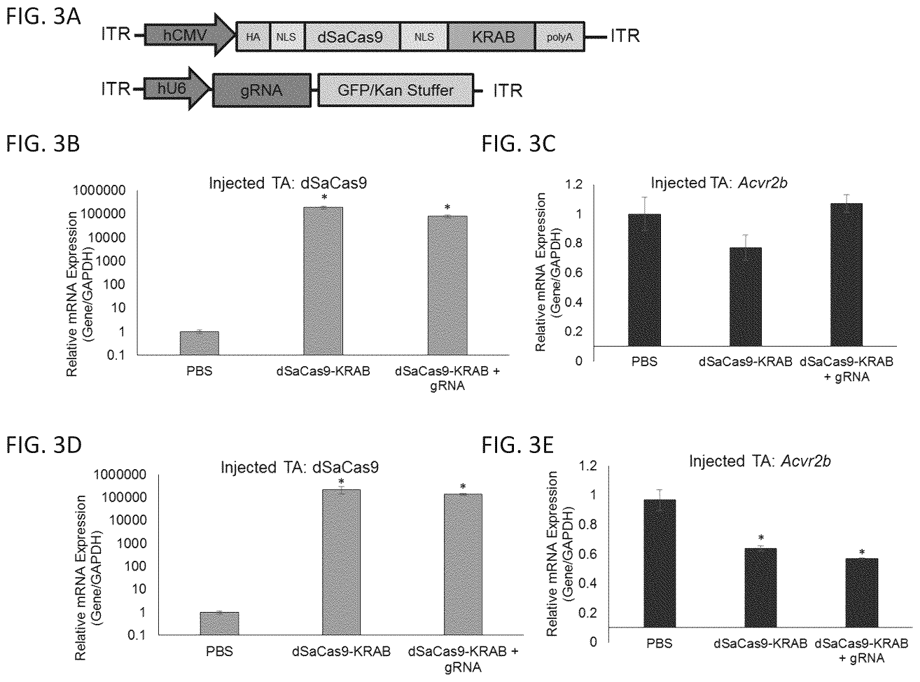

To accommodate the limited packaging capacity of AAV, a two-vector system was designed to deliver dSaCas9-KRAB and a single gRNA for targeted gene repression (FIG. 3A). AAV9 vectors expressing dSaCas9-KRAB and an Acvr2b gRNA were generated and purified by the Massachusetts General Hospital Ear and Eye Vector Core. The Cr4 Acvr2b gRNA was chosen for AAV in vivo studies. AAV9 is a muscle-tropic serotype capable of producing high levels of transgene expression (Zincarelli, C., et al. Mol Ther 16, 1073-80 (2008), incorporated by reference herein in its entirety).

Adult C57Bl/6 wild-type mice were injected in the tibialis anterior of the right limb with a mixture of AAV-dSaCas9-KRAB and AAV-Acvr2b-gRNA, at 5e11 vector genome copies delivered per AAV per limb. Age-matched controls received a PBS sham injection or AAV-dSaCas9-KRAB injection without gRNA. At 4 and 8 weeks post-transduction, dSaCas9-KRAB was steadily expressed via qPCR in the injected TA muscle (FIGS. 3B and 3D). Acvr2b expression was not significantly affected by delivery of dSaCas9-KRAB alone or dSaCas9-KRAB with Acvr2b gRNA at 4 weeks post-treatment (FIG. 3C). At 8 weeks post-AAV delivery, Acvr2b mRNA expression was significantly reduced compared to sham-injected muscles in both AAV treatment groups (FIG. 3E). However, targeting dSaCas9-KRAB with Acvr2b gRNA result in stronger repression than delivery of dSaCas9-KRAB alone.

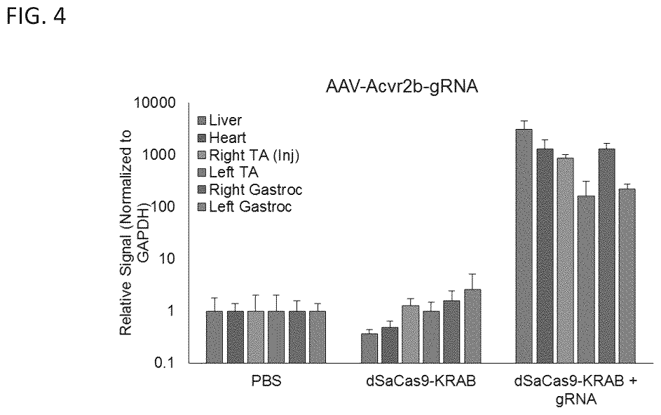

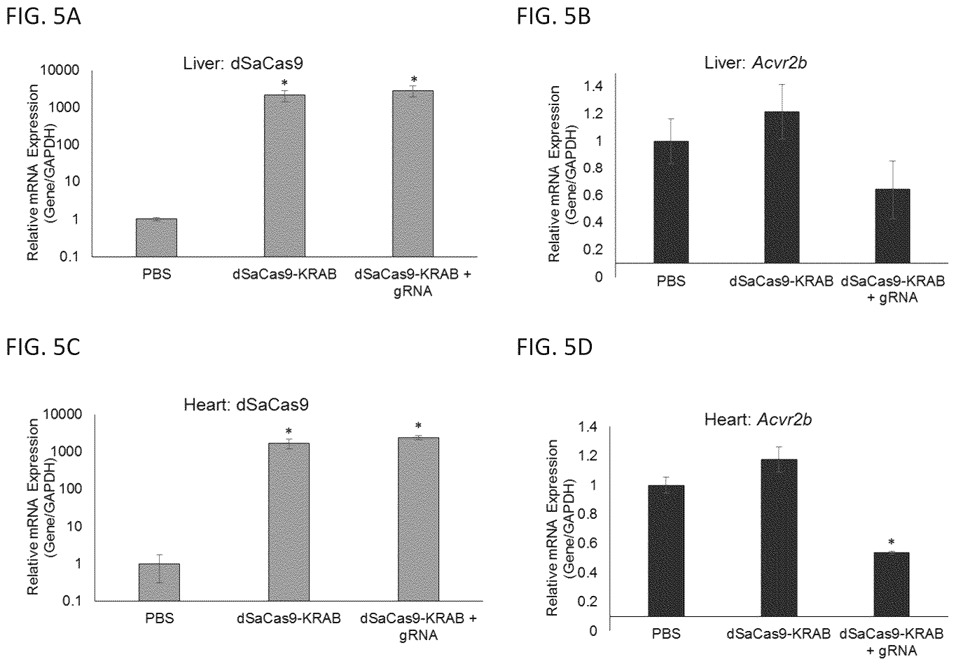

To determine if delivered AAV escaped the injected muscle and distributed systemically, vector genome signal was quantified in the liver, heart, and tibialis anterior muscles of treated mice at 8 weeks post-transduction. For AAV-Acvr2b-gRNA, the highest vector genome signals were found in the liver, heart, the right gastrocnemius muscle, and the injected tibialis anterior muscle (FIG. 4). Various AAV serotypes demonstrate tropism for the liver, and AAV9 can efficiently transduce cardiac muscle (Asokan, A., et al. Mol Ther 20, 699-708 (2012); Zincarelli, C., et al. Mol Ther 16, 1073-80 (2008), incorporated by reference herein in their entirety). dSaCas9-KRAB was expressed in the liver and heart at 4 and 8 weeks post-transduction via qPCR (FIG. 5). At 8 weeks post-transduction, Acvr2b expression in the heart was reduced by ˜50% with delivery of dSaCas9-KRAB with gRNA. dSaCas9-KRAB alone did not have a significant effect on Acvr2b expression. Changes in Acvr2b expression in the liver were not statistically significant at 8 weeks post-transduction. These results indicate that dSaCas9-KRAB is biologically active in vivo and AAV delivery is a promising method for achieving targeted repression in animal models.

### 1.5 Discussion

The efficiency and specificity of CRISPR/Cas9 gene silencing has shown great preclinical promise. In this example, a platform was presented to translate RNA-guided gene repression in vivo in a wild-type mouse model. dSaCas9-KRAB potently silenced reporter and endogenous genes in vitro, and AAV9 delivery of CRISPR/Cas9 components in an adult wild-type mouse model resulted in efficient silencing of the Acvr2b gene in the heart.

Muscle tissue contains large and multinucleated fibers and a progenitor population capable of proliferation and regeneration. These are all factors that may have contributed to the lack of repression observed in skeletal muscle. dSaCas9-KRAB repression in muscle may have limited by replication-mediated AAV dilution, diffusion of the repressor protein and delivered gRNA molecule along the myofiber, or inability of dSaCas9-KRAB to silence the majority of nuclei within a fiber. In contrast, cardiomyocytes of the heart are binucleated and post-mitotic, factors that may have contributed to the more efficient silencing observed in this tissue.

Interestingly, in some cases, it was observed that delivering dSaCas9-KRAB alone significantly downregulated Acvr2b expression. This unexpected biological effect may be related to potential host immune responses of high doses of AAV or expressing foreign SaCas9-based proteins in mouse tissue. An influx of immune cells or inflammatory responses could lead to gene expression changes in AAV-treated tissues and apparent silencing of the target gene.

The CRISPR/Cas9 platform is highly flexible, and the AAV delivery system developed in this example can easily be adapted to target other gene products. The extent of immune response to foreign Cas9 proteins and synthetic gRNA molecules, as well as the specificity of SaCas9-based gene regulation, can also be evaluated. A major determinant of off-site target binding is the presence of a PAM sequence, and thus the more stringent PAM requirement of SaCas9 compared to SpCas9 may be indicative of at least comparable levels of specificity for gene regulation. Lastly, minimal and tissue-specific promoters may enable implementation of a single AAV vector system for future in vivo gene regulation applications.

### 1.6. Appendix

1.6.1 Lentiviral S. aureus Cas9 KRAB-based repressor

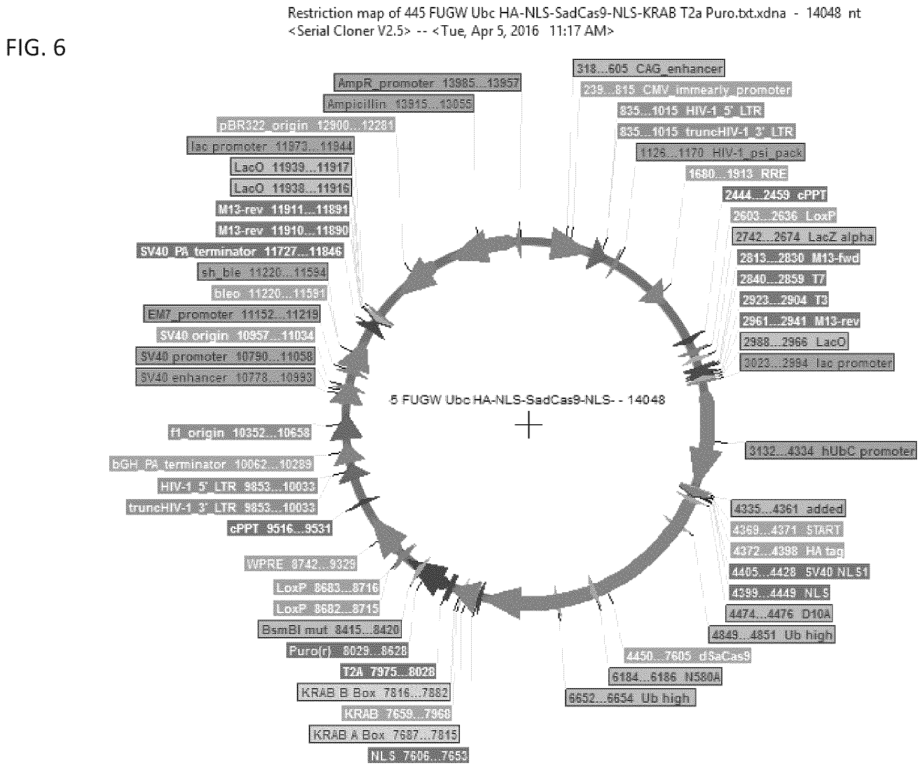

A restriction map of a lentiviral vector encoding S. aureus Cas9 KRAB-based repressor is shown in FIG. 6. SEQ ID NO: 2 provides the nucleic acid sequence of the lentivial vector encoding S. aureus Cas9 KRAB-based repressor.

SEQ ID NO: 2
GTCGACGGATCGGGAGATCTCCCGATCCCCTATGGTGCACTCTCAGTACA
ATCTGCTCTGATGCCGCATAGTTAAGCCAGTATCTGCTCCCTGCTTGTGT
GTTGGAGGTCGCTGAGTAGTGCGCGAGCAAAATTTAAGCTACAACAAGGC
AAGGCTTGACCGACAATTGCATGAAGAATCTGCTTAGGGTTAGGCGTTTT
GCGCTGCTTCGCGATGTACGGGCCAGATATACGCGTTGACATTGATTATT
GACTAGTTATTAATAGTAATCAATTACGGGGTCATTAGTTCATAGCCCAT
ATATGGAGTTCCGCGTTACATAACTTACGGTAAATGGCCCGCCTGGCTGA
CCGCCCAACGACCCCCGCCCATTGACGTCAATAATGACGTATGTTCCCAT
AGTAACGCCAATAGGGACTTTCCATTGACGTCAATGGGTGGAGTATTTAC
GGTAAACTGCCCACTTGGCAGTACATCAAGTGTATCATATGCCAAGTACG
CCCCCTATTGACGTCAATGACGGTAAATGGCCCGCCTGGCATTATGCCCA
GTACATGACCTTATGGGACTTTCCTACTTGGCAGTACATCTACGTATTAG
TCATCGCTATTACCATGGTGATGCGGTTTTGGCAGTACATCAATGGGCGT
GGATAGCGGTTTGACTCACGGGGATTTCCAAGTCTCCACCCCATTGACGT
CAATGGGAGTTTGTTTTGGCACCAAAATCAACGGGACTTTCCAAAATGTC
GTAACAACTCCGCCCCATTGACGCAAATGGGCGGTAGGCGTGTACGGTGG
GAGGTCTATATAAGCAGCGCGTTTTGCCTGTACTGGGTCTCTCTGGTTAG
ACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCT
GTTGTGTGACTCTGGTAACTAGAGATCCCTCAGACCCTTTTAGTCAGTGT
GGAAAATCTCTAGCAGTGGCGCCCGAACAGGGACTTGAAAGCGAAAGGGA
AACCAGAGGAGCTCTCTCGACGCAGGACTCGGCTTGCTGAAGCGCGCACG
GCAAGAGGCGAGGGGCGGCGACTGGTGAGTACGCCAAAAATTTTGACTAG
CGGAGGCTAGAAGGAGAGAGATGGGTGCGAGAGCGTCAGTATTAAGCGGG
GGAGAATTAGATCGCGATGGGAAAAAATTCGGTTAAGGCCAGGGGGAAAG
AAAAAATATAAATTAAAACATATAGTATGGGCAAGCAGGGAGCTAGAACG
ATTCGCAGTTAATCCTGGCCTGTTAGAAACATCAGAAGGCTGTAGACAAA
TACTGGGACAGCTACAACCATCCCTTCAGACAGGATCAGAAGAACTTAGA
TCATTATATAATACAGTAGCAACCCTCTATTGTGTGCATCAAAGGATAGA
GATAAAAGACACCAAGGAAGCTTTAGACAAGATAGAGGAAGAGCAAAACA
AAAGTAAGACCACCGCACAGCAAGCGGCCGCTGATCTTCAGACCTGGAGG
AGGAGATATGAGGGACAATTGGAGAAGTGAATTATATAAATATAAAGTAG
TAAAAATTGAACCATTAGGAGTAGCACCCACCAAGGCAAAGAGAAGAGTG
GTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGG
TACAGGCCAGACAATTATTGTCTGGTATAGTGCAGCAGCAGAACAATTTG
CTGAGGGCTATTGAGGCGCAACAGCATCTGTTGCAACTCACAGTCTGGGG
CATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACC
ACTGCTGTGCCTTGGAATGCTAGTTGGAGTAATAAATCTCTGGAACAGAT
TTGGAATCACACGACCTGGATGGAGTGGGACAGAGAAATTAACAATTACA
CAAGCTTAATACACTCCTTAATTGAAGAATCGCAAAACCAGCAAGAAAAG
AATGAACAAGAATTATTGGAATTAGATAAATGGGCAAGTTTGTGGAATTG
GTTTAACATAACAAATTGGCTGTGGTATATAAAATTATTCATAATGATAG
TAGGAGGCTTGGTAGGTTTAAGAATAGTTTTTGCTGTACTTTCTATAGTG
AATAGAGTTAGGCAGGGATATTCACCATTATCGTTTCAGACCCACCTCCC
AACCCCGAGGGGACCCGACAGGCCCGAAGGAATAGAAGAAGAAGGTGGAG
AGAGAGACAGAGACAGATCCATTCGATTAGTGAACGGATCGGCACTGCGT
GCGCCAATTCTGCAGACAAATGGCAGTATTCATCCACAATTTTAAAAGAA
AAGGGGGGATTGGGGGGTACAGTGCAGGGGAAAGAATAGTAGACATAATA
GCAACAGACATACAAACTAAAGAATTACAAAAACAAATTACAAAAATTCA
AAATTTTCGGGTTTATTACAGGGACAGCAGAGATCCAGTTTGGTTAatTA
AATAACTTCGTATAGCATACATTATACGAAGTTATGATAAGAGACGGTGG
TGgcgccgctacagggcgcgtcccattcgccattcaggctgcgcaactgt
tgggaagggcgatcggtgcgggcctcttcgctattacgccagctggcgaa
agggggatgtgctgcaaggcgattaagttgggtaacgccagggttttccc
agtcacgacgttgtaaaacgacggccagtgagcgcgcgtaatacgactca
ctatagggcgaattgggtaccgggccccccctcgaggtcctccagctttt
gttccctttagtgagggttaattgcgcgcttggcgtaatcatggtcatag
ctgtttcctgtgtgaaattgttatccgctcacaattccacacaacatacg
agccggaagcataaagtgtaaagcctggggtgcctaatgagtgagctaac
tcacattaattgcgttgcgctcactgcccgctttccaCTGCATGACGTCT
CCACAATTAatTAAgggtgcagcggcctccgcgccgggttttggcgcctc
ccgcgggcgcccccctcctcacggcgagcgctgccacgtcagacgaaggg
cgcaggagcgttcctgatccttccgcccggacgctcaggacagcggcccg
ctgctcataagactcggccttagaaccccagtatcagcagaaggacattt
taggacgggacttgggtgactctagggcactggttttctttccagagagc
ggaacaggcgaggaaaagtagtcccttctcggcgattctgcggagggatc
tccgtggggcggtgaacgccgatgattatataaggacgcgccgggtgtgg
cacagctagttccgtcgcagccgggatttgggtcgcggttcttgtttgtg
gatcgctgtgatcgtcacttggtgagttgcgggctgctgggctggccggg
gctttcgtggccgccgggccgctcggtgggacggaagcgtgtggagagac
cgccaagggctgtagtctgggtccgcgagcaaggttgccctgaactgggg
gttggggggagcgcacaaaatggcggctgttcccgagtcttgaatggaag
acgcttgtaaggcgggctgtgaggtcgttgaaacaaggtggggggcatgg
tgggcggcaagaacccaaggtcttgaggccttcgctaatgcgggaaagct
cttattcgggtgagatgggctggggcaccatctggggaccctgacgtgaa
gtttgtcactgactggagaactcgggtttgtcgtctggttgcgggggcgg
cagttatgcggtgccgttgggcagtgcacccgtacctttgggagcgcgcg
cctcgtcgtgtcgtgacgtcacccgttctgttggcttataatgcagggtg
gggccacctgccggtaggtgtgcggtaggcttttctccgtcgcaggacgc
agggttcgggcctagggtaggctctcctgaatcgacaggcgccggacctc
tggtgaggggagggataagtgaggcgtcagtttctttggtcggttttatg
tacctatcttcttaagtagctgaagctccggttttgaactatgcgctcgg
ggttggcgagtgtgttttgtgaagttttttaggcaccttttgaaatgtaa
tcatttgggtcaatatgtaattttcagtgttagactagTaaattgtccgc
taaattctggccgtttttggcttttttgttagacGAAGCTTGGGCTGCAG
GTCGACTctagagccaccatgtacccatacgatgttccagattacgctAT
GGCCCCAAAGAAGAAGCGGAAGGTCGGTATCCACGGAGTCCCAGCAGCCA
AGCGGAACTACATCCTGGGCCTGGCCATCGGCATCACCAGCGTGGGCTAC
GGCATCATCGACTACGAGACACGGGACGTGATCGATGCCGGCGTGCGGCT
GTTCAAAGAGGCCAACGTGGAAAACAACGAGGGCAGGCGGAGCAAGAGAG
GCGCCAGAAGGCTGAAGCGGCGGAGGCGGCATAGAATCCAGAGAGTGAAG
AAGCTGCTGTTCGACTACAACCTGCTGACCGACCACAGCGAGCTGAGCGG
CATCAACCCCTACGAGGCCAGAGTGAAGGGCCTGAGCCAGAAGCTGAGCG
AGGAAGAGTTCTCTGCCGCCCTGCTGCACCTGGCCAAGAGAAGAGGCGTG
CACAACGTGAACGAGGTGGAAGAGGACACCGGCAACGAGCTGTCCACCAA
AGAGCAGATCAGCCGGAACAGCAAGGCCCTGGAAGAGAAATACGTGGCCG
AACTGCAGCTGGAACGGCTGAAGAAAGACGGCGAAGTGCGGGGCAGCATC
AACAGATTCAAGACCAGCGACTACGTGAAAGAAGCCAAACAGCTGCTGAA
GGTGCAGAAGGCCTACCACCAGCTGGACCAGAGCTTCATCGACACCTACA
TCGACCTGCTGGAAACCCGGCGGACCTACTATGAGGGACCTGGCGAGGGC
AGCCCCTTCGGCTGGAAGGACATCAAAGAATGGTACGAGATGCTGATGGG
CCACTGCACCTACTTCCCCGAGGAACTGCGGAGCGTGAAGTACGCCTACA
ACGCCGACCTGTACAACGCCCTGAACGACCTGAACAATCTCGTGATCACC
AGGGACGAGAACGAGAAGCTGGAATATTACGAGAAGTTCCAGATCATCGA
GAACGTGTTCAAGCAGAAGAAGAAGCCCACCCTGAAGCAGATCGCCAAAG
AAATCCTCGTGAACGAAGAGGATATTAAGGGCTACAGAGTGACCAGCACC
GGCAAGCCCGAGTTCACCAACCTGAAGGTGTACCACGACATCAAGGACAT
TACCGCCCGGAAAGAGATTATTGAGAACGCCGAGCTGCTGGATCAGATTG
CCAAGATCCTGACCATCTACCAGAGCAGCGAGGACATCCAGGAAGAACTG
ACCAATCTGAACTCCGAGCTGACCCAGGAAGAGATCGAGCAGATCTCTAA
TCTGAAGGGCTATACCGGCACCCACAACCTGAGCCTGAAGGCCATCAACC
TGATCCTGGACGAGCTGTGGCACACCAACGACAACCAGATCGCTATCTTC
AACCGGCTGAAGCTGGTGCCCAAGAAGGTGGACCTGTCCCAGCAGAAAGA
GATCCCCACCACCCTGGTGGACGACTTCATCCTGAGCCCCGTCGTGAAGA
GAAGCTTCATCCAGAGCATCAAAGTGATCAACGCCATCATCAAGAAGTAC
GGCCTGCCCAACGACATCATTATCGAGCTGGCCCGCGAGAAGAACTCCAA
GGACGCCCAGAAAATGATCAACGAGATGCAGAAGCGGAACCGGCAGACCA
ACGAGCGGATCGAGGAAATCATCCGGACCACCGGCAAAGAGAACGCCAAG
TACCTGATCGAGAAGATCAAGCTGCACGACATGCAGGAAGGCAAGTGCCT
GTACAGCCTGGAAGCCATCCCTCTGGAAGATCTGCTGAACAACCCCTTCA
ACTATGAGGTGGACCACATCATCCCCAGAAGCGTGTCCTTCGACAACAGC
TTCAACAACAAGGTGCTCGTGAAGCAGGAAGAAgcCAGCAAGAAGGGCAA
CCGGACCCCATTCCAGTACCTGAGCAGCAGCGACAGCAAGATCAGCTACG
AAACCTTCAAGAAGCACATCCTGAATCTGGCCAAGGGCAAGGGCAGAATC
AGCAAGACCAAGAAAGAGTATCTGCTGGAAGAACGGGACATCAACAGGTT
CTCCGTGCAGAAAGACTTCATCAACCGGAACCTGGTGGATACCAGATACG
CCACCAGAGGCCTGATGAACCTGCTGCGGAGCTACTTCAGAGTGAACAAC
CTGGACGTGAAAGTGAAGTCCATCAATGGCGGCTTCACCAGCTTTCTGCG
GCGGAAGTGGAAGTTTAAGAAAGAGCGGAACAAGGGGTACAAGCACCACG
CCGAGGACGCCCTGATCATTGCCAACGCCGATTTCATCTTCAAAGAGTGG
AAGAAACTGGACAAGGCCAAAAAAGTGATGGAAAACCAGATGTTCGAGGA
AAAGCAGGCCGAGAGCATGCCCGAGATCGAAACCGAGCAGGAGTACAAAG
AGATCTTCATCACCCCCCACCAGATCAAGCACATTAAGGACTTCAAGGAC
TACAAGTACAGCCACCGGGTGGACAAGAAGCCTAATAGAGAGCTGATTAA
CGACACCCTGTACTCCACCCGGAAGGACGACAAGGGCAACACCCTGATCG
TGAACAATCTGAACGGCCTGTACGACAAGGACAATGACAAGCTGAAAAAG
CTGATCAACAAGAGCCCCGAAAAGCTGCTGATGTACCACCACGACCCCCA
GACCTACCAGAAACTGAAGCTGATTATGGAACAGTACGGCGACGAGAAGA
ATCCCCTGTACAAGTACTACGAGGAAACCGGGAACTACCTGACCAAGTAC
TCCAAAAAGGACAACGGCCCCGTGATCAAGAAGATTAAGTATTACGGCAA
CAAACTGAACGCCCATCTGGACATCACCGACGACTACCCCAACAGCAGAA
ACAAGGTCGTGAAGCTGTCCCTGAAGCCCTACAGATTCGACGTGTACCTG
GACAATGGCGTGTACAAGTTCGTGACCGTGAAGAATCTGGATGTGATCAA
AAAAGAAAACTACTACGAAGTGAATAGCAAGTGCTATGAGGAAGCTAAGA
AGCTGAAGAAGATCAGCAACCAGGCCGAGTTTATCGCCTCCTTCTACAAC
AACGATCTGATCAAGATCAACGGCGAGCTGTATAGAGTGATCGGCGTGAA
CAACGACCTGCTGAACCGGATCGAAGTGAACATGATCGACATCACCTACC
GCGAGTACCTGGAAAACATGAACGACAAGAGGCCCCCCAGGATCATTAAG
ACAATCGCCTCCAAGACCCAGAGCATTAAGAAGTACAGCACAGACATTCT
GGGCAACCTGTATGAAGTGAAATCTAAGAAGCACCCTCAGATCATCAAAA
AGGGCAAAAGGCCGGCGGCCACGAAAAAGGCCGGCCAGGCAAAAAAGAAA
AAGggatcCGATGCTAAGTCACTGACTGCCTGGTCCCGGACACTGGTGAC
CTTCAAGGATGTGTTTGTGGACTTCACCAGGGAGGAGTGGAAGCTGCTGG
ACACTGCTCAGCAGATCCTGTACAGAAATGTGATGCTGGAGAACTATAAG
AACCTGGTTTCCTTGGGTTATCAGCTTACTAAGCCAGATGTGATCCTCCG
GTTGGAGAAGGGAGAAGAGCCCTGGCTGGTGGAGAGAGAAATTCACCAAG
AGACCCATCCTGATTCAGAGACTGCATTTGAAATCAAATCATCAGTTCCG
AAAAAGAAACGCAAAGTTgctagCGAGGGCAGAGGAAGTCTTCTAACATG
CGGTGACGTGGAGGAGAATCCCGGCCCTATGACCGAGTACAAGCCCACGG
TGCGCCTCGCCACCCGCGACGACGTCCCCaGGGCCGTACGCACCCTCGCC
GCCGCGTTCGCCGACTACCCCGCCACGCGCCACACCGTCGATCCGGACCG
CCACATCGAGCGGGTCACCGAGCTGCAAGAACTCTTCCTCACGCGCGTCG
GGCTCGACATCGGCAAGGTGTGGGTCGCGGACGACGGCGCCGCGGTGGCG
GTCTGGACCACGCCGGAGAGCGTCGAAGCGGGGGCGGTGTTCGCCGAGAT
CGGCCCGCGCATGGCCGAGTTGAGCGGTTCCCGGCTGGCCGCGCAGCAAC
AGATGGAAGGCCTCCTGGCGCCGCACCGGCCCAAGGAGCCCGCGTGGTTC
CTGGCCACCGTCGGCGTGTCGCCCGACCACCAGGGCAAGGGTCTGGGCAG
CGCCGTCGTGCTCCCCGGAGTGGAGGCGGCCGAGCGCGCCGGGGTGCCCG
CCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTG
GTGCATGACCCGCAAGCCCGGTGCCTGACCAGcacactggcggcCGTTAC
TAGCTTCTGCAGCACGAccggTTGATAATAGATAACTTCGTATAGCATAC
ATTATACGAAGTTATGaattCGATATCAAGCTTATCGATAATCAACCTCT
GGATTACAAAATTTGTGAAAGATTGACTGGTATTCTTAACTATGTTGCTC
CTTTTACGCTATGTGGATACGCTGCTTTAATGCCTTTGTATCATGCTATT
GCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACC
TGTCAGCTCCTTTCCGGGACTTTCGCTTTCCCCCTCCCTATTGCCACGGC
GGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCT
TGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGC
TGCCGGCTCTGCGGCCTCTTCCGCGTCTTCGCCTTCGCCCTCAGACGAGT
CGGATCTCCCTTTGGGCCGCCTCCCCGCATCGATACCGTCGACCTCGAGA
CCTAGAAAAACATGGAGCAATCACAAGTAGCAATACAGCAGCTACCAATG
CTGATTGTGCCTGGCTAGAAGCACAAGAGGAGGAGGAGGTGGGTTTTCCA
GTCACACCTCAGGTACCTTTAAGACCAATGACTTACAAGGCAGCTGTAGA
TCTTAGCCACTTTTTAAAAGAAAAGGGGGGACTGGAAGGGCTAATTCACT
CCCAACGAAGACAAGATATCCTTGATCTGTGGATCTACCACACACAAGGC
TACTTCCCTGATTGGCAGAACTACACACCAGGGCCAGGGATCAGATATCC
ACTGACCTTTGGATGGTGCTACAAGCTAGTACCAGTTGAGCAAGAGAAGG
TAGAAGAAGCCAATGAAGGAGAGAACACCCGCTTGTTACACCCTGTGAGC
CTGCATGGGATGGATGACCCGGAGAGAGAAGTATTAGAGTGGAGGTTTGA
CAGCCGCCTAGCATTTCATCACATGGCCCGAGAGCTGCATCCGGACTGTA
CTGGGTCTCTCTGGTTAGACCAGATCTGAGCCTGGGAGCTCTCTGGCTAA
CTAGGGAACCCACTGCTTAAGCCTCAATAAAGCTTGCCTTGAGTGCTTCA
AGTAGTGTGTGCCCGTCTGTTGTGTGACTCTGGTAACTAGAGATCCCTCA
GACCCTTTTAGTCAGTGTGGAAAATCTCTAGCAGGGCCCGTTTAAACCCG
CTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAG
ACAATAGCAGGCATGCTGGGGATGCGGTGGGCTCTATGGCTTCTGAGGCG
GAAAGAACCAGCTGGGGCTCTAGGGGGTATCCCCACGCGCCCTGTAGCGG
CGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATT
AGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGTCTATTCTTTTGATTTATAAGGGA
TTTTGCCGATTTCGGCCTATTGGTTAAAAAATGAGCTGATTTAACAAAAA
TTTAACGCGAATTAATTCTGTGGAATGTGTGTCAGTTAGGGTGTGGAAAG
TCCCCAGGCTCCCCAGCAGGCAGAAGTATGCAAAGCATGCATCTCAATTA
GTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACT
CCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCT
CTGAGCTATTCCAGAAGTAGTGAGGAGGCTTTTTTGGAGGCCTAGGCTTT
TGCAAAAAGCTCCCGGGAGCTTGTATATCCATTTTCGGATCTGATCAGCA
CGTGTTGACAATTAATCATCGGCATAGTATATCGGCATAGTATAATACGA
CAAGGTGAGGAACTAAACCATGGCCAAGTTGACCAGTGCCGTTCCGGTGC
TCACCGCGCGCGACGTCGCCGGAGCGGTCGAGTTCTGGACCGACCGGCTC
GGGTTCTCCCGGGACTTCGTGGAGGACGACTTCGCCGGTGTGGTCCGGGA
CGACGTGACCCTGTTCATCAGCGCGGTCCAGGACCAGGTGGTGCCGGACA
ACACCCTGGCCTGGGTGTGGGTGCGCGGCCTGGACGAGCTGTACGCCGAG
TGGTCGGAGGTCGTGTCCACGAACTTCCGGGACGCCTCCGGGCCGGCCAT
GACCGAGATCGGCGAGCAGCCGTGGGGGCGGGAGTTCGCCCTGCGCGACC
CGGCCGGCAACTGCGTGCACTTCGTGGCCGAGGAGCAGGACTGACACGTG
CTACGAGATTTCGATTCCACCGCCGCCTTCTATGAAAGGTTGGGCTTCGG
AATCGTTTTCCGGGACGCCGGCTGGATGATCCTCCAGCGCGGGGATCTCA
TGCTGGAGTTCTTCGCCCACCCCAACTTGTTTATTGCAGCTTATAATGGT
TACAAATAAAGCAATAGCATCACAAATTTCACAAATAAAGCATTTTTTTC
ACTGCATTCTAGTTGTGGTTTGTCCAAACTCATCAATGTATCTTATCATG
TCTGTATACCGTCGACCTCTAGCTAGAGCTTGGCGTAATCATGGTCATAG
CTGTTTCCTGTGTGAAATTGTTATCCGCTCACAATTCCACACAACATACG
AGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTG
TCGTGCCAGCTGCATTAATGAATCGGCCAACGCGCGGGGAGAGGCGGTTT
GCGTATTGGGCGCTCTTCCGCTTCCTCGCTCACTGACTCGCTGCGCTCGG
TCGTTCGGCTGCGGCGAGCGGTATCAGCTCACTCAAAGGCGGTAATACGG
TTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAG
AGGTGGCGAAACCCGACAGGACTATAAAGATACCAGGCGTTTCCCCCTGG
AAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATC
GTCTTGAGTCCAACCCGGTAAGACACGACTTATCGCCACTGGCAGCAGCC
ACTGGTAACAGGATTAGCAGAGCGAGGTATGTAGGCGGTGCTACAGAGTT
CTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCT
TTTCTACGGGGTCTGACGCTCAGTGGAACGAAAACTCACGTTAAGGGATT
TTGGTCATGAGATTATCAAAAAGGATCTTCACCTAGATCCTTTTAAATTA
AAAATGAAGTTTTAAATCAATCTAAAGTATATATGAGTAAACTTGGTCTG
ACAGTTACCAATGCTTAATCAGTGAGGCACCTATCTCAGCGATCTGTCTA
TTTCGTTCATCCATAGTTGCCTGACTCCCCGTCGTGTAGATAACTACGAT
ACGGGAGGGCTTACCATCTGGCCCCAGTGCTGCAATGATACCGCGAGACC
CACGCTCACCGGCTCCAGATTTATCAGCAATAAACCAGCCAGCCGGAAGG
GCCGAGCGCAGAAGTGGTCCTGCAACTTTATCCGCCTCCATCCAGTCTAT
TAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTT
GGTATGGCTTCATTCAGCTCCGGTTCCCAACGATCAAGGCGAGTTACATG
ATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCA
CTGCATAATTCTCTTACTGTCATGCCATCCGTAAGATGCTTTTCTGTGAC
TGGTGAGTACTCAACCAAGTCATTCTGAGAATAGTGTATGCGGCGACCGA
GTTGCTCTTGCCCGGCGTCAATACGGGATAATACCGCGCCACATAGCAGA
ACTTTAAAAGTGCTCATCATTGGAAAACGTTCTTCGGGGCGAAAACTCTC
AAGGATCTTACCGCTGTTGAGATCCAGTTCGATGTAACCCACTCGTGCAC
CCAACTGATCTTCAGCATCTTTTACTTTCACCAGCGTTTCTGGGTGAGCA
AAAACAGGAAGGCAAAATGCCGCAAAAAAGGGAATAAGGGCGACACGGAA
ATGTTGAATACTCATACTCTTCCTTTTTCAATATTATTGAAGCATTTATC
AGGGTTATTGTCTCATGAGCGGATACATATTTGAATGTATTTAGAAAAAT
AAACAAATAGGGGTTCCGCGCACATTTCCCCGAAAAGTGCCACCTGAC

1.6.2 AAV S. aureus Cas9 KRAB-Based Repressor

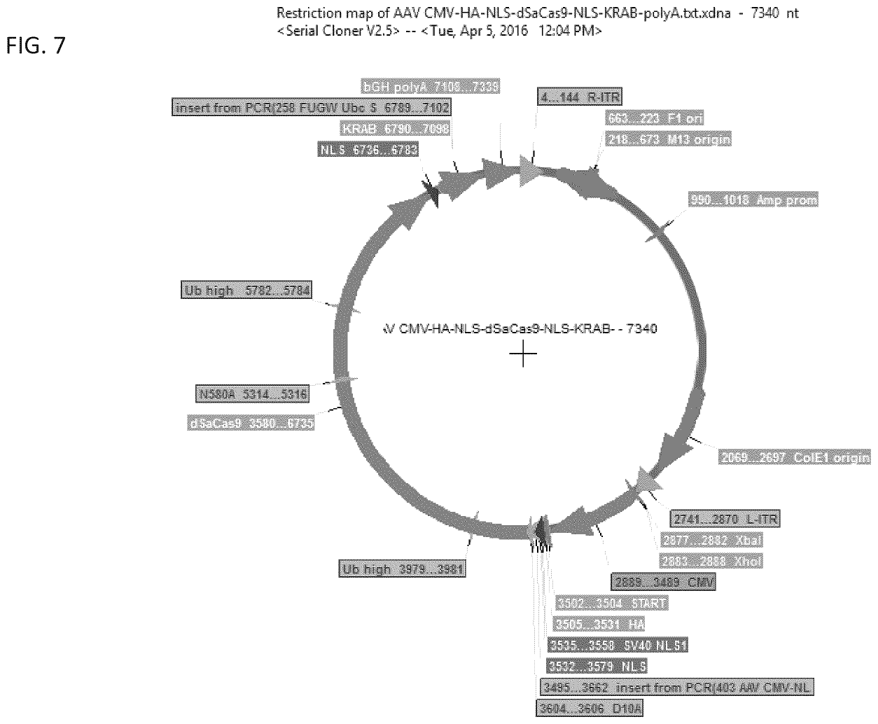

A restriction map of an AAV vector encoding S. aureus Cas9 KRAB-based repressor is shown in FIG. 7. SEQ ID NO: 3 provides the nucleic acid sequence of the AAV vector encoding S. aureus Cas9 KRAB-based repressor.

SEQ ID NO: 3
gcaggaacccctagtgatggagttggccactccctctctgcgcgctcgct
cgctcactgaggccgggcgaccaaaggtcgcccgacgcccgggctttgcc
cgggcggcctcagtgagcgagcgagcgcgcagctgcctgcaggggcgcct
gatgcggtattttctccttacgcatctgtgcggtatttcacaccgcatac
gtcaaagcaaccatagtacgcgccctgtagcggcgcattaagcgcggcgg
gtgtggtggttacgcgcagcgtgaccgctacacttgccagcgccctagcg
cccgctcctttcgctttcttcccttcctttctcgccacgttcgccggctt
tccccgtcaagctctaaatcgggggctccctttagggttccgatttagtg
ctttacggcacctcgaccccaaaaaacttgatttgggtgatggttcacgt
agtgggccatcgccctgatagacggtttttcgccctttgacgttggagtc
cacgttctttaatagtggactcttgttccaaactggaacaacactcaacc
ctatctcgggctattcttttgatttataagggattttgccgatttcggcc
tattggttaaaaaatgagctgatttaacaaaaatttaacgcgaattttaa
caaaatattaacgtttacaattttatggtgcactctcagtacaatctgct
ctgatgccgcatagttaagccagccccgacacccgccaacacccgctgac
gcgccctgacgggcttgtctgctcccggcatccgcttacagacaagctgt
gaccgtctccgggagctgcatgtgtcagaggttttcaccgtcatcaccga
aacgcgcgagacgaaagggcctcgtgatacgcctatttttataggttaat
gtcatgataataatggtttcttagacgtcaggtggcacttttcggggaaa
tgtgcgcggaacccctatttgtttatttttctaaatacattcaaatatgt
atccgctcatgagacaataaccctgataaatgcttcaataatattgaaaa
aggaagagtatgagtattcaacatttccgtgtcgcccttattcccttttt
tgcggcattttgccttcctgtttttgctcacccagaaacgctggtgaaag
taaaagatgctgaagatcagttgggtgcacgagtgggttacatcgaactg
gatctcaacagcggtaagatccttgagagttttcgccccgaagaacgttt
tccaatgatgagcacttttaaagttctgctatgtggcgcggtattatccc
gtattgacgccgggcaagagcaactcggtcgccgcatacactattctcag
aatgacttggttgagtactcaccagtcacagaaaagcatcttacggatgg
catgacagtaagagaattatgcagtgctgccataaccatgagtgataaca
ctgcggccaacttacttctgacaacgatcggaggaccgaaggagctaacc
gcttttttgcacaacatgggggatcatgtaactcgccttgatcgttggga
accggagctgaatgaagccataccaaacgacgagcgtgacaccacgatgc
ctgtagcaatggcaacaacgttgcgcaaactattaactggcgaactactt
actctagcttcccggcaacaattaatagactggatggaggcggataaagt
tgcaggaccacttctgcgctcggcccttccggctggctggtttattgctg
ataaatctggagccggtgagcgtggaagccgcggtatcattgcagcactg
gggccagatggtaagccctcccgtatcgtagttatctacacgacggggag
tcaggcaactatggatgaacgaaatagacagatcgctgagataggtgcct
cactgattaagcattggtaactgtcagaccaagtttactcatatatactt
tagattgatttaaaacttcatttttaatttaaaaggatctaggtgaagat
cctttttgataatctcatgaccaaaatcccttaacgtgagttttcgttcc
actgagcgtcagaccccgtagaaaagatcaaaggatcttcttgagatcct
ttttttctgcgcgtaatctgctgcttgcaaacaaaaaaaccaccgctacc
agcggtggtttgtttgccggatcaagagctaccaactctttttccgaagg
taactggcttcagcagagcgcagataccaaatactgtccttctagtgtag
ccgtagttaggccaccacttcaagaactctgtagcaccgcctacatacct
cgctctgctaatcctgttaccagtggctgctgccagtggcgataagtcgt
gtcttaccgggttggactcaagacgatagttaccggataaggcgcagcgg
tcgggctgaacggggggttcgtgcacacagcccagcttggagcgaacgac
ctacaccgaactgagatacctacagcgtgagctatgagaaagcgccacgc
ttcccgaagggagaaaggcggacaggtatccggtaagcggcagggtcgga
acaggagagcgcacgagggagcttccagggggaaacgcctggtatcttta
tagtcctgtcgggtttcgccacctctgacttgagcgtcgatttttgtgat
gctcgtcaggggggcggagcctatggaaaaacgccagcaacgcggccttt
ttacggttcctggccttttgctggccttttgctcacatgtcctgcaggca
gctgcgcgctcgctcgctcactgaggccgcccgggcgtcgggcgaccttt
ggtcgcccggcctcagtgagcgagcgagcgcgcagagagggagtggccaa
ctccatcactaggggttcctgcggcctctagactcgaggcgttgacattg
attattgactagttattaatagtaatcaattacggggtcattagttcata
gcccatatatggagttccgcgttacataacttacggtaaatggcccgcct
ggctgaccgcccaacgacccccgcccattgacgtcaataatgacgtatgt
tcccatagtaacgccaatagggactttccattgacgtcaatgggtggagt
atttacggtaaactgcccacttggcagtacatcaagtgtatcatatgcca
agtacgccccctattgacgtcaatgacggtaaatggcccgcctggcatta
tgcccagtacatgaccttatgggactttcctacttggcagtacatctacg
tattagtcatcgctattaccatggtgatgcggttttggcagtacatcaat
gggcgtggatagcggtttgactcacggggatttccaagtctccaccccat
tgacgtcaatgggagtttgttttggcaccaaaatcaacgggactttccaa
aatgtcgtaacaactccgccccattgacgcaaatgggcggtaggcgtgta
cggtgggaggtctatataagcagagctctctggctaactaccggtgccac
catgtacccatacgatgttccagattacgctGCCCCAAAGAAGAAGCGGA
AGGTCGGTATCCACGGAGTCCCAGCAGCCAAGCGGAACTACATCCTGGGC
CTGGCCATCGGCATCACCAGCGTGGGCTACGGCATCATCGACTACGAGAC
ACGGGACGTGatcgATGCCGGCGTGCGGCTGTTCAAAGAGGCCAACGTGG
AAAACAACGAGGGCAGGCGGAGCAAGAGAGGCGCCAGAAGGCTGAAGCGG
CGGAGGCGGCATAGAATCCAGAGAGTGAAGAAGCTGCTGTTCGACTACAA
CCTGCTGACCGACCACAGCGAGCTGAGCGGCATCAACCCCTACGAGGCCA
GAGTGAAGGGCCTGAGCCAGAAGCTGAGCGAGGAAGAGTTCTCTGCCGCC
CTGCTGCACCTGGCCAAGAGAAGAGGCGTGCACAACGTGAACGAGGTGGA
AGAGGACACCGGCAACGAGCTGTCCACCAAAGAGCAGATCAGCCGGAACA
GCAAGGCCCTGGAAGAGAAATACGTGGCCGAACTGCAGCTGGAACGGCTG
AAGAAAGACGGCGAAGTGCGGGGCAGCATCAACAGATTCAAGACCAGCGA
CTACGTGAAAGAAGCCAAACAGCTGCTGAAGGTGCAGAAGGCCTACCACC
AGCTGGACCAGAGCTTCATCGACACCTACATCGACCTGCTGGAAACCCGG
CGGACCTACTATGAGGGACCTGGCGAGGGCAGCCCCTTCGGCTGGAAGGA
CATCAAAGAATGGTACGAGATGCTGATGGGCCACTGCACCTACTTCCCCG
AGGAACTGCGGAGCGTGAAGTACGCCTACAACGCCGACCTGTACAACGCC
CTGAACGACCTGAACAATCTCGTGATCACCAGGGACGAGAACGAGAAGCT
GGAATATTACGAGAAGTTCCAGATCATCGAGAACGTGTTCAAGCAGAAGA
AGAAGCCCACCCTGAAGCAGATCGCCAAAGAAATCCTCGTGAACGAAGAG
GATATTAAGGGCTACAGAGTGACCAGCACCGGCAAGCCCGAGTTCACCAA
CCTGAAGGTGTACCACGACATCAAGGACATTACCGCCCGGAAAGAGATTA
TTGAGAACGCCGAGCTGCTGGATCAGATTGCCAAGATCCTGACCATCTAC
CAGAGCAGCGAGGACATCCAGGAAGAACTGACCAATCTGAACTCCGAGCT
GACCCAGGAAGAGATCGAGCAGATCTCTAATCTGAAGGGCTATACCGGCA
CCCACAACCTGAGCCTGAAGGCCATCAACCTGATCCTGGACGAGCTGTGG
CACACCAACGACAACCAGATCGCTATCTTCAACCGGCTGAAGCTGGTGCC
CAAGAAGGTGGACCTGTCCCAGCAGAAAGAGATCCCCACCACCCTGGTGG
ACGACTTCATCCTGAGCCCCGTCGTGAAGAGAAGCTTCATCCAGAGCATC
AAAGTGATCAACGCCATCATCAAGAAGTACGGCCTGCCCAACGACATCAT
TATCGAGCTGGCCCGCGAGAAGAACTCCAAGGACGCCCAGAAAATGATCA
ACGAGATGCAGAAGCGGAACCGGCAGACCAACGAGCGGATCGAGGAAATC
ATCCGGACCACCGGCAAAGAGAACGCCAAGTACCTGATCGAGAAGATCAA
GCTGCACGACATGCAGGAAGGCAAGTGCCTGTACAGCCTGGAAGCCATCC
CTCTGGAAGATCTGCTGAACAACCCCTTCAACTATGAGGTGGACCACATC
ATCCCCAGAAGCGTGTCCTTCGACAACAGCTTCAACAACAAGGTGCTCGT
GAAGCAGGAAGAAgcCAGCAAGAAGGGCAACCGGACCCCATTCCAGTACC
TGAGCAGCAGCGACAGCAAGATCAGCTACGAAACCTTCAAGAAGCACATC
CTGAATCTGGCCAAGGGCAAGGGCAGAATCAGCAAGACCAAGAAAGAGTA
TCTGCTGGAAGAACGGGACATCAACAGGTTCTCCGTGCAGAAAGACTTCA
TCAACCGGAACCTGGTGGATACCAGATACGCCACCAGAGGCCTGATGAAC
CTGCTGCGGAGCTACTTCAGAGTGAACAACCTGGACGTGAAAGTGAAGTC
CATCAATGGCGGCTTCACCAGCTTTCTGCGGCGGAAGTGGAAGTTTAAGA
AAGAGCGGAACAAGGGGTACAAGCACCACGCCGAGGACGCCCTGATCATT
GCCAACGCCGATTTCATCTTCAAAGAGTGGAAGAAACTGGACAAGGCCAA
AAAAGTGATGGAAAACCAGATGTTCGAGGAAAAGCAGGCCGAGAGCATGC
CCGAGATCGAAACCGAGCAGGAGTACAAAGAGATCTTCATCACCCCCCAC
CAGATCAAGCACATTAAGGACTTCAAGGACTACAAGTACAGCCACCGGGT
GGACAAGAAGCCTAATAGAGAGCTGATTAACGACACCCTGTACTCCACCC
GGAAGGACGACAAGGGCAACACCCTGATCGTGAACAATCTGAACGGCCTG
TACGACAAGGACAATGACAAGCTGAAAAAGCTGATCAACAAGAGCCCCGA
AAAGCTGCTGATGTACCACCACGACCCCCAGACCTACCAGAAACTGAAGC
TGATTATGGAACAGTACGGCGACGAGAAGAATCCCCTGTACAAGTACTAC
GAGGAAACCGGGAACTACCTGACCAAGTACTCCAAAAAGGACAACGGCCC
CGTGATCAAGAAGATTAAGTATTACGGCAACAAACTGAACGCCCATCTGG
ACATCACCGACGACTACCCCAACAGCAGAAACAAGGTCGTGAAGCTGTCC
CTGAAGCCCTACAGATTCGACGTGTACCTGGACAATGGCGTGTACAAGTT
CGTGACCGTGAAGAATCTGGATGTGATCAAAAAAGAAAACTACTACGAAG
TGAATAGCAAGTGCTATGAGGAAGCTAAGAAGCTGAAGAAGATCAGCAAC
CAGGCCGAGTTTATCGCCTCCTTCTACAACAACGATCTGATCAAGATCAA
CGGCGAGCTGTATAGAGTGATCGGCGTGAACAACGACCTGCTGAACCGGA
TCGAAGTGAACATGATCGACATCACCTACCGCGAGTACCTGGAAAACATG
AACGACAAGAGGCCCCCCAGGATCATTAAGACAATCGCCTCCAAGACCCA
GAGCATTAAGAAGTACAGCACAGACATTCTGGGCAACCTGTATGAAGTGA
AATCTAAGAAGCACCCTCAGATCATCAAAAAGGGCAAAAGGCCGGCGGCC
ACGAAAAAGGCCGGCCAGGCAAAAAAGAAAAAGggatcCGATGCTAAGTC
ACTGACTGCCTGGTCCCGGACACTGGTGACCTTCAAGGATGTGTTTGTGG
ACTTCACCAGGGAGGAGTGGAAGCTGCTGGACACTGCTCAGCAGATCCTG
TACAGAAATGTGATGCTGGAGAACTATAAGAACCTGGTTTCCTTGGGTTA
TCAGCTTACTAAGCCAGATGTGATCCTCCGGTTGGAGAAGGGAGAAGAGC
CCTGGCTGGTGGAGAGAGAAATTCACCAAGAGACCCATCCTGATTCAGAG
ACTGCATTTGAAATCAAATCATCAGTTCCGAAAAAGAAACGCAAAGttta
aGaattcctagagctcgctgatcagcctcgactgtgccttctagttgcca
gccatctgttgtttgcccctcccccgtgccttccttgaccctggaaggtg
ccactcccactgtcctttcctaataaaatgaggaaattgcatcgcattgt
ctgagtaggtgtcattctattctggggggtggggtggggcaggacagcaa
gggggaggattgggaagagaatagcaggcatgctggggag

1.6.3 AAV S. aureus Cas9 U6-gRNA Vector with GFP-Kan Stuffer

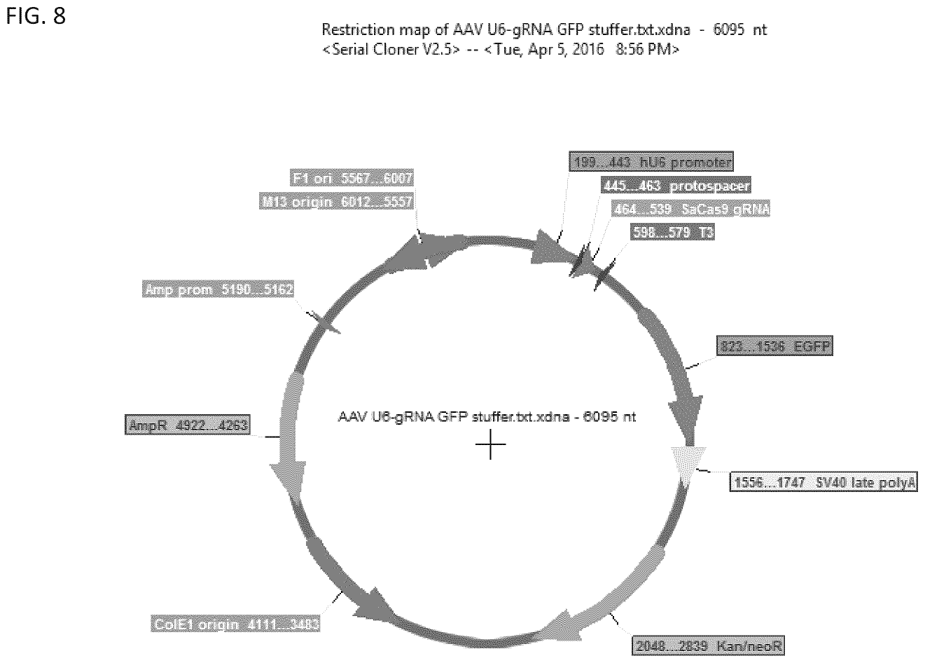

A restriction map of an AAV vector encoding S. aureus Cas9 U6-gRNA is shown in FIG. 8. SEQ ID NO: 4 provides the nucleic acid sequence of the AAV vector encoding S. aureus Cas9 U6-gRNA (with sample protospacer gRNA sequence).

SEQ ID NO: 4
ggggggggggggggggggttggccactccctctctgcgcgctcgctcgct
cactgaggccgggcgaccaaaggtcgcccgacgcccgggctttgcccggg
cggcctcagtgagcgagcgagcgcgcagagagggagtggccaactccatc
actaggggttcctagatctgaattcggtacCagatctaggaaCCTAGGgc
ctatttcccatgattccttcatatttgcatatacgatacaaggctgttag
agagataattggaattaatttgactgtaaacacaaagatattagtacaaa
atacgtgacgtagaaagtaataatttcttgggtagtttgcagttttaaaa
ttatgttttaaaatggactatcatatgcttaccgtaacttgaaagtattt
cgatttcttggctttatatatcttgTGGAAAGGACGAAACACCgagcgcg
ccccgcctagcccgttttagtactctggaaacagaatctactaaaacaag
gcaaaatgccgtgtttatctcgtcaacttgttggcgagatttttttGCGG
CCGCCCgcggtggagctccagcttttgttccctttagtgagggttaatTc
tagaggatccggtactcgaggaactgaaaaaccagaaagttaactggtaa
gtttagtctttttgtcttttatttcaggtcccggatccggtggtggtgca
aatcaaagaactgctcctcagtggatgttgcctttacttctaggcctgta
cggaagtgttacttctgctctaaaagctgcggaattgtacccgcggcccg
ggatccaccggtcgccaccatggtgagcaagggcgaggagctgttcaccg
gggtggtgcccatcctggtcgagctggacggcgacgtaaacggccacaag
ttcagcgtgtccggcgagggcgagggcgatgccacctacggcaagctgac
cctgaagttcatctgcaccaccggcaagctgcccgtgccctggcccaccc
tcgtgaccaccctgacctacggcgtgcagtgcttcagccgctaccccgac
cacatgaagcagcacgacttcttcaagtccgccatgcccgaaggctacgt
ccaggagcgcaccatcttcttcaaggacgacggcaactacaagacccgcg
ccgaggtgaagttcgagggcgacaccctggtgaaccgcatcgagctgaag
ggcatcgacttcaaggaggacggcaacatcctggggcacaagctggagta
caactacaacagccacaacgtctatatcatggccgacaagcagaagaacg
gcatcaaggtgaacttcaagatccgccacaacatcgaggacggcagcgtg
cagctcgccgaccactaccagcagaacacccccatcggcgacggccccgt
gctgctgcccgacaaccactacctgagcacccagtccgccctgagcaaag
accccaacgagaagcgcgatcacatggtcctgctggagttcgtgaccgcc
gccgggatcactctcggcatggacgagctgtacaagtaaagcggccgcgg
ggatccagacatgataagatacattgatgagtttggacaaaccacaacta
gaatgcagtgaaaaaaatgctttatttgtgaaatttgtgatgctattgct
ttatttgtaaccattataagctgcaataaacaagttaacaacaacaattg
cattcattttatgtttcaggttcagggggaggtgtgggaggttttttagt
cgacctcgagcagtgtggttttgcaagaggaagcaaaaagcctctccacc
caggcctggaatgtttccacccaagtcgaaggcagtgtggttttgcaaga
ggaagcaaaaagcctctccacccaggcctggaatgtttccacccaatgtc
gagcaaccccgcccagcgtcttgtcattggcgaattcgaacacgcagatg
cagtcggggcggcgcggtcccaggtccacttcgcatattaaggtgacgcg
tgtggcctcgaacaccgagcgaccctgcagccaatatgggatcggccatt
gaacaagatggattgcacgcaggttctccggccgcttgggtggagaggct
attcggctatgactgggcacaacagacaatcggctgctctgatgccgccg
tgttccggctgtcagcgcaggggcgcccggttctttttgtcaagaccgac
ctgtccggtgccctgaatgaactgcaggacgaggcagcgcggctatcgtg
gctggccacgacgggcgttccttgcgcagctgtgctcgacgttgtcactg
aagcgggaagggactggctgctattgggcgaagtgccggggcaggatctc
ctgtcatctcaccttgctcctgccgagaaagtatccatcatggctgatgc
aatgcggcggctgcatacgcttgatccggctacctgcccattcgaccacc
aagcgaaacatcgcatcgagcgagcacgtactcggatggaagccggtctt
gtcgatcaggatgatctggacgaagagcatcaggggctcgcgccagccga
actgttcgccaggctcaaggcgcgcatgcccgacggcgaggatctcgtcg
tgacccatggcgatgcctgcttgccgaatatcatggtggaaaatggccgc
ttttctggattcatcgactgtggccggctgggtgtggcggaccgctatca
ggacatagcgttggctacccgtgatattgctgaagagcttggcggcgaat
gggctgaccgcttcctcgtgctttacggtatcgccgctcccgattcgcag
cgcatcgccttctatcgccttcttgacgagttcttctgaggggatccgtc
gactagagctcgctgatcagcctcgactgtgccttctagttgccagccat
ctgttgtttgcccctcccccgtgccttccttgaccctggaaggtgccact
cccactgtcctttcctaataaaatgaggaaattgcatcgcattgtctgag
taggtgtcattctattctggggggtggggtggggcaggacagcaaggggg
aggattgggaagacaatagcaggcatgctggggagagatctaggaacccc
tagtgatggagttggccactccctctctgcgcgctcgctcgctcactgag
gccgcccgggcaaagcccgggcgtcgggcgacctttggtcgcccggcctc
agtgagcgagcgagcgcgcagagagggagtggccaacccccccccccccc
cccctgcagcccagctgcattaatgaatcggccaacgcgcggggagaggc
ggtttgcgtattgggcgctcttccgcttcctcgctcactgactcgctgcg
ctcggtcgttcggctgcggcgagcggtatcagctcactcaaaggcggtaa
tacggttatccacagaatcaggggataacgcaggaaagaacatgtgagca
aaaggccagcaaaaggccaggaaccgtaaaaaggccgcgttgctggcgtt
tttccataggctccgcccccctgacgagcatcacaaaaatcgacgctcaa
gtcagaggtggcgaaacccgacaggactataaagataccaggcgtttccc
cctggaagctccctcgtgcgctctcctgttccgaccctgccgcttaccgg
atacctgtccgcctttctcccttcgggaagcgtggcgctttctcaatgct
cacgctgtaggtatctcagttcggtgtaggtcgttcgctccaagctgggc
tgtgtgcacgaaccccccgttcagcccgaccgctgcgccttatccggtaa
ctatcgtcttgagtccaacccggtaagacacgacttatcgccactggcag
cagccactggtaacaggattagcagagcgaggtatgtaggcggtgctaca
gagttcttgaagtggtggcctaactacggctacactagaaggacagtatt
tggtatctgcgctctgctgaagccagttaccttcggaaaaagagttggta
gctcttgatccggcaaacaaaccaccgctggtagcggtggtttttttgtt
tgcaagcagcagattacgcgcagaaaaaaaggatctcaagaagatccttt
gatcttttctacggggtctgacgctcagtggaacgaaaactcacgttaag
ggattttggtcatgagattatcaaaaaggatcttcacctagatcctttta
aattaaaaatgaagttttaaatcaatctaaagtatatatgagtaaacttg
gtctgacagttaccaatgcttaatcagtgaggcacctatctcagcgatct
gtctatttcgttcatccatagttgcctgactccccgtcgtgtagataact
acgatacgggagggcttaccatctggccccagtgctgcaatgataccgcg
agacccacgctcaccggctccagatttatcagcaataaaccagccagccg
gaagggccgagcgcagaagtggtcctgcaactttatccgcctccatccag
tctattaattgttgccgggaagctagagtaagtagttcgccagttaatag
tttgcgcaacgttgttgccattgctacaggcatcgtggtgtcacgctcgt
cgtttggtatggcttcattcagctccggttcccaacgatcaaggcgagtt
acatgatcccccatgttgtgcaaaaaagcggttagctccttcggtcctcc
gatcgttgtcagaagtaagttggccgcagtgttatcactcatggttatgg
cagcactgcataattctcttactgtcatgccatccgtaagatgcttttct
gtgactggtgagtactcaaccaagtcattctgagaatagtgtatgcggcg
accgagttgctcttgcccggcgtcaatacgggataataccgcgccacata
gcagaactttaaaagtgctcatcattggaaaacgttcttcggggcgaaaa
ctctcaaggatcttaccgctgttgagatccagttcgatgtaacccactcg
tgcacccaactgatcttcagcatcttttactttcaccagcgtttctgggt
gagcaaaaacaggaaggcaaaatgccgcaaaaaagggaataagggcgaca
cggaaatgttgaatactcatactcttcctttttcaatattattgaagcat
ttatcagggttattgtctcatgagcggatacatatttgaatgtatttaga
aaaataaacaaataggggttccgcgcacatttccccgaaaagtgccacct
gacgtctaagaaaccattattatcatgacattaacctataaaaataggcg
tatcacgaggccctttcgtctcgcgcgtttcggtgatgacggtgaaaacc
tctgacacatgcagctcccggagacggtcacagcttgtctgtaagcggat
gccgggagcagacaagcccgtcagggcgcgtcagcgggtgttggcgggtg
tcggggctggcttaactatgcggcatcagagcagattgtactgagagtgc
accatatgcggtgtgaaataccgcacagatgcgtaaggagaaaataccgc
atcaggaaattgtaaacgttaatattttgttaaaattcgcgttaaatttt
tgttaaatcagctcattttttaaccaataggccgaaatcggcaaaatccc
ttataaatcaaaagaatagaccgagatagggttgagtgttgttccagttt
ggaacaagagtccactattaaagaacgtggactccaacgtcaaagggcga
aaaaccgtctatcagggcgatggcccactacgtgaaccatcaccctaatc
aagttttttggggtcgaggtgccgtaaagcactaaatcggaaccctaaag
ggagcccccgatttagagcttgacggggaaagccggcgaacgtggcgaga
aaggaagggaagaaagcgaaaggagcgggcgctagggcgctggcaagtgt
agcggtcacgctgcgcgtaaccaccacacccgccgcgcttaatgcgccgc
tacagggcgcgtcgcgccattcgccattcaggctacgcaactgttgggaa
gggcgatcggtgcgggcctcttcgctattacgccagctggctgca

1.6.5 AAV S. aureus Cas9 U6-gRNA Vector with GFP-Kan Stuffer

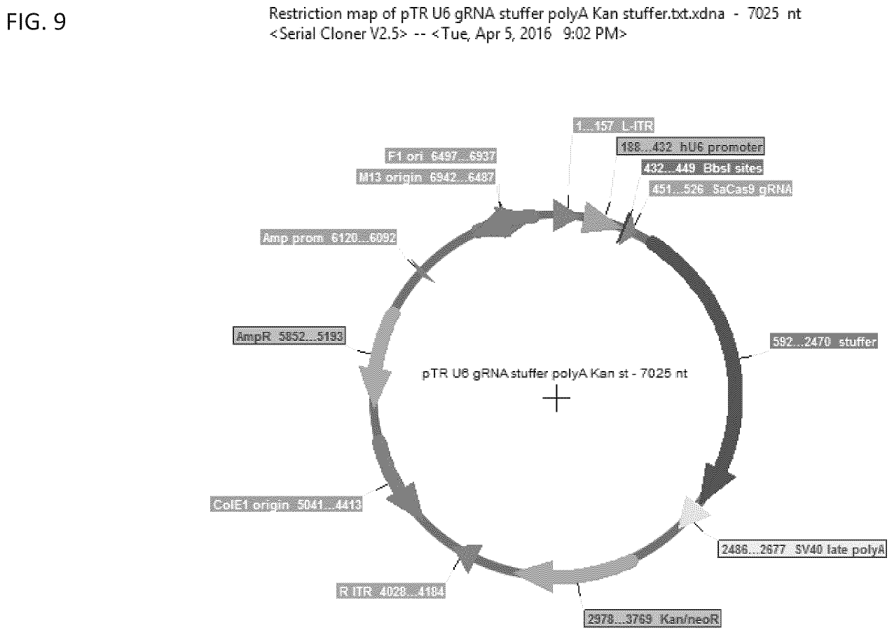

A restriction map of an AAV vector encoding S. aureus Cas9 U6-gRNA is shown in FIG. 9. SEQ ID NO: 5 provides the nucleic acid sequence of the AAV vector encoding S. aureus Cas9 U6-gRNA (Protospacer is cloned into the BbsI sites).

SEQ ID NO: 5
ggggggggggggggggggttggccactccctctctgcgcgctcgctcgct
cactgaggccgggcgaccaaaggtcgcccgacgcccgggctttgcccggg
cggcctcagtgagcgagcgagcgcgcagagagggagtggccaactccatc
actaggggttcctagatctgaattcggtaccaagctTgcctatttcccat
gattccttcatatttgcatatacgatacaaggctgttagagagataattg
gaattaatttgactgtaaacacaaagatattagtacaaaatacgtgacgt
agaaagtaataatttcttgggtagtttgcagttttaaaattatgttttaa
aatggactatcatatgcttaccgtaacttgaaagtatttcgatttcttgg
ctttatatatcttgTGGAAAGGACGAAACACCgggtcttcgagaagacct
gttttagtactctggaaacagaatctactaaaacaaggcaaaatgccgtg
tttatctcgtcaacttgttggcgagatttttttGCGGCCGCCCgcggtgg
agctccagcttttgttccctttagtgagggttaatTctagAgagacgtac
aaaaaagagcaagaagctaaaaaagatttaaaaattatttttagcgcagt
taatggaacaggaactaaatttaccccaaaaatattacgtgaatcaggat
ataacgttattgaggttgaagagcatgcatttgaagatgaaacatttaaa
aatgttgtaaatccaaatccagaatttgatcctgcatgaaaaataccgct
tgaatatggtattaaacatgatgcagatattattattatgaatgacccag
atgctgacagatttggaatggcaataaaacatgatggtcattttgtaaga
ttagatggaaatcaaacaggaccaattttaattgattgaaaattatcaaa
tctaaaacgcttaaatagcattccaaaaaatccggctctatattcaagtt
ttgtaacaagtgatttgggtgatagaatcgctcatgaaaaatatggagtt
aatattgtaaaaactttaactggatttaaatgaatgggtagagaaattgc
taaagaagaagataacggattaaattttgtttttgcttatgaagaaagtt
atggatatgtaattgatgactcagctagagataaagatggaatacaagct
tctatattaatagcagaggctgcttgattttataaaaaacaaaataaaac
attagtagactatttagaagatttatttaaagaaatgggtgcatattaca
ctttcactttaaacttgaattttaaaccagaagaaaagaaattaaaaatt
gaaccattaatgaaatcattgagagcaacacccttaactcaaattgctgg
acttaaagttgttaatgttgaagactacatcgatggaatgtataatatgc
caggacaagacttactaaaattttatttagaagataagtcatgatttgct
gttcgcccaagtggaactgaacctaaactaaaaatttattttataggtgt
tggtgaatctgttcaaaacgctaaagttaaagtagacgaaattattaaag
aattaaaattaaaaatgaatatataggagaaaaaatgaaactaaacaaat
atatagatcacacattattaaaacaagatgctacgaaagctgaaattaaa
caattatgtgatgaagcaattgaatttgattttgcaacagtttgtgttaa
ttcatattgaacaagctattgtaaagaattattaaaaggcacaaatgtag
gaataacaaatgttgtaggttttcctctaggtgcatgcacaacagctaca
aaagcattcgaagtttctgaagcaattaaagatggtgcaacagaaattga
tatggtattaaatattggtgcattaaaagacaaaaattatgaattagttt
tagaagacatgaaagctgtaaaaaaagcagctggatcacatgttgttaaa
tgtattatggaaaattgtttattaacaaaagaagaaatcatgaaagcttg
tgaaatagctgttgaagctggattagaatttgttaaaacatcaacaggat
tttcaaaatcaggtgcaacatttgaagatgttaaactaatgaagtcagtt
gttaaagacaatgctttagttaaagcagctggtggagttagaacatttga
agatgctcaaaaaatgattgaagcaggagctgaccgcttaggaacaagtg
gtggagtagctattattaaaggtgaagaaaacaacgcgagttactaaaac
tagcgtttttttattttgctcatttttattaaaagtttgcaaaaaggaac
ataaaaattctaattattgatactaaagttattaaaaagaagattttggt
tgattttataaaggtcatagaatataatattttagcatgtgtattttgtg
tgctcatttacaaccgtctcGCggccgcggggatccagacatgataagat
acattgatgagtttggacaaaccacaactagaatgcagtgaaaaaaatgc
tttatttgtgaaatttgtgatgctattgctttatttgtaaccattataag
ctgcaataaacaagttaacaacaacaattgcattcattttatgtttcagg
ttcagggggaggtgtgggaggttttttagtcgacctcgagcagtgtggtt
ttgcaagaggaagcaaaaagcctctccacccaggcctggaatgtttccac
ccaagtcgaaggcagtgtggttttgcaagaggaagcaaaaagcctctcca
cccaggcctggaatgtttccacccaatgtcgagcaaccccgcccagcgtc
ttgtcattggcgaattcgaacacgcagatgcagtcggggcggcgcggtcc
caggtccacttcgcatattaaggtgacgcgtgtggcctcgaacaccgagc
gaccctgcagccaatatgggatcggccattgaacaagatggattgcacgc
aggttctccggccgcttgggtggagaggctattcggctatgactgggcac
aacagacaatcggctgctctgatgccgccgtgttccggctgtcagcgcag
gggcgcccggttctttttgtcaagaccgacctgtccggtgccctgaatga
actgcaggacgaggcagcgcggctatcgtggctggccacgacgggcgttc
cttgcgcagctgtgctcgacgttgtcactgaagcgggaagggactggctg
ctattgggcgaagtgccggggcaggatctcctgtcatctcaccttgctcc
tgccgagaaagtatccatcatggctgatgcaatgcggcggctgcatacgc
ttgatccggctacctgcccattcgaccaccaagcgaaacatcgcatcgag
cgagcacgtactcggatggaagccggtcttgtcgatcaggatgatctgga
cgaagagcatcaggggctcgcgccagccgaactgttcgccaggctcaagg
cgcgcatgcccgacggcgaggatctcgtcgtgacccatggcgatgcctgc
ttgccgaatatcatggtggaaaatggccgcttttctggattcatcgactg
tggccggctgggtgtggcggaccgctatcaggacatagcgttggctaccc
gtgatattgctgaagagcttggcggcgaatgggctgaccgcttcctcgtg
ctttacggtatcgccgctcccgattcgcagcgcatcgccttctatcgcct
tcttgacgagttcttctgaggggatccgtcgactagagctcgctgatcag
cctcgactgtgccttctagttgccagccatctgttgtttgcccctccccc
gtgccttccttgaccctggaaggtgccactcccactgtcctttcctaata
aaatgaggaaattgcatcgcattgtctgagtaggtgtcattctattctgg
ggggtggggtggggcaggacagcaagggggaggattgggaagacaatagc
aggcatgctggggagagatctaggaacccctagtgatggagttggccact
ccctctctgcgcgctcgctcgctcactgaggccgcccgggcaaagcccgg
gcgtcgggcgacctttggtcgcccggcctcagtgagcgagcgagcgcgca
gagagggagtggccaacccccccccccccccccctgcagcccagctgcat
taatgaatcggccaacgcgcggggagaggcggtttgcgtattgggcgctc
ttccgcttcctcgctcactgactcgctgcgctcggtcgttcggctgcggc
gagcggtatcagctcactcaaaggcggtaatacggttatccacagaatca
ggggataacgcaggaaagaacatgtgagcaaaaggccagcaaaaggccag
gaaccgtaaaaaggccgcgttgctggcgtttttccataggctccgccccc
ctgacgagcatcacaaaaatcgacgctcaagtcagaggtggcgaaacccg
acaggactataaagataccaggcgtttccccctggaagctccctcgtgcg
ctctcctgttccgaccctgccgcttaccggatacctgtccgcctttctcc
cttcgggaagcgtggcgctttctcaatgctcacgctgtaggtatctcagt
tcggtgtaggtcgttcgctccaagctgggctgtgtgcacgaaccccccgt
tcagcccgaccgctgcgccttatccggtaactatcgtcttgagtccaacc
cggtaagacacgacttatcgccactggcagcagccactggtaacaggatt
agcagagcgaggtatgtaggcggtgctacagagttcttgaagtggtggcc
taactacggctacactagaaggacagtatttggtatctgcgctctgctga
agccagttaccttcggaaaaagagttggtagctcttgatccggcaaacaa
accaccgctggtagcggtggtttttttgtttgcaagcagcagattacgcg
cagaaaaaaaggatctcaagaagatcctttgatcttttctacggggtctg
acgctcagtggaacgaaaactcacgttaagggattttggtcatgagatta
tcaaaaaggatcttcacctagatccttttaaattaaaaatgaagttttaa
atcaatctaaagtatatatgagtaaacttggtctgacagttaccaatgct
taatcagtgaggcacctatctcagcgatctgtctatttcgttcatccata
gttgcctgactccccgtcgtgtagataactacgatacgggagggcttacc
atctggccccagtgctgcaatgataccgcgagacccacgctcaccggctc
cagatttatcagcaataaaccagccagccggaagggccgagcgcagaagt
ggtcctgcaactttatccgcctccatccagtctattaattgttgccggga
agctagagtaagtagttcgccagttaatagtttgcgcaacgttgttgcca
ttgctacaggcatcgtggtgtcacgctcgtcgtttggtatggcttcattc
agctccggttcccaacgatcaaggcgagttacatgatcccccatgttgtg
caaaaaagcggttagctccttcggtcctccgatcgttgtcagaagtaagt
tggccgcagtgttatcactcatggttatggcagcactgcataattctctt
actgtcatgccatccgtaagatgcttttctgtgactggtgagtactcaac
caagtcattctgagaatagtgtatgcggcgaccgagttgctcttgcccgg
cgtcaatacgggataataccgcgccacatagcagaactttaaaagtgctc
atcattggaaaacgttcttcggggcgaaaactctcaaggatcttaccgct
gttgagatccagttcgatgtaacccactcgtgcacccaactgatcttcag
catcttttactttcaccagcgtttctgggtgagcaaaaacaggaaggcaa
aatgccgcaaaaaagggaataagggcgacacggaaatgttgaatactcat
actcttcctttttcaatattattgaagcatttatcagggttattgtctca
tgagcggatacatatttgaatgtatttagaaaaataaacaaataggggtt
ccgcgcacatttccccgaaaagtgccacctgacgtctaagaaaccattat
tatcatgacattaacctataaaaataggcgtatcacgaggccctttcgtc
tcgcgcgtttcggtgatgacggtgaaaacctctgacacatgcagctcccg
gagacggtcacagcttgtctgtaagcggatgccgggagcagacaagcccg
tcagggcgcgtcagcgggtgttggcgggtgtcggggctggcttaactatg
cggcatcagagcagattgtactgagagtgcaccatatgcggtgtgaaata
ccgcacagatgcgtaaggagaaaataccgcatcaggaaattgtaaacgtt
aatattttgttaaaattcgcgttaaatttttgttaaatcagctcattttt
taaccaataggccgaaatcggcaaaatcccttataaatcaaaagaataga
ccgagatagggttgagtgttgttccagtttggaacaagagtccactatta
aagaacgtggactccaacgtcaaagggcgaaaaaccgtctatcagggcga
tggcccactacgtgaaccatcaccctaatcaagttttttggggtcgaggt
gccgtaaagcactaaatcggaaccctaaagggagcccccgatttagagct
tgacggggaaagccggcgaacgtggcgagaaaggaagggaagaaagcgaa
aggagcgggcgctagggcgctggcaagtgtagcggtcacgctgcgcgtaa
ccaccacacccgccgcgcttaatgcgccgctacagggcgcgtcgcgccat
tcgccattcaggctacgcaactgttgggaagggcgatcggtgcgggcctc
ttcgctattacgccagctggctgca

1.6.6 Protospacer Sequences for gRNAs

TABLE 4
CAG Luciferase gRNAs

| SEQ ID NO | Description | Sequence |
|---|---|---|
| 6 | SaCr1 | GTCATTATTGACGTCAATGGGC |
| 7 | SaCr2 | gtgctcagcaactcggggag |
| 8 | SaCr3 | ctcggggaggggggtgcagg |
| 9 | SaCr4 | ACTTTCCATTGACGTCAATGGG |
| 10 | SaCr5 | CTTCGGGGGGGACGGGGCAGGG |
| 11 | SaCr6 | cttcgccccgcgcccgctaga |
| 12 | SaCr7 | tcggggaggggggtgcagg |
| 13 | SaCr8 | tgctcagcaactcggggag |
| 14 | SaCr9 | gcggggggtggcggcaggt |

TABLE 5
Mouse Acvr2b gRNAs

| SEQ ID NO | Description | Sequence |
|---|---|---|
| 15 | SaCr1 | gctcctctgggacccctga |
| 16 | SaCr2 | tgctatggagcccacgcta |
| 17 | SaCr3 | ggcgcgctctccgagctgg |
| 18 | SaCr4 | agcgcgccccgcctagccc |
| 19 | SaCr5 | gcctctttgtatccaacat |
| 20 | SaCr6 | gcacgctcctctgggacccctga |
| 21 | SaCr7 | gtgggggaggggacctgaa |
| 22 | SaCr8 | gaggggccatgaacggggg |

1.6.7 S. aureus Cas9-Based Repressor Gene Sequence

SEQ ID NO: 23 provides a nucleic acid sequence encoding HA-NLS-dSaCas9-NLS-KRAB. Residues 1-3 are a start codon. Residues 4-30 encode a HA tag. Residues 31-78 encode a first nuclear localization sequence (NLS). Residues 79-3234 encode S. aureus “dead” Cas9. Residues 103-105 encode the first inactivating mutation. Residues 1813-1815 encode the second inactivating mutation. Residues 3235-3282 encode a second NLS. Residues 3289-3597 encode KRAB. Residues 3598-3600 are a stop codon. All the residues are numbered based on SEQ ID NO: 23.

SEQ ID NO: 23
atgtacccatacgatgttccagattacgctGCCCCAAAGAAGAAGCGGAA
GGTCGGTATCCACGGAGTCCCAGCAGCCAAGCGGAACTACATCCTGGGCC
TGGCCATCGGCATCACCAGCGTGGGCTACGGCATCATCGACTACGAGACA
CGGGACGTGatcgATGCCGGCGTGCGGCTGTTCAAAGAGGCCAACGTGGA
AAACAACGAGGGCAGGCGGAGCAAGAGAGGCGCCAGAAGGCTGAAGCGGC
GGAGGCGGCATAGAATCCAGAGAGTGAAGAAGCTGCTGTTCGACTACAAC
CTGCTGACCGACCACAGCGAGCTGAGCGGCATCAACCCCTACGAGGCCAG
AGTGAAGGGCCTGAGCCAGAAGCTGAGCGAGGAAGAGTTCTCTGCCGCCC
TGCTGCACCTGGCCAAGAGAAGAGGCGTGCACAACGTGAACGAGGTGGAA
GAGGACACCGGCAACGAGCTGTCCACCAAAGAGCAGATCAGCCGGAACAG
CAAGGCCCTGGAAGAGAAATACGTGGCCGAACTGCAGCTGGAACGGCTGA
AGAAAGACGGCGAAGTGCGGGGCAGCATCAACAGATTCAAGACCAGCGAC
TACGTGAAAGAAGCCAAACAGCTGCTGAAGGTGCAGAAGGCCTACCACCA
GCTGGACCAGAGCTTCATCGACACCTACATCGACCTGCTGGAAACCCGGC
GGACCTACTATGAGGGACCTGGCGAGGGCAGCCCCTTCGGCTGGAAGGAC
ATCAAAGAATGGTACGAGATGCTGATGGGCCACTGCACCTACTTCCCCGA
GGAACTGCGGAGCGTGAAGTACGCCTACAACGCCGACCTGTACAACGCCC
TGAACGACCTGAACAATCTCGTGATCACCAGGGACGAGAACGAGAAGCTG
GAATATTACGAGAAGTTCCAGATCATCGAGAACGTGTTCAAGCAGAAGAA
GAAGCCCACCCTGAAGCAGATCGCCAAAGAAATCCTCGTGAACGAAGAGG
ATATTAAGGGCTACAGAGTGACCAGCACCGGCAAGCCCGAGTTCACCAAC
CTGAAGGTGTACCACGACATCAAGGACATTACCGCCCGGAAAGAGATTAT
TGAGAACGCCGAGCTGCTGGATCAGATTGCCAAGATCCTGACCATCTACC
AGAGCAGCGAGGACATCCAGGAAGAACTGACCAATCTGAACTCCGAGCTG
ACCCAGGAAGAGATCGAGCAGATCTCTAATCTGAAGGGCTATACCGGCAC
CCACAACCTGAGCCTGAAGGCCATCAACCTGATCCTGGACGAGCTGTGGC
ACACCAACGACAACCAGATCGCTATCTTCAACCGGCTGAAGCTGGTGCCC
AAGAAGGTGGACCTGTCCCAGCAGAAAGAGATCCCCACCACCCTGGTGGA
CGACTTCATCCTGAGCCCCGTCGTGAAGAGAAGCTTCATCCAGAGCATCA
AAGTGATCAACGCCATCATCAAGAAGTACGGCCTGCCCAACGACATCATT
ATCGAGCTGGCCCGCGAGAAGAACTCCAAGGACGCCCAGAAAATGATCAA
CGAGATGCAGAAGCGGAACCGGCAGACCAACGAGCGGATCGAGGAAATCA
TCCGGACCACCGGCAAAGAGAACGCCAAGTACCTGATCGAGAAGATCAAG
CTGCACGACATGCAGGAAGGCAAGTGCCTGTACAGCCTGGAAGCCATCCC
TCTGGAAGATCTGCTGAACAACCCCTTCAACTATGAGGTGGACCACATCA
TCCCCAGAAGCGTGTCCTTCGACAACAGCTTCAACAACAAGGTGCTCGTG
AAGCAGGAAGAAgcCAGCAAGAAGGGCAACCGGACCCCATTCCAGTACCT
GAGCAGCAGCGACAGCAAGATCAGCTACGAAACCTTCAAGAAGCACATCC
TGAATCTGGCCAAGGGCAAGGGCAGAATCAGCAAGACCAAGAAAGAGTAT
CTGCTGGAAGAACGGGACATCAACAGGTTCTCCGTGCAGAAAGACTTCAT
CAACCGGAACCTGGTGGATACCAGATACGCCACCAGAGGCCTGATGAACC
TGCTGCGGAGCTACTTCAGAGTGAACAACCTGGACGTGAAAGTGAAGTCC
ATCAATGGCGGCTTCACCAGCTTTCTGCGGCGGAAGTGGAAGTTTAAGAA
AGAGCGGAACAAGGGGTACAAGCACCAGCGCCAGGACGCCCTGATCATTG
CCAACGCCGATTTCATCTTCAAAGAGTGGAAGAAACTGGACAAGGCCAAA
AAAGTGATGGAAAACCAGATGTTCGAGGAAAAGCAGGCCGAGAGCATGCC
CGAGATCGAAACCGAGCAGGAGTACAAAGAGATCTTCATCACCCCCCACC
AGATCAAGCACATTAAGGACTTCAAGGACTACAAGTACAGCCACCGGGTG
GACAAGAAGCCTAATAGAGAGCTGATTAACGACACCCTGTACTCCACCCG
GAAGGACGACAAGGGCAACACCCTGATCGTGAACAATCTGAACGGCCTGT
ACGACAAGGACAATGACAAGCTGAAAAAGCTGATCAACAAGAGCCCCGAA
AAGCTGCTGATGTACCACCACGACCCCCAGACCTACCAGAAACTGAAGCT
GATTATGGAACAGTACGGCGACGAGAAGAATCCCCTGTACAAGTACTACG
AGGAAACCGGGAACTACCTGACCAAGTACTCCAAAAAGGACAACGGCCCC
GTGATCAAGAAGATTAAGTATTACGGCAACAAACTGAACGCCCATCTGGA
CATCACCGACGACTACCCCAACAGCAGAAACAAGGTCGTGAAGCTGTCCC
TGAAGCCCTACAGATTCGACGTGTACCTGGACAATGGCGTGTACAAGTTC
GTGACCGTGAAGAATCTGGATGTGATCAAAAAAGAAAACTACTACGAAGT
GAATAGCAAGTGCTATGAGGAAGCTAAGAAGCTGAAGAAGATCAGCAACC
AGGCCGAGTTTATCGCCTCCTTCTACAACAACGATCTGATCAAGATCAAC
GGCGAGCTGTATAGAGTGATCGGCGTGAACAACGACCTGCTGAACCGGAT
CGAAGTGAACATGATCGACATCACCTACCGCGAGTACCTGGAAAACATGA
ACGACAAGAGGCCCCCCAGGATCATTAAGACAATCGCCTCCAAGACCCAG
AGCATTAAGAAGTACAGCACAGACATTCTGGGCAACCTGTATGAAGTGAA
ATCTAAGAAGCACCCTCAGATCATCAAAAAGGGCAAAAGGCCGGCGGCCA
CGAAAAAGGCCGGCCAGGCAAAAAAGAAAAAGggatcCGATGCTAAGTCA
CTGACTGCCTGGTCCCGGACACTGGTGACCTTCAAGGATGTGTTTGTGGA
CTTCACCAGGGAGGAGTGGAAGCTGCTGGACACTGCTCAGCAGATCCTGT
ACAGAAATGTGATGCTGGAGAACTATAAGAACCTGGTTTCCTTGGGTTAT
CAGCTTACTAAGCCAGATGTGATCCTCCGGTTGGAGAAGGGAGAAGAGCC
CTGGCTGGTGGAGAGAGAAATTCACCAAGAGACCCATCCTGATTCAGAGA
CTGCATTTGAAATCAAATCATCAGTTCCGAAAAAGAAACGCAAAGtttaa

### 2. Additional Information

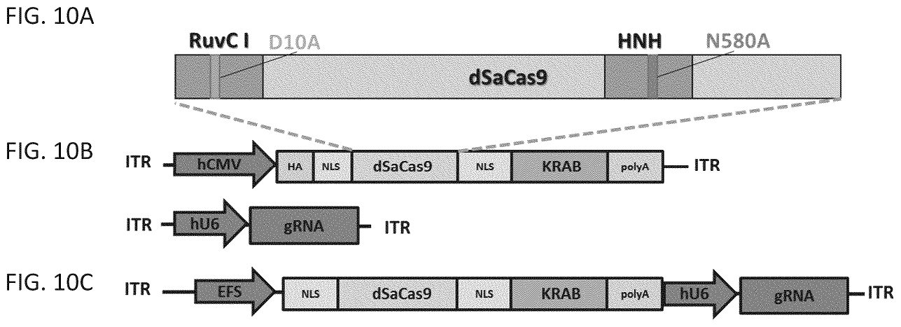

Engineered DNA-binding proteins that can be customized to target any gene in mammalian cells have enabled rapid advances in biomedical research and are a promising platform for gene therapies. The RNA-guided CRISPR/Cas9 system has emerged as a promising platform for programmable targeted gene regulation. Current Cas9 transcriptional repressors are based on Cas9 derived from the S. pyogenes bacterial strain. Fusion of catalytically inactive, “dead” Cas9 (dCas9) to the Kruppel-associated box (KRAB) domain generates a synthetic repressor capable of highly specific and potent silencing of target genes in cell culture experiments. However, a technology to deliver CRISPR/Cas9-based gene repressors in vivo has not been developed. Adeno-associated virus (AAV) vectors have been proposed for gene delivery of CRISPR/Cas9 components for in vivo studies and therapeutic applications. AAV vectors provide stable gene expression with low risk of mutagenic integration events, can be engineered to target tissues of interest in vivo, and are already in use in humans in clinical trials. However, gene delivery of S. pyogenes dCas9-KRAB in vivo is challenging because the size of the S. pyogenes dCas9 and KRAB domain fusion exceeds the packaging limit of standard AAV vectors. Recently, a smaller Cas9 nuclease protein derived from S. aureus was described for AAV delivery and in vivo gene editing. An S. aureus nuclease-null dCas9 was generated and fused to a synthetic KRAB repressor to create a programmable RNA-guided repressor for in vivo gene regulation (FIG. 10A). An AAV-based expression system was designed to deliver dCas9-KRAB fusion proteins and CRISPR gRNA targeting molecules in vivo (FIGS. 10B and 10C). When delivered intramuscularly using an AAV9 serotype vector, S. aureus dCas9-KRAB protein was expressed efficiently in skeletal muscle up to 8 weeks after delivery in a wild-type mouse model (FIG. 3D). Furthermore, it was demonstrated that S. aureus dCas9-KRAB is biologically active and can effectively silence an endogenous gene, acvr2b, in the injected muscle, heart and liver when delivered with a target guide RNA molecule (FIGS. 3E, 5B and 5D). This gene delivery system can be customized to target any endogenous gene by designing a new guide RNA molecule, enabling potent and stable gene repression in animal models and for human use.

### 3. Hypercholesterolemia

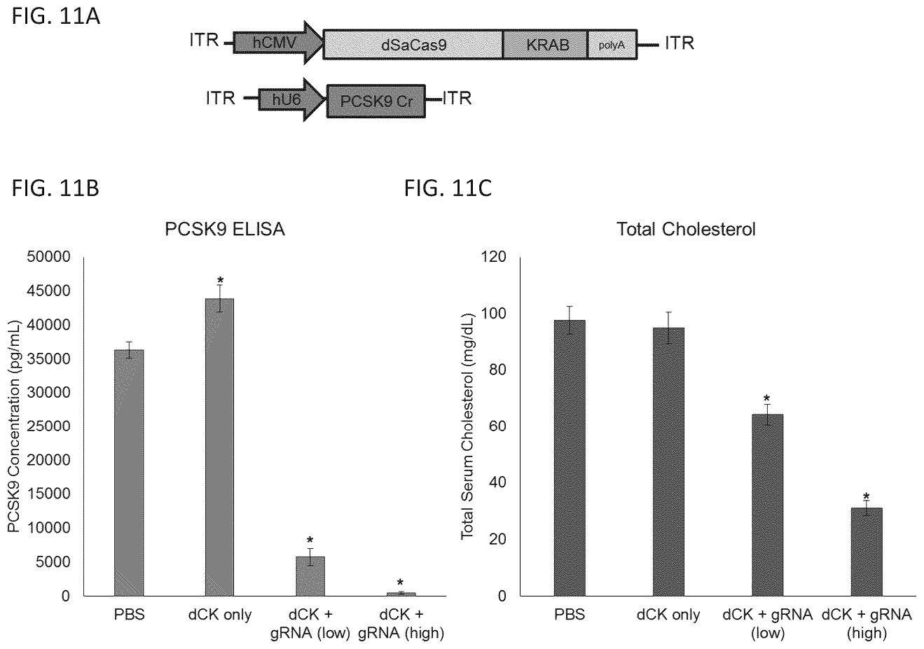

Hypercholesterolemia is a risk factor for cardiovascular disease, a leading cause of mortality in the United States. PCSK9 is a circulating protease that binds and facilitates degradation of low density lipoprotein (LDL) receptors. Individuals with naturally reduced PCSK9 demonstrate hypocholesterolemia, and silencing PCSK9 expression has been proposed as a mechanism to lower levels of harmful LDL cholesterol in the serum. RNA-guided CRISPR/Cas9-based transcriptional modulators can enable efficient and specific gene repression. An adeno-associated virus (AAV)-based gene modulation platform was developed using CRISPR/Cas9 repressors to enable targeted silencing of PCSK9 gene expression in vivo. To generate RNA-guided repressors, nuclease-inactive S. aureus Cas9 was fused to the KRAB domain, a motif found in mammalian transcription factors. CRISPR guide RNAs were targeted to the transcriptional start site region of the mouse PCSK9 gene. The dCas9-KRAB repressor and PCSK9 guide RNA (protospacer sequence: gagggaagggatacaggctgga (SEQ ID NO: 42); mm 10 coordinates: chr4 106464536-106464557) were expressed on separate adeno-associated viral vectors and delivered intravenously to wild-type mice (FIG. 11A). Two weeks after treatment, mice expressing dCas9-KRAB and PCSK9 guide RNA had significantly reduced circulating PCSK9 and total cholesterol levels in serum, compared to sham-treated and dCas9-KRAB only-treated controls (FIGS. 11B and 11C). The magnitude of PCSK9 repression and cholesterol reduction depended on the dose of AAV administered. Overall these results demonstrate that RNA-guided CRISPR/dCas9-KRAB repressors can effectively silence target liver gene expression in mouse models and show the potential of this technology for basic research and clinical applications.

### 4. Regulation of PCSK9 Expression In Vivo

### 4.1 Materials and Methods

### Plasmid Constructs and AAV Design

An inactive version of S. aureus Cas9 (dSaCas9) was created by introducing D10A and N580A mutations (Ran et al., Nature. 2015; 520:186-91, incorporated by reference herein in its entirety). A SaCas9 AAV expression plasmid (Addgene #61592) was received as a gift from the Zhang lab (Ran et al. Nature. 2005; 520:186-U98, incorporated by reference herein in its entirety). The nuclease-active SaCas9 was replaced with dSaCas9-KRAB. The C′ terminal 3×HA epitope tag was also removed and a single N′ terminal HA tag was incorporated for tracking protein expression. For the AAV-U6 gRNA plasmid, a U6-PCSK9 gRNA cassette was cloned into a pTR-eGFP backbone replacing the CMV with the gRNA.

### AAV Production

ITRs were verified by Smal digest before production. AAV-dSaCas9-KRAB and AAV-U6 PCSK9 gRNA were used to generate AAV9 in two separate batches by the Gene Transfer Vector Core at Schepens Eye Research Institute, Massachusetts Eye and Ear.

### Animal Studies

Animal studies were conducted with adherence to the guidelines for the care and use of laboratory animals of the National Institutes of Health (NIH). All the experiments with animals were approved by the Institutional Animal Care and Use Committee (IACUC) at Duke University. 6-8 week old C57Bl/6 mice (Jackson Labs) were anesthetized and maintained at 37° C. The tail vein was prepared and injected with 200 μL of AAV solution (2×10¹¹-4×10¹² viral genomes/total dose) or sterile PBS using a 31G needle. Low dose treatment was defined as 2×10¹¹ viral genomes per vector per mouse (vg/v/m), and moderate dose was defined as 4×10¹¹ vg/v/m. Mice were injected with a saline control, AAV-dSaCas9-KRAB alone, AAV-U6 PCSK9 gRNA alone, or a 1:1 mixture of AAV-dSaCas9-KRAB and AAV-U6 PCSK9 gRNA. Mice were fasted for 12-14 hours and submandibular vein blood collections were performed every two weeks, starting on day 0 four to six hours prior to tail vein injection. At 6 and 14 weeks post-injection, mice were euthanized by CO₂ inhalation, perfused with PBS, and tissue was collected into RNALater® (Life Technologies) for DNA and RNA, snap-frozen for protein analysis, or fixed in 4% PFA and embedded in OCT for histology.

qRT-PCR

Tissue samples were stored in RNALater (Ambion) and total RNA was isolated using the RNA Universal Plus Kit (Qiagen). cDNA synthesis was performed using the SuperScript VILO cDNA Synthesis Kit (Invitrogen). For genomic qPCR experiments, genomic DNA from tissue samples was isolated using a Blood and Tissue Kit (Qiagen). Quantitative real-time PCR (qRT-PCR) using QuantIT Perfecta Supermix was performed with the CFX96 Real-Time PCR Detection System (Bio-Rad) with the oligonucleotide primers optimized for 90-110% amplification efficiency. The results are expressed as fold-increase mRNA expression of the gene of interest normalized to Gapdh expression by the ΔΔCₜ method.

### RNA-Sequencing

mRNA was purified from total RNA using oligo (dT) Dynabeads (Invitrogen). First-strand cDNA was synthesized using the SuperScript VILO cDNA Synthesis Kit (Invitrogen) and second-strand cDNA was synthesized using DNA polymerase I (New England Biolabs). cDNA was purified using Agencourt AMPure XP beads (Beckman Coulter). Purified cDNA was treated with Nextera transposase (Illumina) for 5 min at 55° C. to simultaneously fragment and insert sequencing primers into the double-stranded cDNA. Transposase activity was halted using QG buffer (Qiagen) and fragmented cDNA was purified on AMPure XP beads. Indexed sequencing libraries were PCR-amplified and sequenced for 50-bp paired-end reads on an Illumina HiSeq 2000 instrument at the Duke Genome Sequencing Shared Resource. Reads aligned to the delivered AAV vector were removed from analysis. Filtered reads were then aligned to mouse RefSeq transcripts using Bowtie 2 (Langmead and Salzberg, Nat Methods. 2012; 9:357-9, incorporated by reference herein in its entirety). Statistical analysis, including multiple hypothesis testing, on three independent biological replicates was performed using DESeq (Anders and Huber, Genome Biol. 2010; 11: R106, incorporated by reference herein in its entirety).

### Western Blot

Minced tissue was lysed in RIPA buffer (Sigma), and the BCA assay (Pierce) was performed to quantify total protein. Lysates were mixed with LDS sample buffer (Invitrogen) and boiled for 5 min; equal amounts of total protein were run in NuPAGE Novex 4-12% Bis-Tris polyacrylamide gels (Life Technologies) and transferred to nitrocellulose membranes. Nonspecific antibody binding was blocked with 5% nonfat milk in TBS-T (50 mM Tris, 150 mM NaCl and 0.1% Tween-20) for 30 min. The membranes were then incubated with primary antibody in 5% milk in TBS-T: rabbit anti-LDLR diluted 1:1000 overnight at 4° C. or rabbit anti-GAPDH diluted 1:5000 for 60 min at room temperature. Membranes labeled with primary antibodies were incubated with anti-rabbit HRP-conjugated antibody (Sigma-Aldrich, A6154) diluted 1:5000 for 60 min and washed with TBS-T for 60 min. Membranes were visualized using the Immun-Star WesternC Chemiluminescence Kit (Bio-Rad) and images were captured using a ChemiDoc XRS+ system and processed using ImageLab software (Bio-Rad).

### Histology

A cross section of the median liver lobe was fixed overnight in 4% PFA and embedded in OCT using liquid nitrogen-cooled isopentane. 10 μm sections were cut onto pre-treated histological slides. Hematoxylin and eosin was used to reveal general liver histopathology.

### Serum Analysis

After harvest, serum was stored in one-time use aliquots at −80 C. Total cholesterol and LDL cholesterol levels were measured from serum via a colorimetric assay according to manufacturer's instructions (ThermoScientific Total Cholesterol Reagents #TR13421 and WakoChemical LDL Cholesterol #993-00404). PCSK9 serum protein levels were quantified by ELISA with a standard curve according to the manufacturer's instructions (R&D Systems #MPC900).

### 4.2 Results

Three independent studies were conducted, in which dSaCas9-KRAB repressor and PCSK9 guide RNA were delivered by AAV vectors to mice.

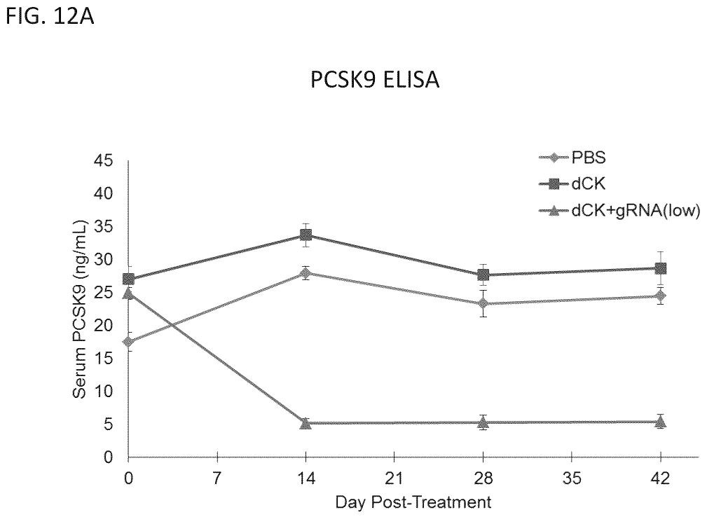

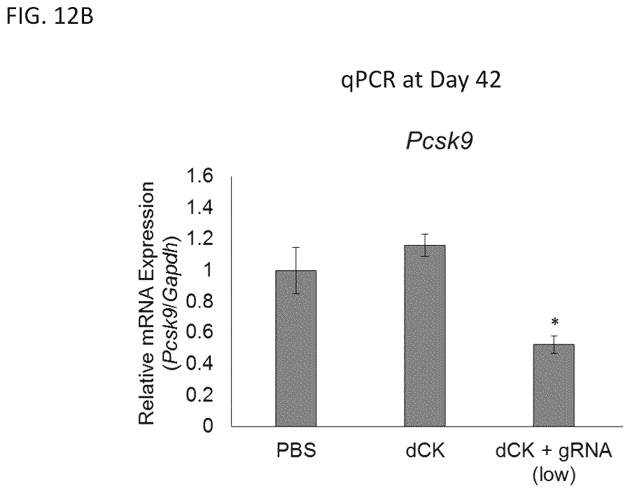

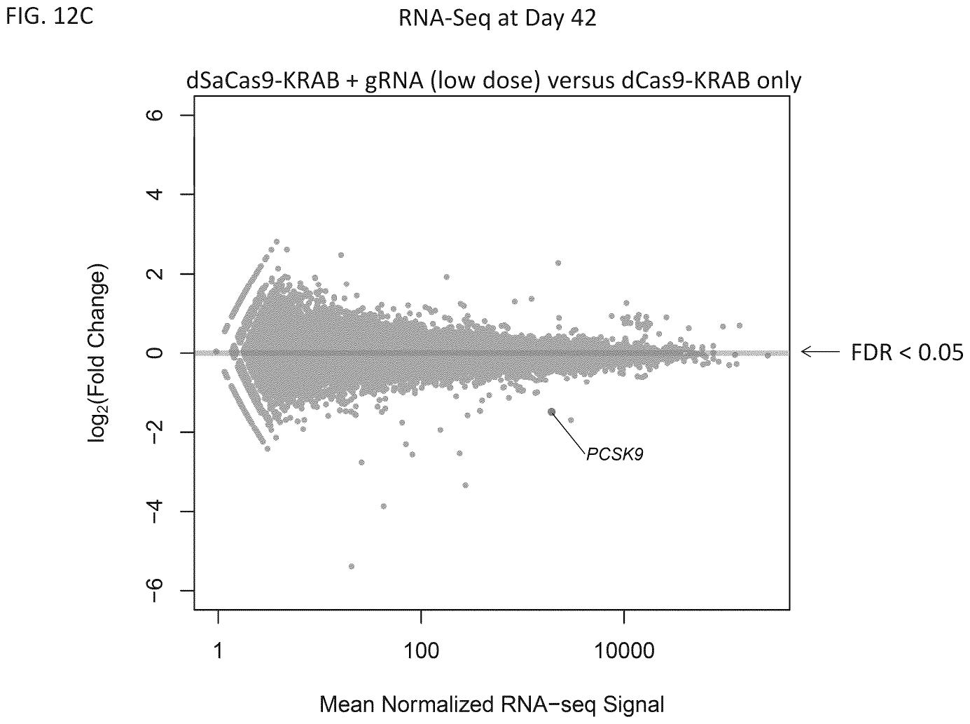

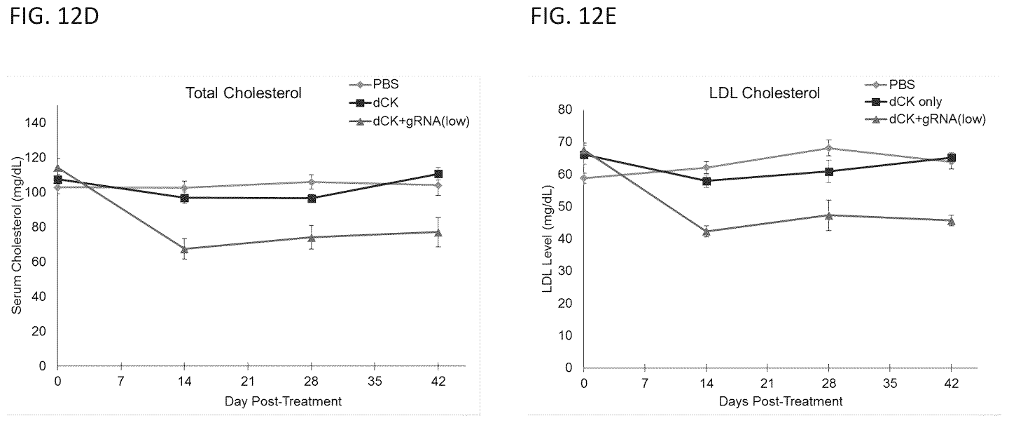

In the first study, mice were administered with PBS, AAV-dSaCas9-KRAB alone (1×10¹² total genomes/vector/mouse), or a low-dose 1:1 mixture of AAV-dSaCas9-KRAB and AAV-U6 PCSK9 gRNA (4×10¹¹ viral genomes/vector/mouse). Four mice were tested in each treatment group and followed for 6 weeks. As shown in FIG. 12A, low dose treatment with dSaCas9-KRAB and PCSK9 gRNA effectively lowered the serum levels of PCSK9 as measured by ELISA for at least 42 days post-treatment. Treatment with dSaCas9-KRAB alone did not reduce the serum levels of PCSK9 (FIG. 12A). Consistent with the reduction of PCSK9 protein levels, a reduction of PCSK9 mRNA levels in the liver was also observed in a qRT-PCR analysis (FIG. 12B) as well as a RNA-seq analysis (FIG. 12C). Total cholesterol and LDL cholesterol levels in the serum were measured using a colorimetric assay. As shown in FIGS. 12D and 12E, both the total and LDL cholesterol levels were reduced over the course of 42 days by the low-dose treatment with dSaCas9-KRAB and PCSK9 gRNA, compared to the PBS treatment or the treatment with dSaCas9-KRAB alone.

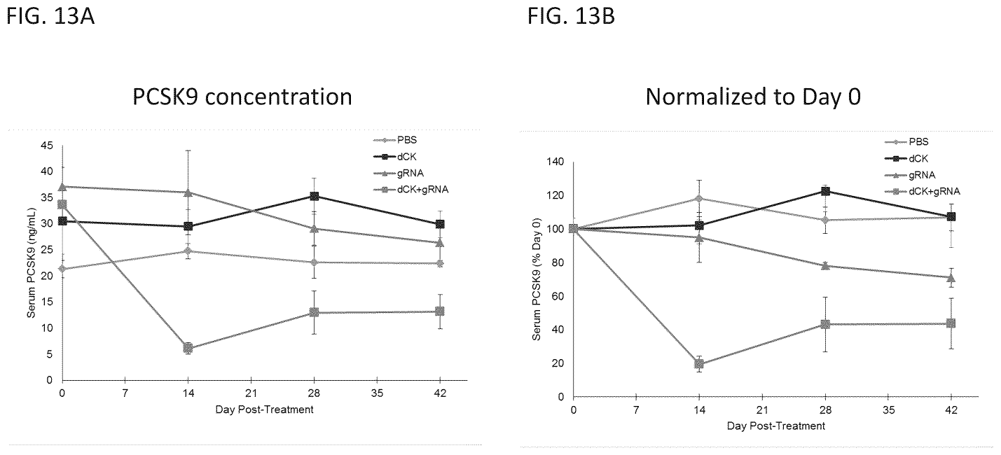

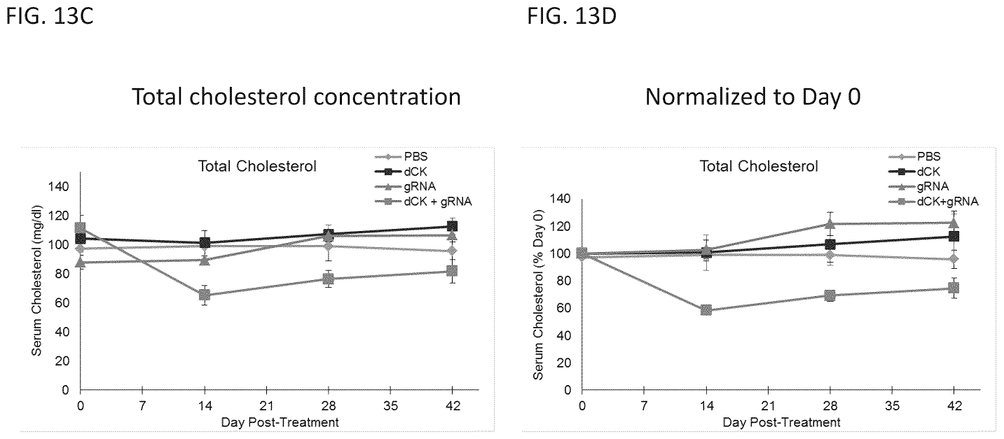

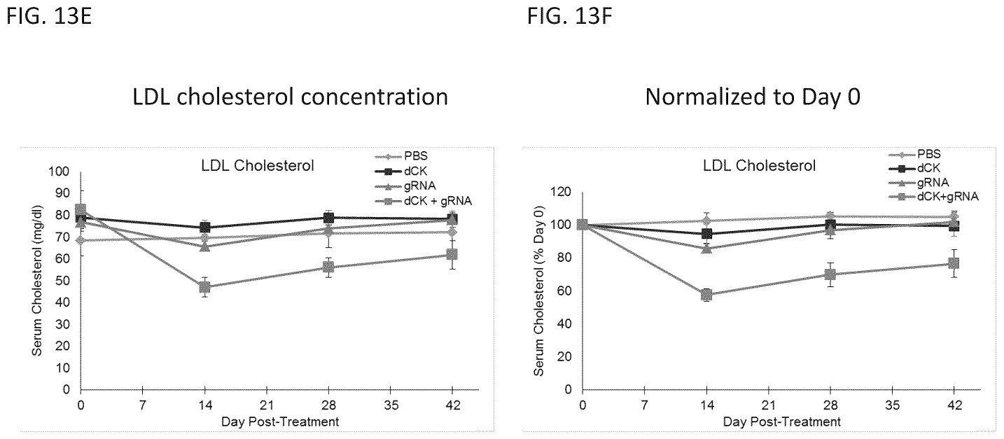

In the second study, mice were administered with PBS, AAV-dSaCas9-KRAB alone (4×10¹¹ total genomes/vector/mouse), AAV-U6 PCSK9 gRNA alone (4×10¹¹ total genomes/vector/mouse), or a moderate-dose 1:1 mixture of AAV-dSaCas9-KRAB and AAV-U6 PCSK9 gRNA (8×10¹¹ viral genomes/vector/mouse). Four mice were tested in each treatment group and followed for 6 weeks. Consistent with results from the low-dose study described above, treatment with a moderate dose of dSaCas9-KRAB and PCSK9 gRNA also reduced PCSK9 protein levels (FIGS. 13A and 13B), as well as total cholesterol levels (FIGS. 13C and 13D) and LDL cholesterol levels (FIGS. 13E and 13F) in the serum.

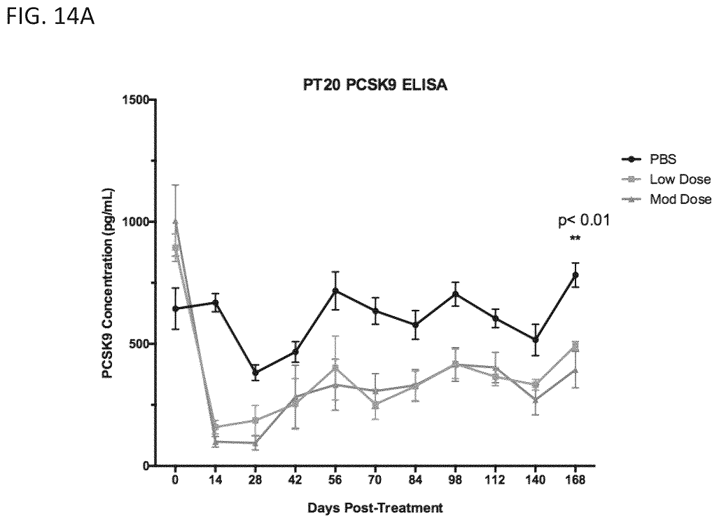

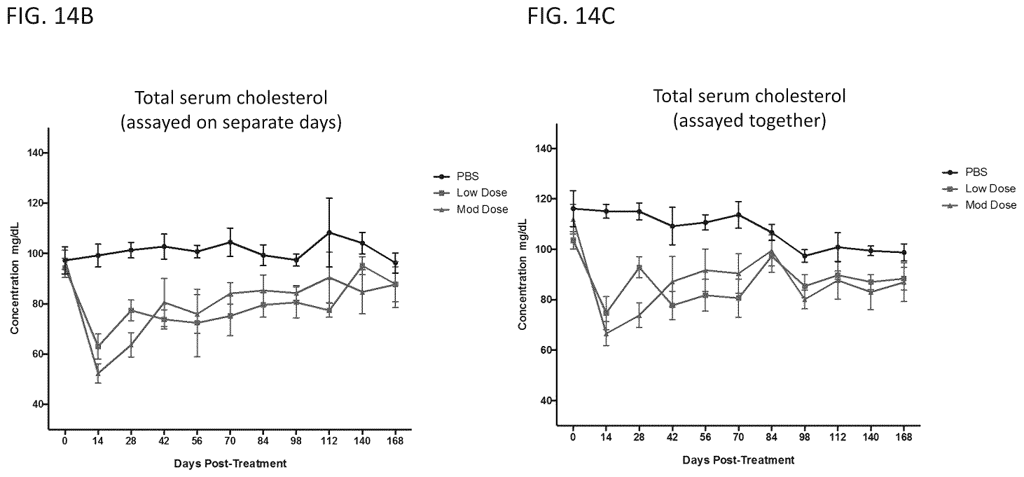

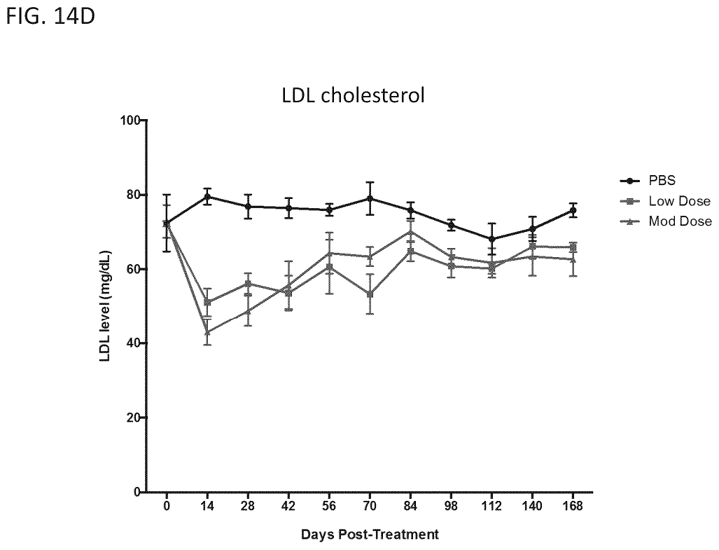

In the third study, mice were administered with PBS, a low-dose 1:1 mixture of AAV-dSaCas9-KRAB and AAV-U6 PCSK9 gRNA (4×10¹¹ viral genomes/vector/mouse), or a moderate-dose 1:1 mixture of AAV-dSaCas9-KRAB and AAV-U6 PCSK9 gRNA (8×10¹¹ viral genomes/vector/mouse). Four mice were tested in each group and followed for 24 weeks. As shown in FIG. 14A, both the low-dose and moderate-dose treatments with dSaCas9-KRAB and PCSK9 gRNA significantly lowered the serum PCSK9 levels for at least 168 days post-treatment. Both treatments also reduced total (FIGS. 14B and 14C) and LDL (FIG. 14D) cholesterol levels in the serum.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A method of modulating gene expression, in vivo, in a subject, the method comprising administering to the subject:
(a) a nucleic acid that encodes a fusion molecule comprising a dead Cas9 (dCas9) molecule fused to a modulator of gene expression,
wherein the dCas9 molecule comprises an amino acid sequence having at least 90% or greater identity to SEQ ID NO: 24 in which is present the amino acid mutations D10A and H840A, and
wherein the modulator of gene expression comprises a DNA methyltransferase; and
(b) a nucleic acid that encodes a gRNA that targets the fusion molecule to the promoter region of a PCSK9 gene,
in an amount sufficient to modulate expression of the PCSK9 gene.

2. The method of claim 1, wherein the DNA methyltransferase comprises DNMT3a-DNMT3L.

3. The method of claim 1, wherein the modulator of gene expression is fused to the C-terminus, N-terminus, or both, of the dCas9 molecule.

4. The method of claim 1, wherein the fusion molecule further comprises a nuclear localization sequence (NLS).

5. The method of claim 4, wherein the NLS comprises an amino acid sequence having at least 98% or greater identity to SEQ ID NO: 37 or 38.

6. The method of claim 4, wherein the NLS comprises the amino acid sequence of SEQ ID NO: 38.

7. The method of claim 1, wherein the dCas9 molecule comprises an amino acid sequence having at least 95% or greater identity to SEQ ID NO: 24 in which is present the amino acid mutations D10A and H840A.

8. The method of claim 1, wherein the dCas9 molecule comprises an amino acid sequence having at least 98% or greater identity to SEQ ID NO: 24 in which is present the amino acid mutations D10A and H840A.

9. The method of claim 1, wherein the nucleic acid of (a) and (b) are administered using a viral vector.

10. The method of claim 1, wherein the nucleic acid of (a) and (b) are administered using a non-viral delivery system.

11. The method of claim 7, wherein the fusion molecule further comprises a nuclear localization sequence (NLS).

12. The method of claim 7, wherein the modulator of gene expression is fused to the C-terminus, N-terminus, or both, of the dCas9 molecule.

13. The method of claim 11, wherein the NLS comprises an amino acid sequence having at least 98% or greater identity to SEQ ID NO: 37 or 38.

14. The method of claim 11, wherein the NLS comprises the amino acid sequence of SEQ ID NO: 38.

15. The method of claim 9, wherein the dCas9 molecule comprises an amino acid sequence having at least 95% or greater identity to SEQ ID NO: 24 in which is present the amino acid mutations D10A and H840A.

16. The method of claim 9, wherein the dCas9 molecule comprises an amino acid sequence having at least 98% or greater identity to SEQ ID NO: 24 in which is present the amino acid mutations D10A and H840A.

17. The method of claim 7, wherein the nucleic acid of (a) and (b) are administered using a viral vector.

18. The method of claim 7, wherein the nucleic acid of (a) and (b) are administered using a non-viral delivery system.

19. The method of claim 10, wherein the dCas9 molecule comprises an amino acid sequence having at least 95% or greater identity to SEQ ID NO: 24 in which is present the amino acid mutations D10A and H840A.

20. The method of claim 10, wherein the dCas9 molecule comprises an amino acid sequence having at least 98% or greater identity to SEQ ID NO: 24 in which is present the amino acid mutations D10A and H840A.
